SEQUENCE LISTING (631 sequences)

SEQ ID NO: 1 - DNA, 1293 bp, Arabidopsis thaliana
  CDS (1)..(1293)
atgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctga

SEQ ID NO: 2 - DNA, 1551 bp, Arabidopsis thaliana
  CDS (1)..(1551)
atggacggtgccggagaatcacgactcggtggtgatggtggtggtgatggttctgttggagttcagatccgacaaacacggatgctaccggattttctccagagcgtgaatctcaagtatgtgaaattaggttaccattacttaatctcaaatctcttgactctctgtttattccctctcgccgttgttatctccgtcgaagcctctcagatgaacccagatgatctcaaacagctctggatccatctacaatacaatctggttagtatcatcatctgttcagcgattctagtcttcgggttaacggtttatgttatgacccgacctagacccgtttacttggttgatttctcttgttatctcccacctgatcacctcaaagctccttacgctcggttcatggaacattctagactcaccggagatttcgatgactctgctctcgagtttcaacgcaagatccttgagcgttctggtttaggggaagacacttatgtccctgaagctatgcattatgttccaccgagaatttcaatggctgctgctagagaagaagctgaacaagtcatgtttggtgctttagataaccttttcgctaacactaatgtgaaaccaaaggatattggaatccttgttgtgaattgtagtctctttaatccaactccttcgttatctgcaatgattgtgaacaagtataagcttagaggtaacattagaagctacaatctaggcggtatgggttgcagcgcgggagttatcgctgtggatcttgctaaagacatgttgttggtacataggaacacttatgcggttgttgtttctactgagaacattactcagaattggtattttggtaacaagaaatcgatgttgataccgaactgcttgtttcgagttggtggctctgcggttttgctatcgaacaagtcgagggacaagagacggtctaagtacaggcttgtacatgtagtcaggactcaccgtggagcagatgataaagctttccgttgtgtttatcaagagcaggatgatacagggagaaccggggtttcgttgtcgaaagatctaatggcgattgcaggggaaactctcaaaaccaatatcactacattgggtcctcttgttctaccgataagtgagcagattcccttctttatgactctagttgtgaagaagctctttaacggtaaagtgaaaccgtatatcccggatttcaaacttgctttcgagcatttctgtatccatgctggtggaagagctgtgatcgatgagttagagaagaatctgcagctttcaccagttcatgtcgaggcttcgaggatgactcttcatcgatttggtaacacatcttcgagctccatttggtatgaattggcttacattgaagcgaagggaaggatgcgaagaggtaatcgtgtttggcaaatcgcgttcggaagtggatttaaatgtaatagcgcgatttgggaagcattaaggcatgtgaaaccttcgaacaacagtccttgggaagattgtattgacaagtatccggtaactttaagttattag

SEQ ID NO: 3 - DNA, 723 bp, Arabidopsis thaliana
  CDS (1)..(723)
atgtgttcattagagaaacgtgatcgtcttttcatactaaaactcaccggcgacggcgaacaccgtctaaacccaaccttattcgactctctccgctccaccatcaaccaaatccgatcagatccatcattttcacaatcagtactcatcacaacatcagatggtaaattcttctccaacggctacgatctcgctttagccgagtcaaatccttctctctctgttgtaatggacgcaaaacttagatccttagtcgccgatctaatctctcttcctatgccaacaatcgccgccgtcacaggtcacgcttccgccgcgggatgtattttagcgatgagtcatgattatgtattgatgcgtcgtgatagaggttttttgtatatgagtgaattggatattgagttgatagttccggcgtggttcatggctgttattaggggtaagattggttctccggcggccagaagggatgtgatgttgacggcggcgaaagtgacggcggatgtgggtgttaagatggggattgttgattcggcgtatggtagtgcggcggagacggttgaagccgccattaagttagatgaggagattgttcagagaggtggtgatggacacgtgtatggtaagatgagagagagtcttttaagagaggttcttattcatacgattggtgaatatgagagtggttcaagtgtggtgcgtagcactggatctaaactttag

SEQ ID NO: 4 - DNA, 2466 bp, Arabidopsis thaliana
  CDS (1)..(2466)
atggaaatgcccggtagaagatctaattacactttgcttagtcaattttctgacgatcaggtgtcagtttccgtcaccggagctcctccgcctcactatgattccttgtcgagcgaaaacaggagcaaccataacagcgggaacaccgggaaagctaaggcggagagaggcggatttgattgggatcctagcggtggtggtggtggtgatcataggttgaataatcaaccgaatcgggttgggaataatatgtatgcttcgtctctagggttgcaaaggcaatccagtgggagtagtttcggtgagagctctttgtctggggattattacatgcctacgctttctgcggcggctaacgagatcgaatctgttggatttcctcaagatgatgggtttaggcttggatttggtggtggtggaggagatttgaggatacagatggcggcggactccgctggagggtcttcatctgggaagagctgggcgcagcagacggaggagagttatcagctgcagcttgcattggcgttaaggctttcgtcggaggctacttgtgccgacgatccgaactttctggatcctgtaccggacgagtctgctttacggacttcgccaagttcagccgaaaccgtttcacatcgtttctgggttaatggctgcttatcgtactatgataaagttcctgatgggttttatatgatgaatggtctggatccctatatttggaccttatgcatcgacctgcatgaaagtggtcgcatcccttcaattgaatcattaagagctgttgattctggtgttgattcttcgcttgaagcgatcatagttgataggcgtagtgatccagccttcaaggaacttcacaatagagtccacgacatatcttgtagctgcattaccacaaaagaggttgttgatcagctggcaaagcttatctgcaatcgtatggggggtccagttatcatgggggaagatgagttggttcccatgtggaaggagtgcattgatggtctaaaagaaatctttaaagtggtggttcccataggtagcctctctgttggactctgcagacatcgagctttactcttcaaagtactggctgacataattgatttaccctgtcgaattgccaaaggatgtaaatattgtaatagagacgatgccgcttcgtgccttgtcaggtttgggcttgatagggagtacctggttgatttagtaggaaagccaggtcacttatgggagcctgattccttgctaaatggtccttcatctatctcaatttcttctcctctgcggtttccacgaccaaagccagttgaacccgcagtcgattttaggttactagccaaacaatatttctccgatagccagtctcttaatcttgttttcgatcctgcatcagatgatatgggattctcaatgtttcataggcaatatgataatccgggtggagagaatgacgcattggcagaaaatggtggtgggtctttgccacccagtgctaatatgcctccacagaacatgatgcgtgcgtcaaatcaaattgaagcagcacctatgaatgccccaccaatcagtcagccagttccaaacagggcaaatagggaacttggacttgatggtgatgatatggacatcccgtggtgtgatcttaatataaaagaaaagattggagcaggttcctttggcactgtccaccgtgctgagtggcatggctcggatgttgctgtgaaaattctcatggagcaagacttccatgctgagcgtgttaatgagttcttaagagaggttgcgataatgaaacgccttcgccaccctaacattgttctcttcatgggtgcggtcactcaacctccaaatttgtcaatagtgacagaatatttgtcaagaggtagtttatacagacttttgcataaaagtggagcaagggagcaattagatgagagacgtcgccttagtatggcttatgatgtggctaagggaatgaattatcttcacaatcgcaatcctccaattgtgcatagagatctaaaatctccaaacttattggttgacaaaaaatatacagtcaaggtttgtgattttggtctctcgcgattgaaggccagcacgtttctttcctcgaagtcagcagctggaacccccgagtggatggcaccagaagtcctgcgagatgagccgtctaatgaaaagtcagatgtgtacagcttcggggtcatcttgtgggagcttgctacattgcaacaaccatggggtaacttaaatccggctcaggttgtagctgcggttggtttcaagtgtaaacggctggagatcccgcgtaatctgaatcctcaggttgcagccataatcgagggttgttggaccaatgagccatggaagcgtccatcatttgcaactataatggacttgctaagaccattgatcaaatcagcggttcctccgcccaaccgctcggatttgtaa

SEQ ID NO: 5 - DNA, 1422 bp, Physcomitrella patens
  CDS (1)..(1422)
atggaaccccgcgtcggcaacaagtatcgccttggccggaaaattgggagtggttcctttggtgagatctacctggggaccaatctcgtgactcatgaggaggtcggcatcaagctggagagcatcaaggccaagcatccacaattgctttatgagtccaagttgtaccgtattcttcaaggaggaactgggattcccaacatcagatggtacggaattgaaggagactataatgtgatggttcttgatcttctgggacccagtcttgaagatcttttcaatttctgcagccggaaattctctttgaagacagttctcatgcttgccgaccagctgatcaatcgagtggagtatgtgcatgccaagagtttcctccacagggacataaagcctgacaatttcttgatggggctaggcaggcgagcaaatcaggtctatatgattgactttggtcttgcaaagaagtatcgcgatcccactactcatcagcacattccttatagagagaacaaaaatcttactggaaccgctcgatatgcaagtatcaacactcatcttggtattgaacaaagcaggagagatgatctggagtctcttggatatgttctcatgtatttcttgagaggcagcctgccttggcaaggaatgaaagcaggaaccaagaagcagaagtatgaaaaaatcagtgagaaaaagatgtccacccctatagagttcctttgtaaagcttacccgtctgagtttgcttcatacttccactactgtcggtctcttcggttcgatgacaaaccggactatgcttacctgaagagaattttccgagatctcttcattcgtgagggttttcagtttgattatgttttcgactggacgattttgaagtatcagcaaacacatttttctggtggtcctctccgtccagcggctgcggcgggaggttcaagtggagcagcagcagcagcggcagcaggaattggtacagtcccaagagacgcccagcgagcaattgagcctactgatgttgccgctcgaactcgaatggttggtgcgactcgctctagtggattaaatccactggacgcgtcaaagcataagagtactagcccagatgaagccgcttctaaggacatagcccttagcggtcttgcagaaccagagcgcacgcatgcttcttcgtttgtgcgggggagctcatcatcaaggagagctgttgttggatgtgctaggccagcagggtcaacagaggcgggagatggaacgcgggtgttggctggcaaaatgggccccactagcctgcgcacatcagcaggaatgcagaggagctctccggtggcatctacggatcccaagcggacgggacgagattcttatgctggaaactccggaagaaatcctagttcctctcgaaattcgaaagagtga

SEQ ID NO: 6 - DNA, 2427 bp, Arabidopsis thaliana
  CDS (1)..(2427)
atggtaaaggaaactctaattcctccgtcatctacgtcaatgacgaccggaacatcttcttcttcgtctctttcaatgacgttatcctcaacaaacgcgttatcgtttttgtcgaaaggatggagagaggtatgggattcagcagatgcggatttgcagctgatgcgagacagagctaactctgttaagaatctagcatcaacgttcgatagagagatcgagaatttcctcaataactcggcgaggtctgcgtttcccgttggttcaccatcggcgtcgtctttctcaaatgaaattggtatcatgaagaagcttcagccgaagatttcggagtttcgtagggtttattcggcgccggagattagtcgcaaggttatggagagatggggacctgcgagagcgaagcttggaatggatctatcggcgattaagaaggcgattgtgtctgagatggaattggatgagcgtcagggagttttggagatgagtagattgaggagacggcgtaatagtgatagggttaggtttacggagtttttcgcggaggctgagagagatggagaagcttatttcggtgattgggaaccgattaggtctttgaagagtagatttaaagagtttgagaaacgaagctcgttagaaatattgagtggattcaagaacagtgaatttgttgagaagctcaaaaccagctttaaatcaatttacaaagaaactgatgaggctaaggatgtccctccgttggatgtacctgaactgttggcatgtttggttagacaatctgaaccttttcttgatcagattggtgttagaaaggatacatgtgaccgaatagtagaaagcctttgcaaatgcaagagccaacaactttggcgtctgccatctgcacaagcatccgatttaattgaaaatgataaccatggagttgatttggatatgaggatagccagtgttcttcaaagcacaggacaccattatgatggtgggttttggactgattttgtgaagcctgagacaccggaaaacaaaaggcatgtggcaattgttacaacagctagtcttccttggatgaccggaacagctgtaaatccgctattcagagcggcgtatttggcaaaagctgcaaaacagagtgttactctcgtggttccttggctctgcgaatctgatcaagaactagtgtatccaaacaatctcaccttcagctcacctgaagaacaagagagttatatacgtaaatggttggaggaaaggattggtttcaaggctgattttaaaatctccttttacccaggaaagttttcaaaagaaaggcgcagcatatttcctgctggtgacacttctcaatttatatcgtcaaaagatgctgacattgctatacttgaagaacctgaacatctcaactggtattatcacggcaagcgttggactgataaattcaaccatgttgttggaattgtccacacaaactacttagagtacatcaagagggagaagaatggagctcttcaagcattttttgtgaaccatgtaaacaattgggtcacacgagcgtattgtgacaaggttcttcgcctctctgcggcaacacaagatttaccaaagtctgttgtatgcaatgtccatggtgtcaatcccaagttccttatgattggggagaaaattgctgaagagagatcccgtggtgaacaagctttctcaaaaggtgcatacttcttaggaaaaatggtgtgggctaaaggatacagagaactaatagatctgatggctaaacacaaaagcgaacttgggagcttcaatctagatgtatatgggaacggtgaagatgcagtcgaggtccaacgtgcagcaaagaaacatgacttgaatctcaatttcctcaaaggaagggaccacgctgacgatgctcttcacaagtacaaagtgttcataaaccccagcatcagcgatgttctatgcacagcaaccgcagaagcactagccatggggaagtttgtggtgtgtgcagatcacccttcaaacgaattctttagatcattcccgaactgcttaacttacaaaacatccgaagactttgtgtccaaagtgcaagaagcaatgacgaaagagccactacctctcactcctgaacaaatgtacaatctctcttgggaagcagcaacacagaggttcatggagtattcagatctcgataagatcttaaacaatggagagggaggaaggaagatgcgaaaatcaagatcggttccgagctttaacgaggtggtcgatggaggattggcattctcacactatgttctaacagggaacgatttcttgagactatgcactggagcaacaccaagaacaaaagactatgataatcaacattgcaaggatctgaatctcgtaccacctcacgttcacaagccaatcttcggctggtag

SEQ ID NO: 7 - DNA, 2394 bp, Arabidopsis thaliana
  CDS (1)..(2394)
atgtgtgttgtgattggtctcaagtcatgggtaatggttttggttgttatctttattagatatgtagcccagggaaaggggatattgcagtcccaccagctgattgatgagttccttaagactgtgaaagttgatggaacattagaagatcttaacaaaagtccattcatgaaagttctgcagtctgcagaggaagccatagttttgcctccatttgttgctttggctatacgtcccagacctggtgttagggaatatgtccgtgtgaatgtgtatgagctgagcgtagatcatttaactgtttctgaatatcttcggtttaaggaagagctcgttaatggccatgccaatggagattatctccttgaacttgattttgaacctttcaatgcaacattgcctcgcccaactcgttcatcatccattgggaatggggttcagttcctcaatcgtcacctctcttcaattatgttccgtaacaaagaaagcatggagcctttgcttgagtttctccgcactcacaaacatgatggccgtcctatgatgctgaatgatcgaatacagaatatccccatacttcagggagctttggcaagagcagaggagttcctttctaaacttcctctggcaacaccatactctgaattcgaatttgaactacaagggatgggatttgaaaggggatggggtgacacagcacagaaggtttcagaaatggtgcatcttcttctggacatactccaggcacctgatccttctgtcttggagacgtttctaggaaggattcctatggtgttcaatgttgtgattttgtctccgcatggttactttggccaagccaatgtcttgggtctgcctgatactggtggacaggttgtctacattcttgatcaagtacgtgcattggaaaatgagatgctccttaggatacagaagcaaggactggaagttattccaaagattctcattgtaacaagactgctacccgaagcaaagggaacaacgtgcaaccagaggttagaaagagttagtggtacagaacacgcacacattctgcgaataccatttaggactgaaaagggaattcttcgcaagtggatctcaaggtttgatgtctggccatacctggagacttttgcagaggatgcatcaaatgaaatttctgcggagttgcagggtgtaccaaatctcatcattggcaactacagtgatggaaatctcgttgcttctttgttagctagtaagctaggtgtgatacagtgtaatattgctcatgctttagagaaaaccaagtaccccgagtctgacatttactggagaaaccatgaagataagtatcacttttcaagtcagttcactgcagatctaattgccatgaataatgccgatttcatcatcaccagcacataccaagagattgcgggaagcaagaacaatgttgggcaatacgagagccacacagctttcactatgcctggtctttaccgagttgttcatggaattgatgtctttgatcctaagtttaatatggtctctccaggagctgatatgaccatatactttccatattccgacaaggaaagaagactcactgcccttcatgagtcaattgaagaactcctctttagtgccgaacagaatgatgagcatgttggtttactgagcgaccaatcgaagccaatcatcttctctatggcaagacttgacagggtgaaaaacttgactgggctagttgaatgctatgccaagaatagcaagcttagagagcttgcaaatcttgttatagtcggtggctacatcgatgagaatcagtccagggatagagaggaaatggctgagatacaaaagatgcacagcctgattgagcagtatgatttacacggtgagtttaggtggatagctgctcaaatgaaccgtgctcgaaatggtgagctttaccgttatatcgcagacacaaaaggtgtttttgttcagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggtacacatggaagaagtactcagagagactgcttaccctggctggagtctatgcattctggaaacatgtgtctaagctcgaaaggagagaaacacgacgttacctagagatgttttactcattgaaatttcgtgatttggccaattcaatcccgctggcaacagatgagaactga

SEQ ID NO: 8 - DNA, 1179 bp, Arabidopsis thaliana
  CDS (1)..(1176)
atggcgacttttgctgaacttgttttatcgacttctcgctgtacatgcccttgccgttcattcactagaaaacccctaattcgtccccctttatctggtctgcgtctccccggtgataccaaaccattgtttcgttccggacttggtcggatttctgttagccggcgtttcctcacggccgttgctcgagctgaatcagaccagcttggtgatgatgaccactcaaagggaattgatagaatccataacttgcagaatgtggaagataagcagaagaaagcaagccagcttaagaaaagagtgatctttggtattggcattggtttacctgttggatgtgttgtgttagctggaggatgggttttcactgtagctttagcatcttctgtttttatcggttcccgcgaatatttcgagcttgttagaagtagaggcatagctaaaggaatgactcctcctccacgatatgtatctcgagtttgctcggttatatgtgcccttatgcccatacttacactgtactttggtaacattgatatattggtgacatctgcagcatttgttgttgcaatagcattgttagtacaaagaggatccccacgttttgctcagctgagtagtacaatgtttggtctgttttactgtggttatctcccttctttctgggttaagcttcgctgtggtttagctgctcctgcgcttaacactggtatcggaaggacatggccaattcttcttggtggtcaagctcattggacagttggacttgtggcaacattgatttctttcagcggtgtaattgcgacagacacatttgcttttctcggtggaaagacttttggtaggacacctcttactagtattagtcccaagaagacatgggaaggaactattgtaggacttgttggttgtatagccattaccatattactctctaaatatctcagttggccacaatctctgttcagctcagtagcttttgggtttcttaacttctttgggtcagtctttggtgatcttactgaatcaatgatcaagcgtgatgctggcgtcaaagactctggttcacttatcccaggacacggtggaatattagatagagttgatagttacattttcaccggcgcattagcttattcattcatcaaaacatccctaaaactttacggagtttgatga

SEQ ID NO: 9 - DNA, 831 bp, Pisum sativum
cgcaattttttgtgaagctgagggaggattggattttacacctattcaaaagtcattcaaagtttgtccctccattcaaggatgaatgtagatttttcaagcatcaaacacaagaatcactagcataacatgctttgaaacccacacacttaaattaatgttaggaatatcaaatccaatataaaatcatagttgtcaattacatactcaatcaagtccctttcttttacccaataaacatcaacatattgcttcttccattaagcatataaacatcaaagtctaaaactagcaaaatgttgtttttaggatgacacatttcatacatagtttaaaagatacttgattcgattacaaaaagaaattaccaatagtttagcacaaagtctaaagcataattaaagcatcacatgtgcagatttatgaaaaaaagattaagattgcccctttcatcacgggtcgaataatagcactacttgtcactacatgttaaaaaaatgtcctctagtacatcaaactttttccattgattccccttatccatgaaaaaaataaacaaattcttaagacacaaaaaaatggccccacatccttttttctggcctagtttgtttgaattcattctaactcttgaatatgtaacgaggcccactaaaaatcaatcaatgatttaacataaaaaatgaatagtttaattccaatttgctgcaacatggtccgtgaatatgactcacgagaaagatatatcaaaatatcaaaatttcatagtttttttcaccatataaacctcatcactcattctatttttttaagtgcaaagcttcatagtta

SEQ ID NO: 10 - DNA, 674 bp, Unknown
  USP Promoter
caaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgatgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagc

SEQ ID NO: 11 - DNA, 764 bp, Unknown
  LeB4 Promoter
gagttaccatttctttttcctgcatctcaatagtatatagggtatcaaatagtgattatccaaacttaaataagttagaggaaacaccaagatatgccatatactctcatatttgacactatgattcaaagttgcacttgcataaaacttattaattcaatagtaaaaccaaacttgtgcgtgatacagttaaaatgactaaactactaattaaggtccctcccattagtaaataagttattttcttagaaaaagaaaataataaaaagaatgacgagtctatctaaatcatattaacaagtaatacatattgattcattcgatggaggaggccaataattgtagtaaacaagcagtgccgaggttaatatatgctcaagacagtaaataatctaaatgaattaagacagtgatttgcaaagagtagatgcagagaagagaactaaagatttgctgctacacgtatataagaatagcaacagatattcattctgtctctttgtggaatatggatatctactaatcatcatctatctgtgaagaataaaagaagcggccacaagcgcagcgtcgcacatatgatgtgtatcaaattaggactccatagccatgcatgctgaagaatgtcacacacgttctgtcacacgtgttactctctcactgttctcctcttcctataaatcaccgcgccacagcttctccacttcaccacttcaccacttcactcacaatccttcattagttgtttactatcacagtcacag

SEQ ID NO: 12 - DNA, 2769 bp, Unknown
  LEB4 Promoter
gtggaattcgagggggatctgtcgtctcaaactcattcatcagaaccttcttgaacttagttatctcttgttcagagcttcctgttagcaatatgtcatcaacatataaacatgtcccagaagccagaagatagaagttggatgatagaagtaaagtaatgttactggtggagtaccacaatacaagttcatacaaactttattgtccagaaactaacaaagttgagttcagcatagatgaaagacaaaaagaatatattaaatgacggctgcaaaataaggagtaatgaatacattgacctacctactactaggctatttatacacaatattagggtataataaaatattaaaataccctctatcagacttagtcaataagacattcctaaaatataaattatttccaacaataatttgtctcaaataaaatatagaggtgcaaaagttaaactaagagtgcaaagtaaaattttgagagggctcaaaattgaatataataacaatattagtgtagtttaagaaaactcaggggatgcagttgaactccctcaactgtacgtagctcctcccctggatgcagtgtaaagatttgaagatatattttagtactttggatattgtaggccagagggtgttgaagataaaggttcaggaactaacacattcatccacaacttctatgtgtccatcgtcagtgaaatacatgccaaataggggagttaagaagagtagaaagggtcaagatagtgatgtgcatcgtgatccttcataatgggagtgtggtgagggctcgcatgggagtcatactacaaagagatcatgcataaaaccaactagaagtcaactgtcaagtatgacggctgacaattaaccgtccaccaaatcttccagacatgtttacttgtcccagttttctgatttcttatatccatacattgatgacattattgatgttggtggcgatggagattggggttttcatgctattacagctttacttggatggggtgaagagtcatagcctttgattcagacgcagttagatactcaagttcatcaacaccctcaattgttttttaagttgttttgtgacacgatctctacagttagaaatgcgttacgagtagaacacttggctgtgcagggtatagataaatgaatgacgatttatgatatgggttaccctattgcttctagatacaatgtcgtatttgtctcccttccaaaagacttaacatcacgttttttcctcttgccttatctccacctatgtatacaagcaggcataaaatcattgttgttggttttgtcaacaacaatcattgagtttaggtaaagttgaaacttgattgtccattacctcttgtcactgactgttgaagacagaattgtactgactgtatatatcaacatatgcgagacgcgttaggcagtggaaagacgtagttaggatgtcatcataatttgtttcgtatttttatatgtagcacagtttttatatgtatatattttatcgggtagttttttatcgattcagttatttgagaaaaagtaatgcagacaaaaagtggaaaagacaatctgactgtacataagaaatttccaatttttgaaatttttttataattatcagaaattttaaaatttccgataaaaacatacatgtatagatcgaaaatttcaaatttctagtactttcaaatttcttgcagtaaaagttgtaattttttaaaaatttacgataatttacagtatttaaaaaaaaatccaatcttaaataaagggtataagaataaaagcactcatgtggagtggcaggtttcgtcacaccctaagaacatccctaaatacaccacatatgtataagtattaagtgattgatgttaagtgaaacgaaaatatttatatgtgaaatttaatattcagcttacttgattaaactccatagtgacccaataagtgctaacttttactgtctttacctttaaatgttatattgatttatttatgcatttctttttcctgcatctcaatagtatatagggtatcaaatagtgattatccaaacttaaataagttagaggaaacaccaagatatgccatatactctcaaatttgacactatgattcaaagttgcacttgcataaaacttattaattcaatagtaaaaccaaacttgtgcgtgatacagttaaaatgactaaactactaattaaggtccctcccattagtaaataagttatttttttagaaaaagaaaataataaaaagaatgacgagtctatctaaatcatattaacaagtaatacatattgattcattcgatggaggaggccaataattgtagtaaacaagcagtgccgaggttaatatatgctcaagacagtaaataatctaaatgaattaagacagtgatttgcaaagagtagatgcagagaagagaactaaagatttgctgctacacgtatataagaatagcaacagatattcattctgtctctttgtggaatatggatatctactaatcatcatctatctgtgaagaataaaagaagcggccacaagcgcagcgtcgcacatatgatgtgtatcaaattaggactccatagccatgcatgctgaagaatgtcacacacgttctgtcacacgtgttactctctcactgttctcctcttcctataaatcaccgcgccacagcttctccacttcaccacttcaccacttcactcacaatccttcattagttgtttactatcacagtcaca

SEQ ID NO: 13 - DNA, 1039 bp, Unknown
  Conlinin Promoter
ttagcagatatttggtgtctaaatgtttattttgtgatatgttcatgtttgaaatggtggtttcgaaaccagggacaacgttgggatctgatagggtgtcaaagagtattatggattgggacaatttcggtcatgagttgcaaattcaagtatatcgttcgattatgaaaattttcgaagaatatcccatttgagagagtctttacctcattaatgtttttagattatgaaattttatcatagttcatcgtagtctttttggtgtaaaggctgtaaaaagaaattgttcacttttgttttcgtttatgtgaaggctgtaaaagattgtaaaagactattttggtgttttggataaaatgatagtttttatagattcttttgcttttagaagaaatacatttgaaattttttccatgttgagtataaaataccgaaatcgattgaagatcatagaaatattttaactgaaaacaaatttataactgattcaattctctccatttttatacctatttaaccgtaatcgattctaatagatgatcgattttttatataatcctaattaaccaacggcatgtattggataattaaccgatcaactctcacccctaatagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacacct

SEQ ID NO: 14 - DNA, 670 bp, Unknown
  E9 Terminator
ggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgaccgggtacc

SEQ ID NO: 15 - DNA, 216 bp, Unknown
  A7 Terminator
ctgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcga

SEQ ID NO: 16 - DNA, 194 bp, Unknown
  OCS Terminator
ctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtcc

SEQ ID NO: 17 - DNA, 297 bp, Unknown
  LeBT Terminator
atcctgcaatagaatgttgaggtgaccactttctgtaataaaataattataaaataaatttagaattgctgtagtcaagaacatcagttctaaaatattaataaagttatggccttttgacatatgtgtttcgataaaaaaatcaaaataaattgagatttattcgaaatacaatgaaagtttgcagatatgagatatgtttctacaaaataataacttaaaactcaactatatgctaatgtttttcttggtgtgtttcatagaaaattgtatccgtttcttagaaaatgctcgtaa

SEQ ID NO: 18 - protein, 430 aa, Arabidopsis thaliana
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLV

SEQ ID NO: 19 - protein, 516 aa, Arabidopsis thaliana
MDGAGESRLGGDGGGDGSVGVQIRQTRMLPDFLQSVNLKYVKLGYHYLISNLLTLCLFPLAVVISVEASQMNPDDLKQLWIHLQYNLVSIIICSAILVFGLTVYVMTRPRPVYLVDFSCYLPPDHLKAPYARFMEHSRLTGDFDDSALEFQRKILERSGLGEDTYVPEAMHYVPPRISMAAAREEAEQVMFGALDNLFANTNVKPKDIGILVVNCSLFNPTPSLSAMIVNKYKLRGNIRSYNLGGMGCSAGVIAVDLAKDMLLVHRNTYAVVVSTENITQNWYFGNKKSMLIPNCLFRVGGSAVLLSNKSRDKRRSKYRLVHVVRTHRGADDKAFRCVYQEQDDTGRTGVSLSKDLMAIAGETLKTNITTLGPLVLPISEQIPFFMTLVVKKLFNGKVKPYIPDFKLAFEHFCIHAGGRAVIDELEKNLQLSPVHVEASRMTLHRFGNTSSSSIWYELAYIEAKGRMRRGNRVWQIAFGSGFKCNSAIWEALRHVKPSNNSPWEDCIDKYPVTLSY

SEQ ID NO: 20 - protein, 240 aa, Arabidopsis thaliana
MCSLEKRDRLFILKLTGDGEHRLNPTLFDSLRSTINQIRSDPSFSQSVLITTSDGKFFSNGYDLALAESNPSLSVVMDAKLRSLVADLISLPMPTIAAVTGHASAAGCILAMSHDYVLMRRDRGFLYMSELDIELIVPAWFMAVIRGKIGSPAARRDVMLTAAKVTADVGVKMGIVDSAYGSAAETVEAAIKLDEEIVQRGGDGHVYGKMRESLLREVLIHTIGEYESGSSVVRSTGSKL

SEQ ID NO: 21 - protein, 821 aa, Arabidopsis thaliana
MEMPGRRSNYTLLSQFSDDQVSVSVTGAPPPHYDSLSSENRSNHNSGNTGKAKAERGGFDWDPSGGGGGDHRLNNQPNRVGNNMYASSLGLQRQSSGSSFGESSLSGDYYMPTLSAAANEIESVGFPQDDGFRLGFGGGGGDLRIQMAADSAGGSSSGKSWAQQTEESYQLQLALALRLSSEATCADDPNFLDPVPDESALRTSPSSAETVSHRFWVNGCLSYYDKVPDGFYMMNGLDPYIWTLCIDLHESGRIPSIESLRAVDSGVDSSLEAIIVDRRSDPAFKELHNRVHDISCSCITTKEVVDQLAKLICNRMGGPVIMGEDELVPMWKECIDGLKEIFKVVVPIGSLSVGLCRHRALLFKVLADIIDLPCRIAKGCKYCNRDDAASCLVRFGLDREYLVDLVGKPGHLWEPDSLLNGPSSISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 22 - protein, 473 aa, Physcomitrella patens
MEPRVGNKYRLGRKIGSGSFGEIYLGTNLVTHEEVGIKLESIKAKHPQLLYESKLYRILQGGTGIPNIRWYGIEGDYNVMVLDLLGPSLEDLFNFCSRKFSLKTVLMLADQLINRVEYVHAKSFLHRDIKPDNFLMGLGRRANQVYMIDFGLAKKYRDPTTHQHIPYRENKNLTGTARYASINTHLGIEQSRRDDLESLGYVLMYFLRGSLPWQGMKAGTKKQKYEKISEKKMSTPIEFLCKAYPSEFASYFHYCRSLRFDDKPDYAYLKRIFRDLFIREGFQFDYVFDWTILKYQQTHFSGGPLRPAAAAGGSSGAAAAAAAGIGTVPRDAQRAIEPTDVAARTRMVGATRSSGLNPLDASKHKSTSPDEAASKDIALSGLAEPERTHASSFVRGSSSSRRAVVGCARPAGSTEAGDGTRVLAGKMGPTSLRTSAGMQRSSPVASTDPKRTGRDSYAGNSGRNPSSSRNSKE

SEQ ID NO: 23 - protein, 808 aa, Arabidopsis thaliana
MVKETLIPPSSTSMTTGTSSSSSLSMTLSSTNALSFLSKGWREVWDSADADLQLMRDRANSVKNLASTFDREIENFLNNSARSAFPVGSPSASSFSNEIGIMKKLQPKISEFRRVYSAPEISRKVMERWGPARAKLGMDLSAIKKAIVSEMELDERQGVLEMSRLRRRRNSDRVRFTEFFAEAERDGEAYFGDWEPIRSLKSRFKEFEKRSSLEILSGFKNSEFVEKLKTSFKSIYKETDEAKDVPPLDVPELLACLVRQSEPFLDQIGVRKDTCDRIVESLCKCKSQQLWRLPSAQASDLIENDNHGVDLDMRIASVLQSTGHHYDGGFWTDFVKPETPENKRHVAIVTTASLPWMTGTAVNPLFRAAYLAKAAKQSVTLVVPWLCESDQELVYPNNLTFSSPEEQESYIRKWLEERIGFKADFKISFYPGKFSKERRSIFPAGDTSQFISSKDADIAILEEPEHLNWYYHGKRWTDKFNHVVGIVHTNYLEYIKREKNGALQAFFVNHVNNWVTRAYCDKVLRLSAATQDLPKSVVCNVHGVNPKFLMIGEKIAEERSRGEQAFSKGAYFLGKMVWAKGYRELIDLMAKHKSELGSFNLDVYGNGEDAVEVQRAAKKHDLNLNFLKGRDHADDALHKYKVFINPSISDVLCTATAEALAMGKFVVCADHPSNEFFRSFPNCLTYKTSEDFVSKVQEAMTKEPLPLTPEQMYNLSWEAATQRFMEYSDLDKILNNGEGGRKMRKSRSVPSFNEVVDGGLAFSHYVLTGNDFLRLCTGATPRTKDYDNQHCKDLNLVPPHVHKPIFGW

SEQ ID NO: 24 - protein, 797 aa, Arabidopsis thaliana
MCVVIGLKSWVMVLVVIFIRYVAQGKGILQSHQLIDEFLKTVKVDGTLEDLNKSPFMKVLQSAEEAIVLPPFVALAIRPRPGVREYVRVNVYELSVDHLTVSEYLRFKEELVNGHANGDYLLELDFEPFNATLPRPTRSSSIGNGVQFLNRHLSSIMFRNKESMEPLLEFLRTHKHDGRPMMLNDRIQNIPILQGALARAEEFLSKLPLATPYSEFEFELQGMGFERGWGDTAQKVSEMVHLLLDILQAPDPSVLETFLGRIPMVFNVVILSPHGYFGQANVLGLPDTGGQVVYILDQVRALENEMLLRIQKQGLEVIPKILIVTRLLPEAKGTTCNQRLERVSGTEHAHILRIPFRTEKGILRKWISRFDVWPYLETFAEDASNEISAELQGVPNLIIGNYSDGNLVASLLASKLGVIQCNIAHALEKTKYPESDIYWRNHEDKYHFSSQFTADLIAMNNADFIITSTYQEIAGSKNNVGQYESHTAFTMPGLYRVVHGIDVFDPKFNMVSPGADMTIYFPYSDKERRLTALHESIEELLFSAEQNDEHVGLLSDQSKPIIFSMARLDRVKNLTGLVECYAKNSKLRELANLVIVGGYIDENQSRDREEMAEIQKMHSLIEQYDLHGEFRWIAAQMNRARNGELYRYIADTKGVFVQPAFYEAFGLTVVESMTCALPTFATCHGGPAEIIENGVSGFHIDPYHPDQVAATLVSFFETCNTNPNHWVKISEGGLKRIYERYTWKKYSERLLTLAGVYAFWKHVSKLERRETRRYLEMFYSLKFRDLANSIPLATDEN

SEQ ID NO: 25 - protein, 391 aa, Arabidopsis thaliana
MATFAELVLSTSRCTCPCRSFTRKPLIRPPLSGLRLPGDTKPLFRSGLGRISVSRRFLTAVARAESDQLGDDDHSKGIDRIHNLQNVEDKQKKASQLKKRVIFGIGIGLPVGCVVLAGGWVFTVALASSVFIGSREYFELVRSRGIAKGMTPPPRYVSRVCSVICALMPILTLYFGNIDILVTSAAFVVAIALLVQRGSPRFAQLSSTMFGLFYCGYLPSFWVKLRCGLAAPALNTGIGRTWPILLGGQAHWTVGLVATLISFSGVIATDTFAFLGGKTFGRTPLTSISPKKTWEGTIVGLVGCIAITILLSKYLSWPQSLFSSVAFGFLNFFGSVFGDLTESMIKRDAGVKDSGSLIPGHGGILDRVDSYIFTGALAYSFIKTSLKLYGV

SEQ ID NO: 26 - DNA, 12 bp, Artificial sequence
  Consensus sequence
ggacacgtggcn

SEQ ID NO: 27 - DNA, 14 bp, Artificial sequence
  Consensus sequence
nnsgccacgtggcn

SEQ ID NO: 28 - DNA, 13 bp, Artificial sequence
  Consensus sequence
gntgacgtggcmn

SEQ ID NO: 29 - DNA, 12 bp, Artificial sequence
  Consensus sequence
nnnnncaccgnn

SEQ ID NO: 30 - DNA, 17 bp, Artificial sequence
  Consensus sequence
nnwtnnyacgtgkcmnk

SEQ ID NO: 31 - DNA, 13 bp, Artificial sequence
  Consensus sequence
kkntkacgtggnn

SEQ ID NO: 32 - DNA, 18 bp, Artificial sequence
  Consensus sequence
nttwccwaawnnggnaan

SEQ ID NO: 33 - DNA, 18 bp, Artificial sequence
  Consensus sequence
nttnccwwwwnnggwaan

SEQ ID NO: 34 - DNA, 20 bp, Artificial sequence
  Consensus sequence
nsttwctawawawrgnaany

SEQ ID NO: 35 - DNA, 16 bp, Artificial sequence
  Consensus sequence
tnccawawwtrgnaan

SEQ ID NO: 36 - DNA, 14 bp, Artificial sequence
  Consensus sequence
tnccawwwatagnw

SEQ ID NO: 37 - DNA, 16 bp, Artificial sequence
  Consensus sequence
tnccawwwatagnaan

SEQ ID NO: 38 - DNA, 11 bp, Artificial sequence
  Consensus sequence
nnagatctann

SEQ ID NO: 39 - DNA, 14 bp, Artificial sequence
  Consensus sequence
nytkngtggnggnm

SEQ ID NO: 40 - DNA, 12 bp, Artificial sequence
  Consensus sequence
nnrngnggtgnn

SEQ ID NO: 41 - DNA, 17 bp, Artificial sequence
  Consensus sequence
rcacrgwtcccraggnn

SEQ ID NO: 42 - DNA, 12 bp, Artificial sequence
  Consensus sequence
wyktgtcwcmyy

SEQ ID NO: 43 - DNA, 10 bp, Artificial sequence
  Consensus sequence
nnagatnykn

SEQ ID NO: 44 - DNA, 10 bp, Artificial sequence
  Consensus sequence
nnatctaaan

SEQ ID NO: 45 - DNA, 10 bp, Artificial sequence
  Consensus sequence
ncaattattn

SEQ ID NO: 46 - DNA, 10 bp, Artificial sequence
  Consensus sequence
ncaatyattn

SEQ ID NO: 47 - DNA, 11 bp, Artificial sequence
  Consensus sequence
gtaatgattrc

SEQ ID NO: 48 - DNA, 17 bp, Artificial sequence
  Consensus sequence
wwwtaataaatgyamnn

SEQ ID NO: 49 - DNA, 13 bp, Artificial sequence
  Consensus sequence
cracggtaggtgg

SEQ ID NO: 50 - DNA, 11 bp, Artificial sequence
  Consensus sequence
nngacrgttas

SEQ ID NO: 51 - DNA, 13 bp, Artificial sequence
  Consensus sequence
ngggggtaggtgs

SEQ ID NO: 52 - DNA, 13 bp, Artificial sequence
  Consensus sequence
nnaaaccctarmn

SEQ ID NO: 53 - DNA, 10 bp, Artificial sequence
  Consensus sequence
nwccgcgtna

SEQ ID NO: 54 - DNA, 12 bp, Artificial sequence
  Consensus sequence
nnmrgcccawyw

SEQ ID NO: 55 - DNA, 12 bp, Artificial sequence
  Consensus sequence
gatgacgtggcm

SEQ ID NO: 56 - DNA, 12 bp, Artificial sequence
  Consensus sequence
ggrtgctgacgt

SEQ ID NO: 57 - DNA, 12 bp, Artificial sequence
  Consensus sequence
grtgacgtggcc

SEQ ID NO: 58 - DNA, 12 bp, Artificial sequence
  Consensus sequence
grtgacgtgtac

SEQ ID NO: 59 - DNA, 10 bp, Artificial sequence
  Consensus sequence
nmnccaatnn

SEQ ID NO: 60 - DNA, 10 bp, Artificial sequence
  Consensus sequence
nnncaatnnn

SEQ ID NO: 61 - DNA, 15 bp, Artificial sequence
  Consensus sequence
yytygngagttgsnr

SEQ ID NO: 62 - DNA, 18 bp, Artificial sequence
  Consensus sequence
mnyttcmaacacctaann

SEQ ID NO: 63 - DNA, 15 bp, Artificial sequence
  Consensus sequence
anammnaaaatctnm

SEQ ID NO: 64 - DNA, 11 bp, Artificial sequence
  Consensus sequence
ngctcagcgcn

SEQ ID NO: 65 - DNA, 18 bp, Artificial sequence
  Consensus sequence
nnnngacgcgtgkcnynn

SEQ ID NO: 66 - DNA, 10 bp, Artificial sequence
  Consensus sequence
nncacgtgnn

SEQ ID NO: 67 - DNA, 13 bp, Artificial sequence
  Consensus sequence
wnnrccgacntnn

SEQ ID NO: 68 - DNA, 10 bp, Artificial sequence
  Consensus sequence
nnwaaagnnn

SEQ ID NO: 69 - DNA, 10 bp, Artificial sequence
  Consensus sequence
nnwaaagcnn

SEQ ID NO: 70 - DNA, 10 bp, Artificial sequence
  Consensus sequence
nnnaaagnnn

SEQ ID NO: 71 - DNA, 11 bp, Artificial sequence
  Consensus sequence
nacacnygncc

SEQ ID NO: 72 - DNA, 14 bp, Artificial sequence
  Consensus sequence
ntktttcccgcynn

SEQ ID NO: 73 - DNA, 10 bp, Artificial sequence
  Consensus sequence
gacacgtggm

SEQ ID NO: 74 - DNA, 15 bp, Artificial sequence
  Consensus sequence
nnanttgaccawnnn

SEQ ID NO: 75 - DNA, 12 bp, Artificial sequence
  Consensus sequence
aagagccgccwn

SEQ ID NO: 76 - DNA, 19 bp, Artificial sequence
  Consensus sequence
ccaatnannwnnngccacg

SEQ ID NO: 77 - DNA, 16 bp, Artificial sequence
  Consensus sequence
yyakagaaatnntnnm

SEQ ID NO: 78 - DNA, 25 bp, Artificial sequence
  Consensus sequence
satsagagagagagagagaknrgnn

SEQ ID NO: 79 - DNA, 13 bp, Artificial sequence
  Consensus sequence
ngkyngttatsnn

SEQ ID NO: 80 - DNA, 14 bp, Artificial sequence
  Consensus sequence
mswnatgaaranna

SEQ ID NO: 81 - DNA, 10 bp, Artificial sequence
  Consensus sequence
nangataagr

SEQ ID NO: 82 - DNA, 10 bp, Artificial sequence
  Consensus sequence
nnwsacgtgk

SEQ ID NO: 83 - DNA, 10 bp, Artificial sequence
  Consensus sequence
nnnagccgcc

SEQ ID NO: 84 - DNA, 12 bp, Artificial sequence
  Consensus sequence
gatgagtcatnn

SEQ ID NO: 85 - DNA, 12 bp, Artificial sequence
  Consensus sequence
tkaggtwaatnt

SEQ ID NO: 86 - DNA, 11 bp, Artificial sequence
  Consensus sequence
amngttacnnt

SEQ ID NO: 87 - DNA, 10 bp, Artificial sequence
  Consensus sequence
ncaatyatta

SEQ ID NO: 88 - DNA, 10 bp, Artificial sequence
  Consensus sequence
gccacgtsnc

SEQ ID NO: 89 - DNA, 16 bp, Artificial sequence
  Consensus sequence
snyacgtcannnntnn

SEQ ID NO: 90 - DNA, 14 bp, Artificial sequence
  Consensus sequence
anwttatttwatan

SEQ ID NO: 91 - DNA, 16 bp, Artificial sequence
  Consensus sequence
gtcancgatccgcgnn

SEQ ID NO: 92 - DNA, 15 bp, Artificial sequence
  Consensus sequence
ngaarmntmyagaay

SEQ ID NO: 93 - DNA, 12 bp, Artificial sequence
  Consensus sequence
nskcaccgccny

SEQ ID NO: 94 - DNA, 10 bp, Artificial sequence
  Consensus sequence
nnngtgacan

SEQ ID NO: 95 - DNA, 16 bp, Artificial sequence
  Consensus sequence
nwwrmgataagrttat

SEQ ID NO: 96 - DNA, 10 bp, Artificial sequence
  Consensus sequence
nacanntgny

SEQ ID NO: 97 - DNA, 12 bp, Artificial sequence
  Consensus sequence
nnttttgtcsyt

SEQ ID NO: 98 - DNA, 12 bp, Artificial sequence
  Consensus sequence
ngtgacaggtnn

SEQ ID NO: 99 - DNA, 26 bp, Artificial sequence
  Consensus sequence
tccatagccatgcawgctgaagaatg

SEQ ID NO: 100 - DNA, 12 bp, Artificial sequence
  Consensus sequence
rnccantgkktn

SEQ ID NO: 101 - DNA, 20 bp, Artificial sequence
  Consensus sequence
anwtwccatwtwtrgnaask

SEQ ID NO: 102 - DNA, 15 bp, Artificial sequence
  Consensus sequence
nntcyaacggyyanw

SEQ ID NO: 103 - DNA, 11 bp, Artificial sequence
  Consensus sequence
nwggtagktrn

SEQ ID NO: 104 - DNA, 13 bp, Artificial sequence
  Consensus sequence
nnaaaacsgttan

SEQ ID NO: 105 - DNA, 11 bp, Artificial sequence
  Consensus sequence
nnagttagtta

SEQ ID NO: 106 - DNA, 10 bp, Artificial sequence
  Consensus sequence
cnytatccnn

SEQ ID NO: 107 - DNA, 12 bp, Artificial sequence
  Consensus sequence
nnmacgtgnyna

SEQ ID NO: 108 - DNA, 10 bp, Artificial sequence
  Consensus sequence
naaaagatta

SEQ ID NO: 109 - DNA, 14 bp, Artificial sequence
  Consensus sequence
twttgtctcttnaw

SEQ ID NO: 110 - DNA, 11 bp, Artificial sequence
  Consensus sequence
gncaccyyyna

SEQ ID NO: 111 - DNA, 12 bp, Artificial sequence
  Consensus sequence
gggrtttgktgg

SEQ ID NO: 112 - DNA, 10 bp, Artificial sequence
  Consensus sequence
ccayrtcatc

SEQ ID NO: 113 - DNA, 12 bp, Artificial sequence
  Consensus sequence
ytccacgtcawn

SEQ ID NO: 114 - DNA, 16 bp, Artificial sequence
  Consensus sequence
nakwtsacrtgnmtra

SEQ ID NO: 115 - DNA, 20 bp, Artificial sequence
  Consensus sequence
wwangtaagwgmtkacgtmt

SEQ ID NO: 116 - DNA, 20 bp, Artificial sequence
  Consensus sequence
tgacgtaagcrmtkacgymn

SEQ ID NO: 117 - DNA, 12 bp, Artificial sequence
  Consensus sequence
nngcacgtgcnn

SEQ ID NO: 118 - DNA, 13 bp, Artificial sequence
  Consensus sequence
nnnacgtgkcgnn

SEQ ID NO: 119 - DNA, 16 bp, Artificial sequence
  Consensus sequence
nswsktatccatnymn

SEQ ID NO: 120 - DNA, 18 bp, Artificial sequence
  Consensus sequence
ntawwnsccgtccnwyan

SEQ ID NO: 121 - DNA, 11 bp, Artificial sequence
  Consensus sequence
ggtwggtgaga

SEQ ID NO: 122 - DNA, 13 bp, Artificial sequence
  Consensus sequence
gkggttkgtkrra

SEQ ID NO: 123 - DNA, 10 bp, Artificial sequence
  Consensus sequence
nwnwaaagng

SEQ ID NO: 124 - DNA, 17 bp, Artificial sequence
  Consensus sequence
tganrtgtaaagkkraw

SEQ ID NO: 125 - DNA, 10 bp, Artificial sequence
  Consensus sequence
nnggncccac

SEQ ID NO: 126 - DNA, 10 bp, Artificial sequence
  Consensus sequence
gtggycccnn

SEQ ID NO: 127 - DNA, 18 bp, Artificial sequence
  Consensus sequence
gkrggmcacgtgrmswck

SEQ ID NO: 128 - DNA, 10 bp, Artificial sequence
  Consensus sequence
nnnggtwggt

SEQ ID NO: 129 - DNA, 10 bp, Artificial sequence
  Consensus sequence
nncacctgnn

SEQ ID NO: 130 - DNA, 11 bp, Artificial sequence
  Consensus sequence
ngcaacakawn

SEQ ID NO: 131 - DNA, 10 bp, Artificial sequence
  Consensus sequence
snnacgtnrs

SEQ ID NO: 132 - DNA, 12 bp, Artificial sequence
  Consensus sequence
gccacstcarct

SEQ ID NO: 133 - DNA, 14 bp, Artificial sequence
  Consensus sequence
nnaaaccctaawnn

SEQ ID NO: 134 - DNA, 13 bp, Artificial sequence
  Consensus sequence
nkscatgcatgnn

SEQ ID NO: 135 - DNA, 16 bp, Artificial sequence
  Consensus sequence
tytcatggwawyawnw

SEQ ID NO: 136 - DNA, 14 bp, Artificial sequence
  Consensus sequence
knrtnrttaawwwn

SEQ ID NO: 137 - DNA, 13 bp, Artificial sequence
  Consensus sequence
nnytgtcacnnkn

SEQ ID NO: 138 - DNA, 14 bp, Artificial sequence
  Consensus sequence
kcyyaacccawcnt

SEQ ID NO: 139 - DNA, 11 bp, Artificial sequence
  Consensus sequence
nntttttrnyw

SEQ ID NO: 140 - DNA, 12 bp, Artificial sequence
  Consensus sequence
antactattwnn

SEQ ID NO: 141 - DNA, 10 bp, Artificial sequence
  Consensus sequence
cyatttwtrg

SEQ ID NO: 142 - DNA, 15 bp, Artificial sequence
  Consensus sequence
nnctaaacaattwnn

SEQ ID NO: 143 - DNA, 10 bp, Artificial sequence
  Consensus sequence
nnsacgtggn

SEQ ID NO: 144 - DNA, 11 bp, Artificial sequence
  Consensus sequence
gnatattccnn

SEQ ID NO: 145 - DNA, 22 bp, Artificial sequence
  Consensus sequence
gncgtaynnnrtacgtaacynn

SEQ ID NO: 146 - DNA, 14 bp, Artificial sequence
  Consensus sequence
nynmtataaatana

SEQ ID NO: 147 - DNA, 12 bp, Artificial sequence
  Consensus sequence
nctatawawann

SEQ ID NO: 148 - DNA, 20 bp, Artificial sequence
  Consensus sequence
nnargggyaaawnngtmawn

SEQ ID NO: 149 - DNA, 10 bp, Artificial sequence
  Consensus sequence
nnatgwayct

SEQ ID NO: 150 - DNA, 10 bp, Artificial sequence
  Consensus sequence
nntgacgtnn

SEQ ID NO: 151 - DNA, 16 bp, Artificial sequence
  Consensus sequence
awnnnkccacgtcann

SEQ ID NO: 152 - DNA, 16 bp, Artificial sequence
  Consensus sequence
aaartcccacatcgnn

SEQ ID NO: 153 - DNA, 14 bp, Artificial sequence
  Consensus sequence
nnnstttgacynnn

SEQ ID NO: 154 - DNA, 10 bp, Artificial sequence
  Consensus sequence
nnnnttaatg

SEQ ID NO: 155 - DNA, 12 bp, Artificial sequence
  Consensus sequence
ttgaccgagcnn

SEQ ID NO: 156 - DNA, 23 bp, Artificial sequence
  Consensus sequence
agcytwnamnncagtacacyamc

SEQ ID NO: 157 - DNA, 17 bp, Unknown
  Transcription factor binding site
aataaacatttagacac

SEQ ID NO: 158 - DNA, 11 bp, Unknown
  Transcription factor binding site
taaatgtttat

SEQ ID NO: 159 - DNA, 10 bp, Unknown
  Transcription factor binding site
natgaacata

SEQ ID NO: 160 - DNA, 13 bp, Unknown
  Transcription factor binding site
aacgttgtccctg

SEQ ID NO: 161 - DNA, 17 bp, Unknown
  Transcription factor binding site
tatcagatcccaacgtt

SEQ ID NO: 162 - DNA, 15 bp, Unknown
  Transcription factor binding site
tgacaccctatcaga

SEQ ID NO: 163 - DNA, 17 bp, Unknown
  Transcription factor binding site
actctttgacaccctat

SEQ ID NO: 164 - DNA, 13 bp, Unknown
  Transcription factor binding site
aatactctttgac

SEQ ID NO: 165 - DNA, 17 bp, Unknown
  Transcription factor binding site
tgaccgaaattgtccca

SEQ ID NO: 166 - DNA, 17 bp, Unknown
  Transcription factor binding site
ggtcatgagttgcaaat

SEQ ID NO: 167 - DNA, 17 bp, Unknown
  Transcription factor binding site
tgagttgcaaattcaag

SEQ ID NO: 168 - DNA, 21 bp, Unknown
  Transcription factor binding site
atatacttgaatttgcaactc

SEQ ID NO: 169 - DNA, 17 bp, Unknown
  Transcription factor binding site
tgggatattcttcgaaa

SEQ ID NO: 170 - DNA, 17 bp, Unknown
  Transcription factor binding site
aagaatatcccatttga

SEQ ID NO: 171 - DNA, 11 bp, Unknown
  Transcription factor binding site
ctcattaatgt

SEQ ID NO: 172 - DNA, 11 bp, Unknown
  Transcription factor binding site
aacattaatga

SEQ ID NO: 173 - DNA, 11 bp, Unknown
  Transcription factor binding site
taatctaaaaa

SEQ ID NO: 174 - DNA, 17 bp, Unknown
  Transcription factor binding site
actatgataaaatttca

SEQ ID NO: 175 - DNA, 17 bp, Unknown
  Transcription factor binding site
tttggtgtaaaggctgt

SEQ ID NO: 176 - DNA, 17 bp, Unknown
  Transcription factor binding site
aaggctgtaaaaagaaa

SEQ ID NO: 177 - DNA, 17 bp, Unknown
  Transcription factor binding site
aaaaagaaattgttcac

SEQ ID NO: 178 - DNA, 21 bp, Unknown
  Transcription factor binding site
aaaagaaattgttcacttttg

SEQ ID NO: 179 - DNA, 17 bp, Unknown
  Transcription factor binding site
cgaaaacaaaagtgaac

SEQ ID NO: 180 - DNA, 23 bp, Unknown
  Transcription factor binding site
gccttcacataaacgaaaacaaa

SEQ ID NO: 181 - DNA, 11 bp, Unknown
  Transcription factor binding site
taaaagattgt

SEQ ID NO: 182 - DNA, 13 bp, Unknown
  Transcription factor binding site
aagactattttgg

SEQ ID NO: 183 - DNA, 17 bp, Unknown
  Transcription factor binding site
cattttatccaaaacac

SEQ ID NO: 184 - DNA, 17 bp, Unknown
  Transcription factor binding site
ttttggataaaatgata

SEQ ID NO: 185 - DNA, 11 bp, Unknown
  Transcription factor binding site
aaaatgatagt

SEQ ID NO: 186 - DNA, 15 bp, Unknown
  Transcription factor binding site
aatctataaaaacta

SEQ ID NO: 187 - DNA, 11 bp, Unknown
  Transcription factor binding site
gaatctataaa

SEQ ID NO: 188 - DNA, 15 bp, Unknown
  Transcription factor binding site
agcaaaagaatctat

SEQ ID NO: 189 - DNA, 21 bp, Unknown
  Transcription factor binding site
tatttcttctaaaagcaaaag

SEQ ID NO: 190 - DNA, 17 bp, Unknown
  Transcription factor binding site
ttcttctaaaagcaaaa

SEQ ID NO: 191 - DNA, 21 bp, Unknown
  Transcription factor binding site
ttttgcttttagaagaaatac

SEQ ID NO: 192 - DNA, 17 bp, Unknown
  Transcription factor binding site
aaatttcaaatgtattt

SEQ ID NO: 193 - DNA, 13 bp, Unknown
  Transcription factor binding site
tacatttgaaatt

SEQ ID NO: 194 - DNA, 17 bp, Unknown
  Transcription factor binding site
acatggaaaaaatttca

SEQ ID NO: 195 - DNA, 15 bp, Unknown
  Transcription factor binding site
caacatggaaaaaat

SEQ ID NO: 196 - DNA, 21 bp, Unknown
  Transcription factor binding site
ttatactcaacatggaaaaaa

SEQ ID NO: 197 - DNA, 21 bp, Unknown
  Transcription factor binding site
tttttccatgttgagtataaa

SEQ ID NO: 198 - DNA, 15 bp, Unknown
  Transcription factor binding site
tgaagatcatagaaa

SEQ ID NO: 199 - DNA, 17 bp, Unknown
  Transcription factor binding site
tcatagaaatattttaa

SEQ ID NO: 200 - DNA, 15 bp, Unknown
  Transcription factor binding site
agttaaaatatttct

SEQ ID NO: 201 - DNA, 17 bp, Unknown
  Transcription factor binding site
ttttcagttaaaatatt

SEQ ID NO: 202 - DNA, 15 bp, Unknown
  Transcription factor binding site
cagttataaatttgt

SEQ ID NO: 203 - DNA, 17 bp, Unknown
  Transcription factor binding site
ttgaatcagttataaat

SEQ ID NO: 204 - DNA, 15 bp, Unknown
  Transcription factor binding site
aaaaatggagagaat

SEQ ID NO: 205 - DNA, 21 bp, Unknown
  Transcription factor binding site
tctctccatttttatacctat

SEQ ID NO: 206 - DNA, 15 bp, Unknown
  Transcription factor binding site
taggtataaaaatgg

SEQ ID NO: 207 - DNA, 21 bp, Unknown
  Transcription factor binding site
ttacggttaaataggtataaa

SEQ ID NO: 208 - DNA, 17 bp, Unknown
  Transcription factor binding site
attacggttaaataggt

SEQ ID NO: 209 - DNA, 17 bp, Unknown
  Transcription factor binding site
cgattacggttaaatag

SEQ ID NO: 210 - DNA, 15 bp, Unknown
  Transcription factor binding site
attatataaaaaatc

SEQ ID NO: 211 - DNA, 15 bp, Unknown
  Transcription factor binding site
ggattatataaaaaa

SEQ ID NO: 212 - DNA, 17 bp, Unknown
  Transcription factor binding site
ccgttggttaattagga

SEQ ID NO: 213 - DNA, 17 bp, Unknown
  Transcription factor binding site
tgccgttggttaattag

SEQ ID NO: 214 - DNA, 15 bp, Unknown
  Transcription factor binding site
taaccaacggcatgt

SEQ ID NO: 215 - DNA, 17 bp, Unknown
  Transcription factor binding site
ttaattatccaatacat

SEQ ID NO: 216 - DNA, 10 bp, Unknown
  Transcription factor binding site
natccaatac

SEQ ID NO: 217 - DNA, 17 bp, Unknown
  Transcription factor binding site
tattggataattaaccg

SEQ ID NO: 218 - DNA, 11 bp, Unknown
  Transcription factor binding site
tggataattaa

SEQ ID NO: 219 - DNA, 17 bp, Unknown
  Transcription factor binding site
tgatcggttaattatcc

SEQ ID NO: 220 - DNA, 17 bp, Unknown
  Transcription factor binding site
gttgatcggttaattat

SEQ ID NO: 221 - DNA, 17 bp, Unknown
  Transcription factor binding site
gggtgagagttgatcgg

SEQ ID NO: 222 - DNA, 21 bp, Unknown
  Transcription factor binding site
tgattctattaggggtgagag

SEQ ID NO: 223 - DNA, 17 bp, Unknown
  Transcription factor binding site
ggtcatatccatcgttt

SEQ ID NO: 224 - DNA, 15 bp, Unknown
  Transcription factor binding site
aaaaattaaaacgat

SEQ ID NO: 225 - DNA, 11 bp, Unknown
  Transcription factor binding site
gccaccattca

SEQ ID NO: 226 - DNA, 17 bp, Unknown
  Transcription factor binding site
atattcacatccctaaa

SEQ ID NO: 227 - DNA, 15 bp, Unknown
  Transcription factor binding site
atatgaacggccaag

SEQ ID NO: 228 - DNA, 21 bp, Unknown
  Transcription factor binding site
tcttgctttaatttggattat

SEQ ID NO: 229 - DNA, 17 bp, Unknown
  Transcription factor binding site
tccaaattaaagcaaga

SEQ ID NO: 230 - DNA, 17 bp, Unknown
  Transcription factor binding site
agtaagataatccaaat

SEQ ID NO: 231 - DNA, 13 bp, Unknown
  Transcription factor binding site
tacatttggatta

SEQ ID NO: 232 - DNA, 17 bp, Unknown
  Transcription factor binding site
aatgtacacttgtcatt

SEQ ID NO: 233 - DNA, 11 bp, Unknown
  Transcription factor binding site
tacacttgtca

SEQ ID NO: 234 - DNA, 13 bp, Unknown
  Transcription factor binding site
tacacttgtcatt

SEQ ID NO: 235 - DNA, 21 bp, Unknown
  Transcription factor binding site
ttttactaattttggcaatga

SEQ ID NO: 236 - DNA, 21 bp, Unknown
  Transcription factor binding site
cattgccaaaattagtaaaat

SEQ ID NO: 237 - DNA, 11 bp, Unknown
  Transcription factor binding site
cacattattaa

SEQ ID NO: 238 - DNA, 17 bp, Unknown
  Transcription factor binding site
cacattattaaaatacc

SEQ ID NO: 239 - DNA, 27 bp, Unknown
  Transcription factor binding site
gtattattcatgcaaatgcagccaata

SEQ ID NO: 240 - DNA, 15 bp, Unknown
  Transcription factor binding site
ttgcatgaataatac

SEQ ID NO: 241 - DNA, 21 bp, Unknown
  Transcription factor binding site
ataatactacgtgtaagccca

SEQ ID NO: 242 - DNA, 17 bp, Unknown
  Transcription factor binding site
taatactacgtgtaagc

SEQ ID NO: 243 - DNA, 13 bp, Unknown
  Transcription factor binding site
gtaagcccaaaag

SEQ ID NO: 244 - DNA, 21 bp, Unknown
  Transcription factor binding site
tgggctacacgtgggttcttt

SEQ ID NO: 245 - DNA, 21 bp, Unknown
  Transcription factor binding site
aagaacccacgtgtagcccat

SEQ ID NO: 246 - DNA, 17 bp, Unknown
  Transcription factor binding site
gggctacacgtgggttc

SEQ ID NO: 247 - DNA, 27 bp, Unknown
  Transcription factor binding site
gtgtagcccatgcaaagttaacactca

SEQ ID NO: 248 - DNA, 17 bp, Unknown
  Transcription factor binding site
gcccatgcaaagttaac

SEQ ID NO: 249 - DNA, 23 bp, Unknown
  Transcription factor binding site
accccattcctcagtctccacta

SEQ ID NO: 250 - DNA, 19 bp, Unknown
  Transcription factor binding site
ccattcctcagtctccact

SEQ ID NO: 251 - DNA, 15 bp, Unknown
  Transcription factor binding site
ccactatataaaccc

SEQ ID NO: 252 - DNA, 15 bp, Unknown
  Transcription factor binding site
actatataaacccac

SEQ ID NO: 253 - DNA, 15 bp, Unknown
  Transcription factor binding site
tataaacccaccatc

SEQ ID NO: 254 - DNA, 15 bp, Unknown
  Transcription factor binding site
gtgggtttggtgaga

SEQ ID NO: 255 - DNA, 15 bp, Unknown
  Transcription factor binding site
accaaacccaccaca

SEQ ID NO: 256 - DNA, 15 bp, Unknown
  Transcription factor binding site
gtgtggtgggtttgg

SEQ ID NO: 257 - DNA, 15 bp, Unknown
  Transcription factor binding site
gttgtgtggtgggtt

SEQ ID NO: 258 - DNA, 17 bp, Unknown
  Transcription factor binding site
agttgtgagttgtgtgg

SEQ ID NO: 259 - DNA, 17 bp, Unknown
  Transcription factor binding site
gagagtgagttgtgagt

SEQ ID NO: 260 - DNA, 10 bp, Unknown
  Transcription factor binding site
natccaatcc

SEQ ID NO: 261 - DNA, 17 bp, Unknown
  Transcription factor binding site
ataggtgtaaaatccaa

SEQ ID NO: 262 - DNA, 13 bp, Unknown
  Transcription factor binding site
aagtttgtccctc

SEQ ID NO: 263 - DNA, 11 bp, Unknown
  Transcription factor binding site
aaatctacatt

SEQ ID NO: 264 - DNA, 15 bp, Unknown
  Transcription factor binding site
gcttgaaaaatctac

SEQ ID NO: 265 - DNA, 19 bp, Unknown
  Transcription factor binding site
agcatcaaacacaagaatc

SEQ ID NO: 266 - DNA, 11 bp, Unknown
  Transcription factor binding site
taaattaatgt

SEQ ID NO: 267 - DNA, 17 bp, Unknown
  Transcription factor binding site
tttgatattcctaacat

SEQ ID NO: 268 - DNA, 10 bp, Unknown
  Transcription factor binding site
natccaatat

SEQ ID NO: 269 - DNA, 15 bp, Unknown
  Transcription factor binding site
ccaatataaaatcat

SEQ ID NO: 270 - DNA, 17 bp, Unknown
  Transcription factor binding site
tattgggtaaaagaaag

SEQ ID NO: 271 - DNA, 10 bp, Unknown
  Transcription factor binding site
nacccaataa

SEQ ID NO: 272 - DNA, 17 bp, Unknown
  Transcription factor binding site
cttaatggaagaagcaa

SEQ ID NO: 273 - DNA, 11 bp, Unknown
  Transcription factor binding site
atgcttaatgg

SEQ ID NO: 274 - DNA, 15 bp, Unknown
  Transcription factor binding site
agcatataaacatca

SEQ ID NO: 275 - DNA, 11 bp, Unknown
  Transcription factor binding site
taaaagatact

SEQ ID NO: 276 - DNA, 15 bp, Unknown
  Transcription factor binding site
tgctaaactattggt

SEQ ID NO: 277 - DNA, 17 bp, Unknown
  Transcription factor binding site
caaagtctaaagcataa

SEQ ID NO: 278 - DNA, 11 bp, Unknown
  Transcription factor binding site
agcataattaa

SEQ ID NO: 279 - DNA, 17 bp, Unknown
  Transcription factor binding site
gcataattaaagcatca

SEQ ID NO: 280 - DNA, 21 bp, Unknown
  Transcription factor binding site
ataattaaagcatcacatgtg

SEQ ID NO: 281 - DNA, 13 bp, Unknown
  Transcription factor binding site
cacatgtgatgct

SEQ ID NO: 282 - DNA, 15 bp, Unknown
  Transcription factor binding site
atttatgaaaaaaag

SEQ ID NO: 283 - DNA, 11 bp, Unknown
  Transcription factor binding site
aaaaagattaa

SEQ ID NO: 284 - DNA, 11 bp, Unknown
  Transcription factor binding site
aatcttaatct

SEQ ID NO: 285 - DNA, 11 bp, Unknown
  Transcription factor binding site
gctattattcg

SEQ ID NO: 286 - DNA, 21 bp, Unknown
  Transcription factor binding site
tgatgtactagaggacatttt

SEQ ID NO: 287 - DNA, 11 bp, Unknown
  Transcription factor binding site
aaaaagtttga

SEQ ID NO: 288 - DNA, 21 bp, Unknown
  Transcription factor binding site
taaggggaatcaatggaaaaa

SEQ ID NO: 289 - DNA, 13 bp, Unknown
  Transcription factor binding site
ttccattgattcc

SEQ ID NO: 290 - DNA, 17 bp, Unknown
  Transcription factor binding site
tcatggataaggggaat

SEQ ID NO: 291 - DNA, 17 bp, Unknown
  Transcription factor binding site
tttcatggataagggga

SEQ ID NO: 292 - DNA, 17 bp, Unknown
  Transcription factor binding site
ccccttatccatgaaaa

SEQ ID NO: 293 - DNA, 17 bp, Unknown
  Transcription factor binding site
tttttttcatggataag

SEQ ID NO: 294 - DNA, 15 bp, Unknown
  Transcription factor binding site
atccatgaaaaaaat

SEQ ID NO: 295 - DNA, 15 bp, Unknown
  Transcription factor binding site
aaataaacaaattct

SEQ ID NO: 296 - DNA, 15 bp, Unknown
  Transcription factor binding site
ttttgtgtcttaaga

SEQ ID NO: 297 - DNA, 21 bp, Unknown
  Transcription factor binding site
tggggccatttttttgtgtct

SEQ ID NO: 298 - DNA, 13 bp, Unknown
  Transcription factor binding site
aatggccccacat

SEQ ID NO: 299 - DNA, 17 bp, Unknown
  Transcription factor binding site
atggccccacatccttt

SEQ ID NO: 300 - DNA, 17 bp, Unknown
  Transcription factor binding site
cctagtttgtttgaatt

SEQ ID NO: 301 - DNA, 13 bp, Unknown
  Transcription factor binding site
cgaggcccactaa

SEQ ID NO: 302 - DNA, 15 bp, Unknown
  Transcription factor binding site
tgttaaatcattgat

SEQ ID NO: 303 - DNA, 11 bp, Unknown
  Transcription factor binding site
tcaatgattta

SEQ ID NO: 304 - DNA, 11 bp, Unknown
  Transcription factor binding site
taaatcattga

SEQ ID NO: 305 - DNA, 15 bp, Unknown
  Transcription factor binding site
aaaaatgaatagttt

SEQ ID NO: 306 - DNA, 15 bp, Unknown
  Transcription factor binding site
aattaaactattcat

SEQ ID NO: 307 - DNA, 17 bp, Unknown
  Transcription factor binding site
attggaattaaactatt

SEQ ID NO: 308 - DNA, 17 bp, Unknown
  Transcription factor binding site
tctcgtgagtcatattc

SEQ ID NO: 309 - DNA, 15 bp, Unknown
  Transcription factor binding site
accatataaacctca

SEQ ID NO: 310 - DNA, 17 bp, Unknown
  Transcription factor binding site
tagaatgagtgatgagg

SEQ ID NO: 311 - DNA, 21 bp, Unknown
  Transcription factor binding site
tcattctatttttttaagtgc

SEQ ID NO: 312 - DNA, 17 bp, Unknown
  Transcription factor binding site
tttgcacttaaaaaaat

SEQ ID NO: 313 - DNA, 17 bp, Unknown
  Transcription factor binding site
tttttttaagtgcaaag

SEQ ID NO: 314 - DNA, 17 bp, Unknown
  Transcription factor binding site
ttaagtgcaaagcttca

SEQ ID NO: 315 - DNA, 10 bp, Unknown
  Transcription factor binding site
natgaagctt

SEQ ID NO: 316 - DNA, 15 bp, Unknown
  Transcription factor binding site
agaaccttcttgaac

SEQ ID NO: 317 - DNA, 17 bp, Unknown
  Transcription factor binding site
tgaacttagttatctct

SEQ ID NO: 318 - DNA, 17 bp, Unknown
  Transcription factor binding site
agcaatatgtcatcaac

SEQ ID NO: 319 - DNA, 15 bp, Unknown
  Transcription factor binding site
aacatataaacatgt

SEQ ID NO: 320 - DNA, 17 bp, Unknown
  Transcription factor binding site
agtaatgttactggtgg

SEQ ID NO: 321 - DNA, 10 bp, Unknown
  Transcription factor binding site
natgaacttg

SEQ ID NO: 322 - DNA, 17 bp, Unknown
  Transcription factor binding site
ctttgttagtttctgga

SEQ ID NO: 323 - DNA, 17 bp, Unknown
  Transcription factor binding site
tcaactttgttagtttc

SEQ ID NO: 324 - DNA, 13 bp, Unknown
  Transcription factor binding site
ctttttgtctttc

SEQ ID NO: 325 - DNA, 21 bp, Unknown
  Transcription factor binding site
attaaatgacggctgcaaaat

SEQ ID NO: 326 - DNA, 11 bp, Unknown
  Transcription factor binding site
gtaatgaatac

SEQ ID NO: 327 - DNA, 17 bp, Unknown
  Transcription factor binding site
aggtaggtcaatgtatt

SEQ ID NO: 328 - DNA, 17 bp, Unknown
  Transcription factor binding site
atacattgacctaccta

SEQ ID NO: 329 - DNA, 15 bp, Unknown
  Transcription factor binding site
agtaggtaggtcaat

SEQ ID NO: 330 - DNA, 21 bp, Unknown
  Transcription factor binding site
ctaggctatttatacacaata

SEQ ID NO: 331 - DNA, 15 bp, Unknown
  Transcription factor binding site
tgtgtataaatagcc

SEQ ID NO: 332 - DNA, 15 bp, Unknown
  Transcription factor binding site
ttataccctaatatt

SEQ ID NO: 333 - DNA, 15 bp, Unknown
  Transcription factor binding site
ttaatattttattat

SEQ ID NO: 334 - DNA, 17 bp, Unknown
  Transcription factor binding site
tcttattgactaagtct

SEQ ID NO: 335 - DNA, 15 bp, Unknown
  Transcription factor binding site
aaaatataaattatt

SEQ ID NO: 336 - DNA, 17 bp, Unknown
  Transcription factor binding site
tgttggaaataatttat

SEQ ID NO: 337 - DNA, 11 bp, Unknown
  Transcription factor binding site
taaattatttc

SEQ ID NO: 338 - DNA, 11 bp, Unknown
  Transcription factor binding site
gaaataattta

SEQ ID NO: 339 - DNA, 11 bp, Unknown
  Transcription factor binding site
caaattattgt

SEQ ID NO: 340 - DNA, 11 bp, Unknown
  Transcription factor binding site
acaataatttg

SEQ ID NO: 341 - DNA, 13 bp, Unknown
  Transcription factor binding site
taatttgtctcaa

SEQ ID NO: 342 - DNA, 13 bp, Unknown
  Transcription factor binding site
atttgtctcaaat

SEQ ID NO: 343 - DNA, 17 bp, Unknown
  Transcription factor binding site
agaggtgcaaaagttaa

SEQ ID NO: 344 - DNA, 17 bp, Unknown
  Transcription factor binding site
aagagtgcaaagtaaaa

SEQ ID NO: 345 - DNA, 17 bp, Unknown
  Transcription factor binding site
aatattgttattatatt

SEQ ID NO: 346 - DNA, 23 bp, Unknown
  Transcription factor binding site
tccctcaactgtacgtagctcct

SEQ ID NO: 347 - DNA, 17 bp, Unknown
  Transcription factor binding site
tgcagtgtaaagatttg

SEQ ID NO: 348 - DNA, 17 bp, Unknown
  Transcription factor binding site
ttgaagataaaggttca

SEQ ID NO: 349 - DNA, 17 bp, Unknown
  Transcription factor binding site
atgtgttagttcctgaa

SEQ ID NO: 350 - DNA, 21 bp, Unknown
  Transcription factor binding site
aactcccctatttggcatgta

SEQ ID NO: 351 - DNA, 21 bp, Unknown
  Transcription factor binding site
acatgccaaataggggagtta

SEQ ID NO: 352 - DNA, 17 bp, Unknown
  Transcription factor binding site
ctatcttgaccctttct

SEQ ID NO: 353 - DNA, 21 bp, Unknown
  Transcription factor binding site
aaagggtcaagatagtgatgt

SEQ ID NO: 354 - DNA, 17 bp, Unknown
  Transcription factor binding site
tcaagatagtgatgtgc

SEQ ID NO: 355 - DNA, 19 bp, Unknown
  Transcription factor binding site
cccattatgaaggatcacg

SEQ ID NO: 356 - DNA, 21 bp, Unknown
  Transcription factor binding site
tcatactacaaagagatcatg

SEQ ID NO: 357 - DNA, 27 bp, Unknown
  Transcription factor binding site
aaagagatcatgcataaaaccaactag

SEQ ID NO: 358 - DNA, 17 bp, Unknown
  Transcription factor binding site
tctagttggttttatgc

SEQ ID NO: 359 - DNA, 21 bp, Unknown
  Transcription factor binding site
atacttgacagttgacttcta

SEQ ID NO: 360 - DNA, 17 bp, Unknown
  Transcription factor binding site
acttgacagttgacttc

SEQ ID NO: 361 - DNA, 21 bp, Unknown
  Transcription factor binding site
aactgtcaagtatgacggctg

SEQ ID NO: 362 - DNA, 21 bp, Unknown
  Transcription factor binding site
caagtatgacggctgacaatt

SEQ ID NO: 363 - DNA, 17 bp, Unknown
  Transcription factor binding site
ggtggacggttaattgt

SEQ ID NO: 364 - DNA, 19 bp, Unknown
  Transcription factor binding site
caattaaccgtccaccaaa

SEQ ID NO: 365 - DNA, 15 bp, Unknown
  Transcription factor binding site
ggaagatttggtgga

SEQ ID NO: 366 - DNA, 17 bp, Unknown
  Transcription factor binding site
atgtatggatataagaa

SEQ ID NO: 367 - DNA, 17 bp, Unknown
  Transcription factor binding site
tcttatatccatacatt

SEQ ID NO: 368 - DNA, 17 bp, Unknown
  Transcription factor binding site
atgtcatcaatgtatgg

SEQ ID NO: 369 - DNA, 17 bp, Unknown
  Transcription factor binding site
tcaataatgtcatcaat

SEQ ID NO: 370 - DNA, 17 bp, Unknown
  Transcription factor binding site
ttgatgacattattgat

SEQ ID NO: 371 - DNA, 15 bp, Unknown
  Transcription factor binding site
gaaaaccccaatctc

SEQ ID NO: 372 - DNA, 21 bp, Unknown
  Transcription factor binding site
cccatccaagtaaagctgtaa

SEQ ID NO: 373 - DNA, 17 bp, Unknown
  Transcription factor binding site
atccaagtaaagctgta

SEQ ID NO: 374 - DNA, 15 bp, Unknown
  Transcription factor binding site
ctatgactcttcacc

SEQ ID NO: 375 - DNA, 17 bp, Unknown
  Transcription factor binding site
ggtgaagagtcatagcc

SEQ ID NO: 376 - DNA, 17 bp, Unknown
  Transcription factor binding site
cagacgcagttagatac

SEQ ID NO: 377 - DNA, 11 bp, Unknown
  Transcription factor binding site
gtatctaactg

SEQ ID NO: 378 - DNA, 10 bp, Unknown
  Transcription factor binding site
natgaacttg

SEQ ID NO: 379 - DNA, 11 bp, Unknown
  Transcription factor binding site
aacaccctcaa

SEQ ID NO: 380 - DNA, 13 bp, Unknown
  Transcription factor binding site
tcgtgtcacaaaa

SEQ ID NO: 381 - DNA, 17 bp, Unknown
  Transcription factor binding site
tctctacagttagaaat

SEQ ID NO: 382 - DNA, 17 bp, Unknown
  Transcription factor binding site
gtagaacacttggctgt

SEQ ID NO: 383 - DNA, 11 bp, Unknown
  Transcription factor binding site
ttatctatacc

SEQ ID NO: 384 - DNA, 17 bp, Unknown
  Transcription factor binding site
gtatagataaatgaatg

SEQ ID NO: 385 - DNA, 17 bp, Unknown
  Transcription factor binding site
tatagataaatgaatga

SEQ ID NO: 386 - DNA, 21 bp, Unknown
  Transcription factor binding site
atcataaatcgtcattcattt

SEQ ID NO: 387 - DNA, 17 bp, Unknown
  Transcription factor binding site
atatgggttaccctatt

SEQ ID NO: 388 - DNA, 11 bp, Unknown
  Transcription factor binding site
gtatctagaag

SEQ ID NO: 389 - DNA, 13 bp, Unknown
  Transcription factor binding site
gtatttgtctccc

SEQ ID NO: 390 - DNA, 13 bp, Unknown
  Transcription factor binding site
atttgtctccctt

SEQ ID NO: 391 - DNA, 17 bp, Unknown
  Transcription factor binding site
gtggagataaggcaaga

SEQ ID NO: 392 - DNA, 11 bp, Unknown
  Transcription factor binding site
aaaatcattgt

SEQ ID NO: 393 - DNA, 11 bp, Unknown
  Transcription factor binding site
acaatgatttt

SEQ ID NO: 394 - DNA, 13 bp, Unknown
  Transcription factor binding site
ggttttgtcaaca

SEQ ID NO: 395 - DNA, 11 bp, Unknown
  Transcription factor binding site
acaatcattga

SEQ ID NO: 396 - DNA, 11 bp, Unknown
  Transcription factor binding site
tcaatgattgt

SEQ ID NO: 397 - DNA, 17 bp, Unknown
  Transcription factor binding site
gtttaggtaaagttgaa

SEQ ID NO: 398 - DNA, 21 bp, Unknown
  Transcription factor binding site
taatggacaatcaagtttcaa

SEQ ID NO: 399 - DNA, 17 bp, Unknown
  Transcription factor binding site
cactgactgttgaagac

SEQ ID NO: 400 - DNA, 19 bp, Unknown
  Transcription factor binding site
gcctaacgcgtctcgcata

SEQ ID NO: 401 - DNA, 11 bp, Unknown
  Transcription factor binding site
taacgcgtctc

SEQ ID NO: 402 - DNA, 11 bp, Unknown
  Transcription factor binding site
agacgcgttag

SEQ ID NO: 403 - DNA, 17 bp, Unknown
  Transcription factor binding site
aagacgtagttaggatg

SEQ ID NO: 404 - DNA, 17 bp, Unknown
  Transcription factor binding site
gttaggatgtcatcata

SEQ ID NO: 405 - DNA, 15 bp, Unknown
  Transcription factor binding site
tacgaaacaaattat

SEQ ID NO: 406 - DNA, 15 bp, Unknown
  Transcription factor binding site
tacatataaaaatac

SEQ ID NO: 407 - DNA, 15 bp, Unknown
  Transcription factor binding site
tacatataaaaactg

SEQ ID NO: 408 - DNA, 15 bp, Unknown
  Transcription factor binding site
aatatatacatataa

SEQ ID NO: 409 - DNA, 17 bp, Unknown
  Transcription factor binding site
gaatcgataaaaaacta

SEQ ID NO: 410 - DNA, 17 bp, Unknown
  Transcription factor binding site
tcgattcagttatttga

SEQ ID NO: 411 - DNA, 13 bp, Unknown
  Transcription factor binding site
attactttttctc

SEQ ID NO: 412 - DNA, 13 bp, Unknown
  Transcription factor binding site
ctttttgtctgca

SEQ ID NO: 413 - DNA, 21 bp, Unknown
  Transcription factor binding site
cttttccactttttgtctgca

SEQ ID NO: 414 - DNA, 17 bp, Unknown
  Transcription factor binding site
gcagacaaaaagtggaa

SEQ ID NO: 415 - DNA, 15 bp, Unknown
  Transcription factor binding site
gattgtcttttccac

SEQ ID NO: 416 - DNA, 15 bp, Unknown
  Transcription factor binding site
gaaaagacaatctga

SEQ ID NO: 417 - DNA, 21 bp, Unknown
  Transcription factor binding site
aatttccaatttttgaaattt

SEQ ID NO: 418 - DNA, 15 bp, Unknown
  Transcription factor binding site
taattataaaaaaat

SEQ ID NO: 419 - DNA, 11 bp, Unknown
  Transcription factor binding site
tttataattat

SEQ ID NO: 420 - DNA, 11 bp, Unknown
  Transcription factor binding site
ctgataattat

SEQ ID NO: 421 - DNA, 21 bp, Unknown
  Transcription factor binding site
tccgataaaaacatacatgta

SEQ ID NO: 422 - DNA, 10 bp, Unknown
  Transcription factor binding site
natgtatgtt

SEQ ID NO: 423 - DNA, 11 bp, Unknown
  Transcription factor binding site
cgatctataca

SEQ ID NO: 424 - DNA, 15 bp, Unknown
  Transcription factor binding site
tgaaagtactagaaa

SEQ ID NO: 425 - DNA, 15 bp, Unknown
  Transcription factor binding site
ttttaaaaaattaca

SEQ ID NO: 426 - DNA, 21 bp, Unknown
  Transcription factor binding site
ttttttaaaaatttacgataa

SEQ ID NO: 427 - DNA, 17 bp, Unknown
  Transcription factor binding site
attatcgtaaattttta

SEQ ID NO: 428 - DNA, 17 bp, Unknown
  Transcription factor binding site
tttacgataatttacag

SEQ ID NO: 429 - DNA, 13 bp, Unknown
  Transcription factor binding site
aatactgtaaatt

SEQ ID NO: 430 - DNA, 17 bp, Unknown
  Transcription factor binding site
acagtatttaaaaaaaa

SEQ ID NO: 431 - DNA, 15 bp, Unknown
  Transcription factor binding site
agtatttaaaaaaaa

SEQ ID NO: 432 - DNA, 15 bp, Unknown
  Transcription factor binding site
taaaaaaaaatccaa

SEQ ID NO: 433 - DNA, 10 bp, Unknown
  Transcription factor binding site
natccaatct

SEQ ID NO: 434 - DNA, 21 bp, Unknown
  Transcription factor binding site
taaagggtataagaataaaag

SEQ ID NO: 435 - DNA, 17 bp, Unknown
  Transcription factor binding site
taagaataaaagcactc

SEQ ID NO: 436 - DNA, 17 bp, Unknown
  Transcription factor binding site
taagaataaaagcactc

SEQ ID NO: 437 - DNA, 21 bp, Unknown
  Transcription factor binding site
agggtgtgacgaaacctgcca

SEQ ID NO: 438 - DNA, 21 bp, Unknown
  Transcription factor binding site
ggcaggtttcgtcacacccta

SEQ ID NO: 439 - DNA, 15 bp, Unknown
  Transcription factor binding site
tcacaccctaagaac

SEQ ID NO: 440 - DNA, 15 bp, Unknown
  Transcription factor binding site
aacatccctaaatac

SEQ ID NO: 441 - DNA, 15 bp, Unknown
  Transcription factor binding site
cacatataaatattt

SEQ ID NO: 442 - DNA, 17 bp, Unknown
  Transcription factor binding site
ctgaatattaaatttca

SEQ ID NO: 443 - DNA, 17 bp, Unknown
  Transcription factor binding site
cagcttacttgattaaa

SEQ ID NO: 444 - DNA, 11 bp, Unknown
  Transcription factor binding site
gagtttaatca

SEQ ID NO: 445 - DNA, 17 bp, Unknown
  Transcription factor binding site
tattgggtcactatgga

SEQ ID NO: 446 - DNA, 10 bp, Unknown
  Transcription factor binding site
nacccaataa

SEQ ID NO: 447 - DNA, 21 bp, Unknown
  Transcription factor binding site
cccaataagtgctaactttta

SEQ ID NO: 448 - DNA, 21 bp, Unknown
  Transcription factor binding site
taaaggtaaagacagtaaaag

SEQ ID NO: 449 - DNA, 17 bp, Unknown
  Transcription factor binding site
taacatttaaaggtaaa

SEQ ID NO: 450 - DNA, 17 bp, Unknown
  Transcription factor binding site
gaaaaagaaatgcataa

SEQ ID NO: 451 - DNA, 15 bp, Unknown
  Transcription factor binding site
ctttttcctgcatct

SEQ ID NO: 452 - DNA, 13 bp, Unknown
  Transcription factor binding site
tatactattgaga

SEQ ID NO: 453 - DNA, 15 bp, Unknown
  Transcription factor binding site
tgataccctatatac

SEQ ID NO: 454 - DNA, 13 bp, Unknown
  Transcription factor binding site
atcactatttgat

SEQ ID NO: 455 - DNA, 17 bp, Unknown
  Transcription factor binding site
gtgattatccaaactta

SEQ ID NO: 456 - DNA, 13 bp, Unknown
  Transcription factor binding site
tttactattgaat

SEQ ID NO: 457 - DNA, 17 bp, Unknown
  Transcription factor binding site
gtgatacagttaaaatg

SEQ ID NO: 458 - DNA, 17 bp, Unknown
  Transcription factor binding site
gatacagttaaaatgac

SEQ ID NO: 459 - DNA, 21 bp, Unknown
  Transcription factor binding site
agggaccttaattagtagttt

SEQ ID NO: 460 - DNA, 15 bp, Unknown
  Transcription factor binding site
aagttatttttttag

SEQ ID NO: 461 - DNA, 15 bp, Unknown
  Transcription factor binding site
ttctaaaaaaataac

SEQ ID NO: 462 - DNA, 11 bp, Unknown
  Transcription factor binding site
tctttttatta

SEQ ID NO: 463 - DNA, 21 bp, Unknown
  Transcription factor binding site
agatagactcgtcattctttt

SEQ ID NO: 464 - DNA, 11 bp, Unknown
  Transcription factor binding site
ctatctaaatc

SEQ ID NO: 465 - DNA, 17 bp, Unknown
  Transcription factor binding site
ttacttgttaatatgat

SEQ ID NO: 466 - DNA, 21 bp, Unknown
  Transcription factor binding site
ggaggccaataattgtagtaa

SEQ ID NO: 467 - DNA, 11 bp, Unknown
  Transcription factor binding site
ccaataattgt

SEQ ID NO: 468 - DNA, 11 bp, Unknown
  Transcription factor binding site
acaattattgg

SEQ ID NO: 469 - DNA, 17 bp, Unknown
  Transcription factor binding site
gccgaggttaatatatg

SEQ ID NO: 470 - DNA, 21 bp, Unknown
  Transcription factor binding site
atatgctcaagacagtaaata

SEQ ID NO: 471 - DNA, 15 bp, Unknown
  Transcription factor binding site
cagtaaataatctaa

SEQ ID NO: 472 - DNA, 17 bp, Unknown
  Transcription factor binding site
ataatctaaatgaatta

SEQ ID NO: 473 - DNA, 11 bp, Unknown
  Transcription factor binding site
taatctaaatg

SEQ ID NO: 474 - DNA, 17 bp, Unknown
  Transcription factor binding site
tgatttgcaaagagtag

SEQ ID NO: 475 - DNA, 17 bp, Unknown
  Transcription factor binding site
aaagagtagatgcagag

SEQ ID NO: 476 - DNA, 17 bp, Unknown
  Transcription factor binding site
agagaactaaagatttg

SEQ ID NO: 477 - DNA, 10 bp, Unknown
  Transcription factor binding site
nagatttgct

SEQ ID NO: 478 - DNA, 21 bp, Unknown
  Transcription factor binding site
tcttatatacgtgtagcagca

SEQ ID NO: 479 - DNA, 11 bp, Unknown
  Transcription factor binding site
agcaacagata

SEQ ID NO: 480 - DNA, 21 bp, Unknown
  Transcription factor binding site
atattccacaaagagacagaa

SEQ ID NO: 481 - DNA, 15 bp, Unknown
  Transcription factor binding site
ttctgtctctttgtg

SEQ ID NO: 482 - DNA, 17 bp, Unknown
  Transcription factor binding site
atccatattccacaaag

SEQ ID NO: 483 - DNA, 17 bp, Unknown
  Transcription factor binding site
gtagatatccatattcc

SEQ ID NO: 484 - DNA, 11 bp, Unknown
  Transcription factor binding site
atatctactaa

SEQ ID NO: 485 - DNA, 11 bp, Unknown
  Transcription factor binding site
atgatgattag

SEQ ID NO: 486 - DNA, 13 bp, Unknown
  Transcription factor binding site
cacatatgatgtg

SEQ ID NO: 487 - DNA, 27 bp, Unknown
  Transcription factor binding site
ttcttcagcatgcatggctatggagtc

SEQ ID NO: 488 - DNA, 27 bp, Unknown
  Transcription factor binding site
tccatagccatgcatgctgaagaatgt

SEQ ID NO: 489 - DNA, 21 bp, Unknown
  Transcription factor binding site
acacgtgtgacagaacgtgtg

SEQ ID NO: 490 - DNA, 13 bp, Unknown
  Transcription factor binding site
ttctgtcacacgt

SEQ ID NO: 491 - DNA, 21 bp, Unknown
  Transcription factor binding site
agagtaacacgtgtgacagaa

SEQ ID NO: 492 - DNA, 21 bp, Unknown
  Transcription factor binding site
tctgtcacacgtgttactctc

SEQ ID NO: 493 - DNA, 17 bp, Unknown
  Transcription factor binding site
ctgtcacacgtgttact

SEQ ID NO: 494 - DNA, 17 bp, Unknown
  Transcription factor binding site
gagtaacacgtgtgaca

SEQ ID NO: 495 - DNA, 13 bp, Unknown
  Transcription factor binding site
cacacgtgttact

SEQ ID NO: 496 - DNA, 17 bp, Unknown
  Transcription factor binding site
acacgtgttactctctc

SEQ ID NO: 497 - DNA, 15 bp, Unknown
  Transcription factor binding site
ttcctataaatcacc

SEQ ID NO: 498 - DNA, 10 bp, Unknown
  Transcription factor binding site
nnnnncaccg

SEQ ID NO: 499 - DNA, 21 bp, Unknown
  Transcription factor binding site
aagtggtgaagtggagaagct

SEQ ID NO: 500 - DNA, 17 bp, Unknown
  Transcription factor binding site
ttctccacttcaccact

SEQ ID NO: 501 - DNA, 21 bp, Unknown
  Transcription factor binding site
aagtggtgaagtggtgaagtg

SEQ ID NO: 502 - DNA, 13 bp, Unknown
  Transcription factor binding site
tttactatcacag

SEQ ID NO: 503 - DNA, 17 bp, Unknown
  Transcription factor binding site
aaaaagaaatggtaact

SEQ ID NO: 504 - DNA, 15 bp, Unknown
  Transcription factor binding site
ctttttcctgcatct

SEQ ID NO: 505 - DNA, 13 bp, Unknown
  Transcription factor binding site
tatactattgaga

SEQ ID NO: 506 - DNA, 15 bp, Unknown
  Transcription factor binding site
tgataccctatatac

SEQ ID NO: 507 - DNA, 13 bp, Unknown
  Transcription factor binding site
atcactatttgat

SEQ ID NO: 508 - DNA, 17 bp, Unknown
  Transcription factor binding site
gtgattatccaaactta

SEQ ID NO: 509 - DNA, 13 bp, Unknown
  Transcription factor binding site
tttactattgaat

SEQ ID NO: 510 - DNA, 17 bp, Unknown
  Transcription factor binding site
gtgatacagttaaaatg

SEQ ID NO: 511 - DNA, 17 bp, Unknown
  Transcription factor binding site
gatacagttaaaatgac

SEQ ID NO: 512 - DNA, 21 bp, Unknown
  Transcription factor binding site
agggaccttaattagtagttt

SEQ ID NO: 513 - DNA, 15 bp, Unknown
  Transcription factor binding site
aagttattttcttag

SEQ ID NO: 514 - DNA, 11 bp, Unknown
  Transcription factor binding site
tctttttatta

SEQ ID NO: 515 - DNA, 21 bp, Unknown
  Transcription factor binding site
agatagactcgtcattctttt

SEQ ID NO: 516 - DNA, 11 bp, Unknown
  Transcription factor binding site
ctatctaaatc

SEQ ID NO: 517 - DNA, 17 bp, Unknown
  Transcription factor binding site
ttacttgttaatatgat

SEQ ID NO: 518 - DNA, 21 bp, Unknown
  Transcription factor binding site
ggaggccaataattgtagtaa

SEQ ID NO: 519 - DNA, 11 bp, Unknown
  Transcription factor binding site
ccaataattgt

SEQ ID NO: 520 - DNA, 11 bp, Unknown
  Transcription factor binding site
acaattattgg

SEQ ID NO: 521 - DNA, 17 bp, Unknown
  Transcription factor binding site
gccgaggttaatatatg

SEQ ID NO: 522 - DNA, 21 bp, Unknown
  Transcription factor binding site
atatgctcaagacagtaaata

SEQ ID NO: 523 - DNA, 15 bp, Unknown
  Transcription factor binding site
cagtaaataatctaa

SEQ ID NO: 524 - DNA, 17 bp, Unknown
  Transcription factor binding site
ataatctaaatgaatta

SEQ ID NO: 525 - DNA, 11 bp, Unknown
  Transcription factor binding site
taatctaaatg

SEQ ID NO: 526 - DNA, 17 bp, Unknown
  Transcription factor binding site
tgatttgcaaagagtag

SEQ ID NO: 527 - DNA, 17 bp, Unknown
  Transcription factor binding site
aaagagtagatgcagag

SEQ ID NO: 528 - DNA, 17 bp, Unknown
  Transcription factor binding site
agagaactaaagatttg

SEQ ID NO: 529 - DNA, 10 bp, Unknown
  Transcription factor binding site
nagatttgct

SEQ ID NO: 530 - DNA, 21 bp, Unknown
  Transcription factor binding site
tcttatatacgtgtagcagca

SEQ ID NO: 531 - DNA, 11 bp, Unknown
  Transcription factor binding site
agcaacagata

SEQ ID NO: 532 - DNA, 21 bp, Unknown
  Transcription factor binding site
atattccacaaagagacagaa

SEQ ID NO: 533 - DNA, 15 bp, Unknown
  Transcription factor binding site
ttctgtctctttgtg

SEQ ID NO: 534 - DNA, 17 bp, Unknown
  Transcription factor binding site
atccatattccacaaag

SEQ ID NO: 535 - DNA, 17 bp, Unknown
  Transcription factor binding site
gtagatatccatattcc

SEQ ID NO: 536 - DNA, 11 bp, Unknown
  Transcription factor binding site
atatctactaa

SEQ ID NO: 537 - DNA, 11 bp, Unknown
  Transcription factor binding site
atgatgattag

SEQ ID NO: 538 - DNA, 13 bp, Unknown
  Transcription factor binding site
cacatatgatgtg

SEQ ID NO: 539 - DNA, 27 bp, Unknown
  Transcription factor binding site
ttcttcagcatgcatggctatggagtc

SEQ ID NO: 540 - DNA, 27 bp, Unknown
  Transcription factor binding site
tccatagccatgcatgctgaagaatgt

SEQ ID NO: 541 - DNA, 21 bp, Unknown
  Transcription factor binding site
acacgtgtgacagaacgtgtg

SEQ ID NO: 542 - DNA, 13 bp, Unknown
  Transcription factor binding site
ttctgtcacacgt

SEQ ID NO: 543 - DNA, 21 bp, Unknown
  Transcription factor binding site
agagtaacacgtgtgacagaa

SEQ ID NO: 544 - DNA, 21 bp, Unknown
  Transcription factor binding site
tctgtcacacgtgttactctc

SEQ ID NO: 545 - DNA, 17 bp, Unknown
  Transcription factor binding site
ctgtcacacgtgttact

SEQ ID NO: 546 - DNA, 17 bp, Unknown
  Transcription factor binding site
gagtaacacgtgtgaca

SEQ ID NO: 547 - DNA, 13 bp, Unknown
  Transcription factor binding site
cacacgtgttact

SEQ ID NO: 548 - DNA, 17 bp, Unknown
  Transcription factor binding site
acacgtgttactctctc

SEQ ID NO: 549 - DNA, 15 bp, Unknown
  Transcription factor binding site
ttcctataaatcacc

SEQ ID NO: 550 - DNA, 10 bp, Unknown
  Transcription factor binding site
nnnnncaccg

SEQ ID NO: 551 - DNA, 21 bp, Unknown
  Transcription factor binding site
aagtggtgaagtggagaagct

SEQ ID NO: 552 - DNA, 17 bp, Unknown
  Transcription factor binding site
ttctccacttcaccact

SEQ ID NO: 553 - DNA, 21 bp, Unknown
  Transcription factor binding site
aagtggtgaagtggtgaagtg

SEQ ID NO: 554 - DNA, 13 bp, Unknown
  Transcription factor binding site
tttactatcacag

SEQ ID NO: 555 - DNA, 17 bp, Unknown
  Transcription factor binding site
aaatttacacattgcca

SEQ ID NO: 556 - DNA, 15 bp, Unknown
  Transcription factor binding site
ctaaacccttgtaat

SEQ ID NO: 557 - DNA, 11 bp, Unknown
  Transcription factor binding site
tgtttttgttt

SEQ ID NO: 558 - DNA, 13 bp, Unknown
  Transcription factor binding site
tttactatgtgtg

SEQ ID NO: 559 - DNA, 17 bp, Unknown
  Transcription factor binding site
ctatgtgtgttatgtat

SEQ ID NO: 560 - DNA, 21 bp, Unknown
  Transcription factor binding site
tagtaccaaatataaaaattt

SEQ ID NO: 561 - DNA, 15 bp, Unknown
  Transcription factor binding site
caaatataaaaattt

SEQ ID NO: 562 - DNA, 15 bp, Unknown
  Transcription factor binding site
gtgttataaatttag

SEQ ID NO: 563 - DNA, 19 bp, Unknown
  Transcription factor binding site
aatttataacaccttttat

SEQ ID NO: 564 - DNA, 17 bp, Unknown
  Transcription factor binding site
ttttatgctaacgtttg

SEQ ID NO: 565 - DNA, 17 bp, Unknown
  Transcription factor binding site
gcaaacgttagcataaa

SEQ ID NO: 566 - DNA, 19 bp, Unknown
  Transcription factor binding site
gtttgccaacacttagcaa

SEQ ID NO: 567 - DNA, 17 bp, Unknown
  Transcription factor binding site
atttgcaagttgattaa

SEQ ID NO: 568 - DNA, 11 bp, Unknown
  Transcription factor binding site
tcaattaatca

SEQ ID NO: 569 - DNA, 15 bp, Unknown
  Transcription factor binding site
ttctaaattattttt

SEQ ID NO: 570 - DNA, 11 bp, Unknown
  Transcription factor binding site
taaattatttt

SEQ ID NO: 571 - DNA, 11 bp, Unknown
  Transcription factor binding site
aaaataattta

SEQ ID NO: 572 - DNA, 13 bp, Unknown
  Transcription factor binding site
atttttgtcttct

SEQ ID NO: 573 - DNA, 17 bp, Unknown
  Transcription factor binding site
gattagtatatgtattt

SEQ ID NO: 574 - DNA, 17 bp, Unknown
  Transcription factor binding site
tcaactggaaatgtaaa

SEQ ID NO: 575 - DNA, 17 bp, Unknown
  Transcription factor binding site
ggaaatgtaaatatttg

SEQ ID NO: 576 - DNA, 21 bp, Unknown
  Transcription factor binding site
attagcaaatatttacatttc

SEQ ID NO: 577 - DNA, 17 bp, Unknown
  Transcription factor binding site
tagtagaaatattagca

SEQ ID NO: 578 - DNA, 21 bp, Unknown
  Transcription factor binding site
attctcctatagtagaaatat

SEQ ID NO: 579 - DNA, 17 bp, Unknown
  Transcription factor binding site
ggagaattaaagtgagt

SEQ ID NO: 580 - DNA, 15 bp, Unknown
  Transcription factor binding site
aacaattaaatctcc

SEQ ID NO: 581 - DNA, 19 bp, Unknown
  Transcription factor binding site
gcattgcaacaattaaatc

SEQ ID NO: 582 - DNA, 27 bp, Unknown
  Transcription factor binding site
atgccatccatgcagcattgcaacaat

SEQ ID NO: 583 - DNA, 17 bp, Unknown
  Transcription factor binding site
tatgccatccatgcagc

SEQ ID NO: 584 - DNA, 17 bp, Unknown
  Transcription factor binding site
gtgtatatgccatccat

SEQ ID NO: 585 - DNA, 17 bp, Unknown
  Transcription factor binding site
tggcatatacaccaaac

SEQ ID NO: 586 - DNA, 11 bp, Unknown
  Transcription factor binding site
agaattattga

SEQ ID NO: 587 - DNA, 11 bp, Unknown
  Transcription factor binding site
tcaataattct

SEQ ID NO: 588 - DNA, 17 bp, Unknown
  Transcription factor binding site
attattatcctcaagaa

SEQ ID NO: 589 - DNA, 17 bp, Unknown
  Transcription factor binding site
ttgaggataataatggt

SEQ ID NO: 590 - DNA, 11 bp, Unknown
  Transcription factor binding site
accattattat

SEQ ID NO: 591 - DNA, 27 bp, Unknown
  Transcription factor binding site
gtgacgttcatgcacctcaaatcttgt

SEQ ID NO: 592 - DNA, 10 bp, Unknown
  Transcription factor binding site
natgcacctc

SEQ ID NO: 593 - DNA, 21 bp, Unknown
  Transcription factor binding site
tccacgtgacgttcatgcacc

SEQ ID NO: 594 - DNA, 21 bp, Unknown
  Transcription factor binding site
gtgcatgaacgtcacgtggac

SEQ ID NO: 595 - DNA, 10 bp, Unknown
  Transcription factor binding site
natgaacgtc

SEQ ID NO: 596 - DNA, 21 bp, Unknown
  Transcription factor binding site
ttttgtccacgtgacgttcat

SEQ ID NO: 597 - DNA, 21 bp, Unknown
  Transcription factor binding site
tgaacgtcacgtggacaaaag

SEQ ID NO: 598 - DNA, 17 bp, Unknown
  Transcription factor binding site
gaacgtcacgtggacaa

SEQ ID NO: 599 - DNA, 23 bp, Unknown
  Transcription factor binding site
aaccttttgtccacgtgacgttc

SEQ ID NO: 600 - DNA, 17 bp, Unknown
  Transcription factor binding site
ttgtccacgtgacgttc

SEQ ID NO: 601 - DNA, 17 bp, Unknown
  Transcription factor binding site
tttgtccacgtgacgtt

SEQ ID NO: 602 - DNA, 13 bp, Unknown
  Transcription factor binding site
gtcacgtggacaa

SEQ ID NO: 603 - DNA, 13 bp, Unknown
  Transcription factor binding site
ccttttgtccacg

SEQ ID NO: 604 - DNA, 17 bp, Unknown
  Transcription factor binding site
ggtttagtaatttttca

SEQ ID NO: 605 - DNA, 17 bp, Unknown
  Transcription factor binding site
tgtcttgaaaaattact

SEQ ID NO: 606 - DNA, 17 bp, Unknown
  Transcription factor binding site
tgtgtggtaacattgtt

SEQ ID NO: 607 - DNA, 17 bp, Unknown
  Transcription factor binding site
aacaatgttaccacaca

SEQ ID NO: 608 - DNA, 27 bp, Unknown
  Transcription factor binding site
catccatgcatgcacctcaaaacttgt

SEQ ID NO: 609 - DNA, 27 bp, Unknown
  Transcription factor binding site
ggggcatccatgcatgcacctcaaaac

SEQ ID NO: 610 - DNA, 27 bp, Unknown
  Transcription factor binding site
ttgaggtgcatgcatggatgcccctgt

SEQ ID NO: 611 - DNA, 10 bp, Unknown
  Transcription factor binding site
natgcacctc

SEQ ID NO: 612 - DNA, 17 bp, Unknown
  Transcription factor binding site
aggggcatccatgcatg

SEQ ID NO: 613 - DNA, 17 bp, Unknown
  Transcription factor binding site
aactttccacaggggca

SEQ ID NO: 614 - DNA, 17 bp, Unknown
  Transcription factor binding site
ccctgtggaaagtttaa

SEQ ID NO: 615 - DNA, 27 bp, Unknown
  Transcription factor binding site
atggcttccatgcaaatcatttccaaa

SEQ ID NO: 616 - DNA, 11 bp, Unknown
  Transcription factor binding site
gaaatgatttg

SEQ ID NO: 617 - DNA, 11 bp, Unknown
  Transcription factor binding site
caaatcatttc

SEQ ID NO: 618 - DNA, 11 bp, Unknown
  Transcription factor binding site
gaaatgatttg

SEQ ID NO: 619 - DNA, 17 bp, Unknown
  Transcription factor binding site
ttgcatggaagccatgt

SEQ ID NO: 620 - DNA, 17 bp, Unknown
  Transcription factor binding site
gttttacacatggcttc

SEQ ID NO: 621 - DNA, 13 bp, Unknown
  Transcription factor binding site
gccatgtgtaaaa

SEQ ID NO: 622 - DNA, 17 bp, Unknown
  Transcription factor binding site
tgtcatggttttacaca

SEQ ID NO: 623 - DNA, 15 bp, Unknown
  Transcription factor binding site
aataatgaagaaaac

SEQ ID NO: 624 - DNA, 17 bp, Unknown
  Transcription factor binding site
ttgcatgtaaatttgta

SEQ ID NO: 625 - DNA, 27 bp, Unknown
  Transcription factor binding site
caaatttacatgcaactagttatgcat

SEQ ID NO: 626 - DNA, 27 bp, Unknown
  Transcription factor binding site
atagactacatgcataactagttgcat

SEQ ID NO: 627 - DNA, 17 bp, Unknown
  Transcription factor binding site
tgcaactagttatgcat

SEQ ID NO: 628 - DNA, 17 bp, Unknown
  Transcription factor binding site
ctagttatgcatgtagt

SEQ ID NO: 629 - DNA, 27 bp, Unknown
  Transcription factor binding site
tagttatgcatgtagtctatataatga

SEQ ID NO: 630 - DNA, 17 bp, Unknown
  Transcription factor binding site
tagactacatgcataac

SEQ ID NO: 631 - DNA, 17 bp, Unknown
  Transcription factor binding site
atagactacatgcataa

(19) United States
(12) Patent Application Publication
(10) Pub. No.: US 2009/0217417 A1
(43) Pub. Date: Aug. 27, 2009

(54) Combination of lipid metabolism proteins and uses thereof

(75) Inventors: Thorsten Zank, Mannheim (DE); Oliver Oswald, Lautertal (DE)

Correspondence Address: CONNOLLY BOVE LODGE & HUTZ, LLP, P O BOX 2207, WILMINGTON, DE, 19899, US

(73) Assignee: BASF PLANT SCIENCE GMBH, LUDWIGSHAFEN (DE)

(21) Appl. No.: 11/989,249

(22) PCT Filed: Jul. 14, 2006
(86) PCT No.: PCT/EP2006/064276

§ 371 Date: Jan. 22, 2008

Related U.S. Application Data
(60) Provisional application No. 60/595,649, filed on Jul. 25, 2005.

(51) Int. Cl. A01H 1/00; C07H 21/00; C07K 2/00; C12N 15/63; A01H 5/00; A01H 5/10

(52) U.S. Cl. 800/281; 536/23.1; 530/300; 536/23.6; 536/23.7; 435/320.1; 800/278; 800/298; 800/312; 800/320.1; 800/320; 800/320.3; 800/320.2; 800/317.1; 800/314

(57) ABSTRACT

Described herein are inventions in the field of genetic engineering of plants, including combinations of nucleic acid molecules encoding LMPs to improve agronomic, horticultural, and quality traits. This invention relates generally to the combination of nucleic acid sequences encoding proteins that are related to the presence of seed storage compounds in plants. More specifically, the present invention relates to LMP nucleic acid sequences encoding lipid metabolism proteins (LMP) and the use of these combinations of these sequences, their order and direction in the combination, and the regulatory elements used to control expression and transcript termination in these combinations in transgenic plants. In particular, the invention is directed to methods for manipulating fatty acid-related compounds and for increasing oil level and altering the fatty acid composition in plants and seeds. The invention further relates to methods of using these novel combinations of polypeptides to stimulate plant growth and/or to increase yield and/or composition of seed storage compounds.

### FIELD OF THE INVENTION

Described herein are inventions in the field of genetic engineering of plants, including combinations of nucleic acid molecules encoding LMPs to improve agronomic, horticultural, and quality traits. This invention relates generally to the combination of nucleic acid sequences encoding proteins that are related to the presence of seed storage compounds in plants. More specifically, the present invention relates to LMP nucleic acid sequences encoding lipid metabolism proteins (LMP) and the use of these combinations of these sequences, their order and direction in the combination, and the regulatory elements used to control expression and transcript termination in these combinations in transgenic plants. In particular, the invention is directed to methods for manipulating fatty acid-related compounds and for increasing oil level and altering the fatty acid composition in plants and seeds. The invention further relates to methods of using these novel combinations of polypeptides to stimulate plant growth and/or to increase yield and/or composition of seed storage compounds.

### BACKGROUND OF THE INVENTION

The study and genetic manipulation of plants has a long history that began even before the famed studies of Gregor Mendel. In perfecting this science, scientists have accomplished modification of particular traits in plants ranging from potato tubers having increased starch content to oilseed plants such as canola and sunflower having increased or altered fatty acid content. With the increased consumption and use of plant oils, the modification of seed oil content and seed oil levels has become increasingly widespread (e.g. Töpfer et al. 1995, Science 268:681-686). Manipulation of biosynthetic pathways in transgenic plants provides a number of opportunities for molecular biologists and plant biochemists to affect plant metabolism giving rise to the production of specific higher-value products. The seed oil production or composition has been altered in numerous traditional oilseed plants such as soybean (U.S. Pat. No. 5,955,650), canola (U.S. Pat. No. 5,955,650), sunflower (U.S. Pat. No. 6,084,164), and rapeseed (Töpfer et al. 1995, Science 268:681-686), and non-traditional oil seed plants such as tobacco (Cahoon et al. 1992, Proc. Natl. Acad. Sci. USA 89:11184-11188).

Plant seed oils comprise both neutral and polar lipids (see Table 2). The neutral lipids contain primarily triacylglycerol, which is the main storage lipid that accumulates in oil bodies in seeds. The polar lipids are mainly found in the various membranes of the seed cells, e.g. the endoplasmic reticulum, microsomal membranes, plastidial and mitochondrial membranes and the cell membrane. The neutral and polar lipids contain several common fatty acids (see Table 3) and a range of less common fatty acids. The fatty acid composition of membrane lipids is highly regulated and only a select number of fatty acids are found in membrane lipids. On the other hand, a large number of unusual fatty acids can be incorporated into the neutral storage lipids in seeds of many plant species (Van de Loo F. J. et al. 1993, Unusual Fatty Acids in Lipid Metabolism in Plants pp. 91-126, editor T S Moore Jr. CRC Press; Millar et al. 2000, Trends Plant Sci. 5:95-101).

Lipids are synthesized from fatty acids and their synthesis may be divided into two parts: the prokaryotic pathway and the eukaryotic pathway (Browse et al. 1986, Biochemical J. 235:25-31; Ohlrogge & Browse 1995, Plant Cell 7:957-970). The prokaryotic pathway is located in plastids that are also the primary site of fatty acid biosynthesis. Fatty acid synthesis begins with the conversion of acetyl-CoA to malonyl-CoA by acetyl-CoA carboxylase (ACCase). Malonyl-CoA is converted to malonyl-acyl carrier protein (ACP) by the malonyl-CoA:ACP transacylase. The enzyme beta-keto-acyl-ACP-synthase III (KAS III) catalyzes a condensation reaction, in which the acyl group from acetyl-CoA is transferred to malonyl-ACP to form 3-ketobutyryl-ACP. In a subsequent series of condensation, reduction and dehydration reactions the nascent fatty acid chain on the ACP cofactor is elongated by the step-by-step addition (condensation) of two carbon atoms donated by malonyl-ACP until a 16- or 18-carbon saturated fatty acid chain is formed. The plastidial delta-9 acyl-ACP desaturase introduces the first double bond into the fatty acid.

In the prokaryotic pathway the saturated and monounsaturated acyl-ACPs are direct substrates for the plastidial glycerol-3-phosphate acyltransferase and the lysophosphatidic acid acyltransferase, which catalyze the esterification of glycerol-3-phosphate at the sn-1 and sn-2 position. The resulting phosphatidic acid is the precursor for plastidial lipids, in which further desaturation of the acyl-residues can occur.

In the eukaryotic lipid biosynthesis pathway thioesterases cleave the fatty acids from the ACP cofactor and free fatty acids are exported to the cytoplasm where they participate as fatty acyl-CoA esters in the eukaryotic pathway. In this pathway the fatty acids are esterified by glycerol-3-phosphate acyltransferase and lysophosphatidic acid acyl-transferase to the sn-1 and sn-2 positions of glycerol-3-phosphate, respectively, to yield phosphatidic acid (PA). The PA is the precursor for other polar and neutral lipids, the latter being formed in the Kennedy ot other pathways (Voelker 1996, Genetic Engineering ed.: Setlow 18:111-113; Shanklin & Cahoon 1998, Annu. Rev. Plant Physiol. Plant Mol. Biol. 49:611-641; Frentzen 1998, Lipids 100:161-166; Millar et al. 2000, Trends Plant Sci. 5:95-101).

The acyl-CoAs resulted from the export of plastidic fatty acids can also be elongated to yield very-long-chain fatty acids with more than 18 carbon atoms. Fatty acid elongases are multienzyme complexes consisting of at least four enzyme activities: beta-ketoacyl-CoA synthases, beta-ketoacyl-CoA reductase, beta-hydroxyacyl-CoA dehydratase and enoyl-CoA reductase. It is well known that the beta-ketoacyl-CoA synthase determines the activity and the substrate selectivity of the fatty acid elongase complex (Millar & Kunst 1997, Plant J. 12:121-131). The very-long-chain fatty acids can be either used for wax and sphingolipid biosynthesis or enter the pathways for seed storage lipid biosynthesis.

Storage lipids in seeds are synthesized from carbohydrate-derived precursors. Plants have a complete glycolytic pathway in the cytosol (Plaxton 1996, Annu. Rev. Plant Physiol. Plant Mol. Biol. 47:185-214), and it has been shown that a complete pathway also exists in the plastids of rapeseeds (Kang & Rawsthorne 1994, Plant J. 6:795-805). Sucrose is the primary source of carbon and energy, transported from the leaves into the developing seeds. During the storage phase of seeds, sucrose is converted in the cytosol to provide the metabolic precursors glucose-6-phosphate and pyruvate. These are transported into the plastids and converted into acetyl-CoA that serves as the primary precursor for the synthesis of fatty acids. Acetyl-CoA in the plastids is the central precursor for lipid biosynthesis. Acetyl-CoA can be formed in the plastids by different reactions and the exact contribution of each reaction is still being debated (Ohlrogge & Browse 1995, Plant Cell 7:957-970). It is however accepted that a large part of the acetyl-CoA is derived from glucose-6-phospate and pyruvate that are imported from the cytoplasm into the plastids. Sucrose is produced in the source organs (leaves, or anywhere where photosynthesis occurs) and is transported to the developing seeds that are also termed sink organs. In the developing seeds, sucrose is the precursor for all the storage compounds, i.e. starch, lipids, and partly the seed storage proteins.

Generally the breakdown of lipids is considered to be performed in plants in peroxisomes in the process know as beta-oxidation. This process involves the enzymatic reactions of acyl-CoA oxidase, hydroxyacyl-CoA-dehydrogenase (both found as a multifunctional complex) and ketoacyl-CoA-thiolase, with catalase in a supporting role (Graham and Eastmond 2002). In addition to the breakdown of common fatty acids beta-oxidation also plays a role in the removal of unusual fatty acids and fatty acid oxidation products, the glyoxylate cycle and the metabolism of branched chain amino acids (Graham and Eastmond 2002).

Storage compounds, such as triacylglycerols (seed oil), serve as carbon and energy reserves, which are used during germination and growth of the young seedling. Seed (vegetable) oil is also an essential component of the human diet and a valuable commodity providing feedstocks for the chemical industry.

Although the lipid and fatty acid content, and/or composition of seed oil, can be modified by the traditional methods of plant breeding, the advent of recombinant DNA technology has allowed for easier manipulation of the seed oil content of a plant, and in some cases, has allowed for the alteration of seed oils in ways that could not be accomplished by breeding alone (see, e.g., Töpfer et al., 1995, Science 268:681-686). For example, introduction of a Δ12-hydroxylase nucleic acid sequence into transgenic tobacco resulted in the introduction of a novel fatty acid, ricinoleic acid, into the tobacco seed oil (Van de Loo et al. 1995, Proc. Natl. Acad. Sci USA 92:6743-6747). Tobacco plants have also been engineered to produce low levels of petroselinic acid by the introduction and expression of an acyl-ACP desaturase from coriander (Cahoon et al. 1992, Proc. Natl. Acad. Sci USA 89:11184-11188).

The modification of seed oil content in plants has significant medical, nutritional and economic ramifications. With regard to the medical ramifications, the long chain fatty acids (C18 and longer) found in many seed oils have been linked to reductions in hypercholesterolemia and other clinical disorders related to coronary heart disease (Brenner 1976, Adv. Exp. Med. Biol. 83:85-101). Therefore, consumption of a plant having increased levels of these types of fatty acids may reduce the risk of heart disease. Enhanced levels of seed oil content also increase large-scale production of seed oils and thereby reduce the cost of these oils.

In order to increase or alter the levels of compounds such as seed oils in plants, nucleic acid sequences and proteins regulating lipid and fatty acid metabolism must be identified. As mentioned earlier, several desaturase nucleic acids such as the Δ6-desaturase nucleic acid, Δ12-desaturase nucleic acid and acyl-ACP desaturase nucleic acid have been cloned and demonstrated to encode enzymes required for fatty acid synthesis in various plant species. Oleosin nucleic acid sequences from such different species as canola, soybean, carrot, pine, and Arabidopsis thaliana have also been cloned and determined to encode proteins associated with the phospholipid monolayer membrane of oil bodies in those plants.

It has also been determined that two phytohormones, gibberellic acid (GA) and absisic acid (ABA), are involved in overall regulatory processes in seed development (e.g. Ritchie & Gilroy, 1998, Plant Physiol. 116:765-776; Arenas-Huertero et al., 2000, Genes Dev. 14:2085-2096). Both the GA and ABA pathways are affected by okadaic acid, a protein phosphatase inhibitor (Kuo et al. 1996, Plant Cell. 8:259-269). The regulation of protein phosphorylation by kinases and phosphatases is accepted as a universal mechanism of cellular control (Cohen, 1992, Trends Biochem. Sci. 17:408-413). Likewise, the plant hormones ethylene (e.g. Zhou et al., 1998, Proc. Natl. Acad. Sci. USA 95:10294-10299; Beaudoin et al., 2000, Plant Cell 2000:1103-1115) and auxin (e.g. Colon-Carmona et al., 2000, Plant Physiol. 124:1728-1738) are involved in controlling plant development as well.

Although several compounds are known that generally affect plant and seed development, there is a clear need to specifically identify factors, and particularly combinations thereof, that are more specific for the developmental regulation of storage compound accumulation and to identify combination of genes which have the capacity to confer altered or increased oil production to its host plant and to other plant species. This invention discloses combinations of nucleic acid sequences from Physcomitrella patens and Arabidopsis thaliana. These combinations of nucleic acid sequences can be used to alter or increase the levels of seed storage compounds such as proteins, sugars and oils, in plants, including transgenic plants, such as canola, linseed, soybean, sunflower, maize, oat, rye, barley, wheat, rice, pepper, tagetes, cotton, oil palm, coconut palm, flax, castor, and peanut, which are oilseed plants containing high amounts of lipid compounds.

### SUMMARY OF THE INVENTION

The present invention provides novel combinations isolated nucleic acid coding for LMPs and order thereof within the combinations, resulting in coordinated presence of proteins associated with the metabolism of seed storage compounds in plants

Also provided by the present invention are regulatory genetic elements such as promoters and terminators particularly suited for the expression of combinations of more than one LMPs.

Also provided in the present invention is an arrangement of regulatory elements and genes encoding for LMPs to enhance their effect on seed storage compounds.

A further object of the present invention is an isolated nucleic acid comprising two or more LMP polynucleotide sequences selected from the group consisting of:
a. a polynucleotide sequence as described by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15;
b. a polynucleotide sequence encoding a polypeptide as described by SEQ ID NO: 2, SEQ ID NO: 4, SEQ ID NO: 6 or SEQ ID NO: 8, SEQ ID NO: 10, SEQ ID NO: 12, SEQ ID NO: 14 or SEQ ID NO: 16;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

Preferably, the isolated nucleic acid of the present invention encodes a polypeptide that functions as a modulator of a seed storage compound in microorganisms or plants. The nucleic acid of the present invention can comprise one, two, three, four, five, six, seven or eight of the nucleotide sequences of the present invention, preferably of the nucleotide sequences as described by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15. Especially preferred LMP nucleic acid sequences are shown in the following table, wherein the sequence identifier are those used in WIPO Standard ST. 25 sequence listing.

TABLE 1
Combination of LMP nucleotide sequences

| | Nucleotide | Nucleotide | Nucleotide |
|---|---|---|---|
| Combination of | sequence as | sequence as | sequence as |
| nucleotide | described by | described by | described by |
| sequences | SEQ ID NO: | SEQ ID NO: | SEQ ID NO: |
| 21 | 1 | 11 | |
| 22 | 1 | 9 | |
| 23 | 7 | 11 | |
| 24 | 1 | 15 | |
| 25 | 1 | 3 | |
| 26 | 3 | 7 | |
| 27 | 3 | 9 | |
| 28 | 5 | 13 | |
| 29 | 1 | 9 | 13 |
| 30 | 1 | 7 | 3 |
| 31 | 9 | 15 | 13 |
| 32 | 5 | 3 | |
| 33 | 1 | 5 | 13 |
| 34 | 3 | 15 | 13 |
| 35 | 9 | 5 | 13 |
| 36 | 9 | 7 | 13 |
| 37 | 7 | 9 | 13 |
| 38 | 7 | 3 | 13 |
| 39 | 7 | 11 | 13 |
| 40 | 9 | 11 | 13 |
| 41 | 3 | 11 | 13 |
| 42 | 3 | 5 | |

Especially preferred are combinations number 21, 23, 26, 27, 32 & 33 of table 1. Further preferred nucleic acid sequences are the combinations of polynucleotide sequences shown in FIG. 8, Table 9 Table 9. Especially preferred are combinations number 21, 23, 26, 27, 32 & 33 of FIG. 8, Table 9 Table 9. The nucleic acids of the present invention, particularly those combinations of polynucleotide sequences shown in FIG. 8, Table 9 Table 9, further preferred combinations number 21, 23, 26, 27, 32 & 33 of FIG. 8, Table 9 Table 9 can be used to increase the seed oil content in seeds, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Preferably the modification of the level or composition of a seed storage compound is measured as dry weight as measured by gas chromatography in the seed.

A further object is an isolated polypeptide encoded by a polynucleotide sequence above. Preferably the isolated polypeptide sequence of the present invention functions as a modulator of a seed storage compound in microorganisms or plants.

A further object of the present invention is an expression vector containing the nucleic acid of the present invention, wherein the nucleic acid is operatively linked to a promoter selected from the group consisting of a seed-specific promoter, a root-specific promoter, a leaf specific promoter and a non-tissue-specific promoter. Preferably the expression vector contains the combinations of polynucleotide sequences shown in FIG. 8, Table 9. Especially preferred are combinations number 21, 23, 26, 27, 32 & 33 of FIG. 8, Table 9.

By promoter is meant a polynucleotide sequence upstream from the transcriptional initiation site and which contains the regulatory regions required for transcription.

Preferably the promoter of the present invention is selected from the group consisting of:
a. a polynucleotide sequence as described by SEQ ID NO: 9, SEQ ID NO: 10, SEQ ID NO: 11, SEQ ID NO: 12 or SEQ ID NO: 13;
b. a polynucleotide sequence having at least 70% sequence identity with the nucleic acid of a) above;
c. a polynucleotide sequence that hybridizes under stringent conditions to nucleic acid of a) or b) above;
d. a polynucleotide sequence comprising at least 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences as described by the sequence identifiers (SEQ ID NO:) in the sequence listing of the present application of the polynucleotide sequences as described by the capital letters (e.g. ACAC for the polynucleotide sequence as described by SEQ ID NO: 26) of the polynucleotide sequence as described by SEQ ID NO: 26-156 related to a promoter as described by columns 1 and 2 of table 10 (e.g. for the promoter as described by SEQ ID NO: 9 at least 50% of the polynucleotide sequences as described by SEQ ID NO: 44, 45, 46, 46, 48, 54, 59, 59, 59, 62, 63, 68, 70, 80, 80, 80, 81, 84, 84, 85, 87, 96, 97, 100, 105, 106, 108, 108, 108, 109, 114, 115, 115, 124, 124, 125, 135, 135, 136, 136, 141, 141, 142, 142, 142, 142, 144, 146, 146, 146, 148, 149, 152, 154, 154, 154. That means for the promoter as described by SEQ ID NO: 9 at least 50% of the polynucleotide sequences as described by column 2, lines 2 to 57, preferably for the promoter as described by SEQ ID NO: 10 at least 50% of the polynucleotide sequences as described by column 2, lines 58 to 134. A polynucleotide sequence as described by SEQ ID NO: 26-156 can occur one or more times, e.g. 1, 2, 3, 4, 5, 6, 7, 8, 9, 10 or more times in the promoter of the present invention, preferably as shown by the repetitions of polynucleotide sequences as described by the sequence identifiers in column 2 of table 10 of the promoter sequences as described by SEQ ID NO: 9-13); and
e. a polynucleotide sequence comprising a polynucleotide sequence having at least 70%, preferably 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the full length polynucleotide sequence as described by the capital letters of the polynucleotide sequence as described by SEQ ID NO: 26-156, wherein the polynucleotide sequence comprises 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences of the nucleotide sequences having at least 70% preferably 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the polynucleotide sequence as described by the capital letters of the polynucleotide sequence as described by SEQ ID NO: 26-156 related to a promoter as described by columns 1 and 2 of table 10. The percent sequence identity between two polynucleotide sequences that are comprised in a promoter of the present invention is determined as the so called Core Similarity using the function MatInspector with default parameters of GEMS Launcher 4.2.2 software package (Genomatix Software GmbH). The algorithm underlying the Core Similarity is disclosed on pages 4879-4880 of Quandt K, Frech K, Karas H, Wingender E, Werner T (1995), Matind and MatInspector: new fast and versatile tools for detection of consensus matches in nucleotide sequence data. Nucleic Acids Res. 23, 4878-4884, [PUBMED: 96128303])

In a preferred embodiment the promoter of the present invention comprises at least 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences of the full-length polynucleotide sequences as described by SEQ ID NO: 26-156 related to a promoter as described by columns 1 and 2 of table 10.

In a further preferred embodiment the promoter of the present invention comprises a polynucleotide sequence having at least 70%, preferably 75%, 80%, 81%, 82%, 83%, 84%, 85%, 86%, 87%, 88%, 89%, 90%, 91%, 92%, 93%, 94%, 95%, 96%, 97%, 98%, 99% sequence identity with the full length polynucleotide sequence as described by SEQ ID NO: 26-156, wherein the polynucleotide sequence comprises 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences as described by the sequence identifiers (SEQ ID NO:) related to a promoter as described by columns 1 and 2 of table 10 in the sequence listing of the present application of the nucleotide sequences having at least 70% sequence identity with the polynucleotide sequence as described by SEQ ID NO: 26-156. The percent sequence identity between two polynucleotide sequences that are comprised in a promoter of the present invention is determined as the so called Matrix Similarity using the function MatInspector with default parameters of GEMS Launcher 4.2.2 software package (Genomatix Software GmbH). The algorithm underlying the Matrix Similarity is disclosed on pages 4879-4880 of Quandt K, Frech K, Karas H, Wingender E, Werner T (1995), MatInd and MatInspector: new fast and versatile tools for detection of consensus matches in nucleotide sequence data. Nucleic Acids Res. 23, 4878-4884, [PUBMED: 96128303].

In a further preferred embodiment the promoter of the present invention comprises at least 50%, preferably 60%, 70%, 80%, 81%, 82%, 83%, 84%, 85%, 86%, 87%, 88%, 89%, 90%, 91%, 92%, 93%, 94%, 95%, 96%, 97%, 98% or 99% by number of polynucleotide sequences of the polynucleotide sequences as described by the capital letters of the polynucleotide sequence as described by SEQ ID NO: 157-631 related to a promoter as described by columns 1 and 3 of table 10. For example for the promoter as described by SEQ ID NO: 9 at least 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences as described by the sequence identifiers (SEQ ID NO:) in the sequence listing of the present application of the polynucleotide sequences as described by SEQ ID NO: 263, 285, 303, 304, 313, 301, 260, 268, 271, 265, 264, 279, 277, 282, 294, 305, 290, 308, 310, 270, 278, 281, 262, 289, 300, 292, 275, 283, 287, 296, 293, 280, 286, 261, 314, 298, 272, 291, 307, 312, 297, 311, 276, 295, 302, 306, 267, 269, 274, 309 are comprised.

In a further preferred embodiment the promoter of the present invention comprises a polynucleotide sequence having at least 70%, preferably 75%, 80%, 81%, 82%, 83%, 84%, 85%, 86%, 87%, 88%, 89%, 90%, 91%, 92%, 93%, 94%, 95%, 96%, 97%, 98%, 99% sequence identity with the polynucleotide sequences as described by the capital letters of the polynucleotide sequence as described by SEQ ID NO: 157-631, wherein the polynucleotide sequence comprises 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of the polynucleotide sequences related to a promoter as described by columns 1 and 3 of table 10 having at least 70% sequence identity with the polynucleotide sequence as described by the capital letters of the polynucleotide sequence as described by SEQ ID NO: 157-631.

In a further preferred embodiment the promoter comprises at least 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of polynucleotide sequences of the full-length polynucleotide sequences as described by SEQ ID NO: 157-631 related to a promoter as described by columns 1 and 3 of table 10.

In a further preferred embodiment the promoter comprises a polynucleotide sequence having at least 70%, preferably 75%, 80%, 81%, 82%, 83%, 84%, 85%, 86%, 87%, 88%, 89%, 90%, 91%, 92%, 93%, 94%, 95%, 96%, 97%, 98%, 99% sequence identity with the polynucleotide sequences as described by SEQ ID NO: 157-631, wherein the polynucleotide sequence comprises 50%, preferably 60%, 70%, 80%, 81%, 82%, 83%, 84%, 85%, 86%, 87%, 88%, 89%, 90%, 91%, 92%, 93%, 94%, 95%, 96%, 97%, 98%, 99% by number of the polynucleotide sequences related to a promoter as described by columns 1 and 3 of table 10 having at least 70% preferably 75%, 80%, 81%, 82%, 83%, 84%, 85%, 86%, 87%, 88%, 89%, 90%, 91%, 92%, 93%, 94%, 95%, 96%, 97%, 98%, 99% sequence identity with the polynucleotide sequence as described by SEQ ID NO: 157-631,

Preferably the expression vector of the present invention contains a terminator selected from the group consisting of:
a. a polynucleotide sequence as described by SEQ ID NO: 22, SEQ ID NO: 23, SEQ ID NO: 24 or SEQ ID NO: 25;
b. a polynucleotide sequence having at least 70% sequence identity with the nucleic acid of a) above;
c. a polynucleotide sequence that is complementary to the nucleic acid of a) or b) above; and
d. a polynucleotide sequence that hybridizes under stringent conditions to nucleic acid of a) or b) above.

A further object of the present invention is a method of producing a transgenic plant having a modified level of a seed storage compound weight percentage compared to an empty vector control comprising
a. a first step of introduction into a plant cell of an expression vector containing a nucleic acid, and
b. a further step of generating from the plant cell the transgenic plant,
   wherein the nucleic acid encodes a polypeptide that functions as a modulator of a seed storage compound in the plant, and wherein the nucleic acid comprises a polynucleotide sequence of the present invention. In a preferred embodiment of the method of the present invention the nucleic acid comprises a polynucleotide sequence having at least 90% sequence identity with the polynucleotide sequence of the present invention, preferably the combinations of polynucleotide sequences shown in FIG. 8, Table 9. Especially preferred are combinations number 21, 23, 26, 27, 32 & 33 of FIG. 8, Table 9.

Preferably the total seed oil content weight percentage is increased in the transgenic plant as compared to an empty vector control, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Preferably for the purposes of this invention the modification of the level or composition of a seed storage compound is measured as dry weight as measured by gas chromatography in the seed.

The percent increases of a seed storage compound are generally determined compared to an empty vector control. An empty vector control is a transgenic plant, which has been transformed with the same vector or construct as a transgenic plant according to the present invention except for such a vector or construct lacking the nucleic acid sequences of the present inventions, preferably the nucleic acid sequences as disclosed in Appendix A. An empty vector control is shown for example in example 9.

A further object of the present invention is a method of modulating the level of a seed storage compound weight percentage in a plant comprising, modifying the expression of a nucleic acid in the plant, comprising
a. a first step of introduction into a plant cell of an expression vector comprising a nucleic acid, and
b. a further step of generating from the plant cell the transgenic plant,
   wherein the nucleic acid encodes a polypeptide that functions as a modulator of a seed storage compound in the plant wherein the nucleic acid comprises the polynucleotide sequence of the present invention, preferably the combinations of polynucleotide sequences shown in FIG. 8, Table 9. Especially preferred are combinations number 21, 23, 26, 27, 32 & 33 of FIG. 8, Table 9.

The method of Claim 11, wherein the total seed oil content weight percentage is increased in the transgenic plant as compared to an empty vector control, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

A further object of the present invention is a transgenic plant made by the method of the present invention. The transgenic plant of Claim 13, wherein the total seed oil content weight percentage is increased in the transgenic plant as compared to an empty vector control, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Preferably the transgenic plant of the present invention is selected from the group consisting of rapeseed, canola, linseed, soybean, sunflower, maize, oat, rye, barley, wheat, rice, pepper, tagetes, cotton, oil palm, coconut palm, flax, castor, sugarbeet, rice and peanut.

A further object of the present invention is a seed produced by the transgenic plant of the present invention, wherein the plant expresses the polypeptide that functions as a modulator of a seed storage compound and wherein the plant is true breeding for a modified level of seed storage compound weight percentage as compared to an empty vector control. The modification can be an increase or a decrease, preferably an increase of the seed storage compound, preferably of the seed oil content by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Preferably the modification of the level or composition of a seed storage compound is measured as dry weight as measured by gas chromatography in the seed.

Additionally, the present invention relates to and provides the use of combinations LMP nucleic acids in the production of transgenic plants having a modified level or composition of a seed storage compound, preferably of seed oil, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

For the purposes of the present invention, preferably the modification of the level or composition of a seed storage compound is measured as dry weight by gas chromatography in the seed.

In regard to an altered composition, the present invention can be used to, for example, increase the percentage of oleic acid relative to other plant oils, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. A method of producing a transgenic plant with a modified level or composition of a seed storage compound includes the steps of transforming a plant cell with an expression vector comprising an LMP nucleic acid, and generating a plant with a modified level or composition of the seed storage compound from the plant cell. In a preferred embodiment, the plant is an oil producing species selected from the group consisting of canola, linseed, soybean, sunflower, maize, oat, rye, barley, wheat, rice, pepper, tagetes, cotton, oil palm, coconut palm, flax, castor, and peanut, for example.

According to the present invention, the compositions and methods described herein can be used to alter the composition of more than one LMP in a transgenic plant and to increase or decrease the level of more than one LMP in a transgenic plant comprising increasing or decreasing the expression of more than one LMP nucleic acid in the plant, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Increased or decreased expression of the LMP nucleic acid can be achieved through transgenic overexpression, cosuppression approaches, antisense approaches, and in vivo mutagenesis of the LMP nucleic acid. The present invention can also be used to increase or decrease the level of a lipid in a seed oil, to increase or decrease the level of a fatty acid in a seed oil, or to increase or decrease the level of a starch in a seed or plant, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

More specifically, the present invention includes and provides a method for increasing total oil content in a seeds, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control comprising: transforming a plant with a nucleic acid construct that comprises as operably linked components, combinations of two or more promoters and nucleic acid sequences encoding for LMPs, and growing the plant. Furthermore, the present invention includes and provides a method for increasing the level of oleic acid in a seed, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control comprising: transforming a plant with a nucleic acid construct that comprises as operably linked components, combinations of two or more promoters and nucleic acid sequences capable of increasing the level of oleic acid, and growing the plant.

Also included herein is a seed produced by a transgenic plant transformed by a combination of LMP DNA sequences, wherein the seed contains the LMP DNA sequences in a combination as described within this invention and wherein the plant is true breeding for a modified level of a seed storage compound. The present invention additionally includes a seed oil produced by the aforementioned seed.

Further provided by the present invention are vectors comprising the nucleic acids and combinations of the later, host cells containing the vectors, and descendent plant materials produced by transforming a plant cell with the nucleic acids and/or vectors.

According to the present invention, the compounds, compositions, and methods described herein can be used to increase or decrease the relative percentages of a lipid in a seed oil, increase or decrease the level of a lipid in a seed oil, or to increase or decrease the level of a fatty acid in a seed oil, or to increase or decrease the level of a starch or other carbohydrate in a seed or plant, or to increase or decrease the level of proteins in a seed or plant. The manipulations described herein can also be used to improve seed germination and growth of the young seedlings and plants and to enhance plant yield of seed storage compounds, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

It is further provided a method of producing a higher or lower, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control, than normal or typical level of storage compound in a transgenic plant expressing a combination of LMP nucleic acids in the transgenic plant, wherein the transgenic plant is Arabidopsis thaliana, Brassica napus, Glycine max, Oryza sativa, Zea mays, Triticum aestivum, Helianthus anuus or Beta vulgaris or a species different from Arabidopsis thaliana, Brassica napus, Glycine max, Oryza sativa or Triticum aestivum. Also included herein are compositions and methods of the modification of the efficiency of production of a seed storage compound. As used herein, where the phrase Arabidopsis thaliana, Brassica napus, Glycine max, Oryza sativa, Zea mays, Triticum aestivum, Helianthus anuus or Beta vulgaris is used, this also means Arabidopsis thaliana and/or Brassica napus and/or Glycine max and/or Oryza sativa and/or Triticum aestivum and/or Zea mays and/or Helianthus anuus and/or Beta vulgaris.

Accordingly, it is an object of the present invention to provide novel combinations of LMP nucleic acids and resulting in coordinate production of LMP amino acid sequences, as well as active fragments, analogs, and orthologs thereof. Those active fragments, analogs, and orthologs can also be from different plant species as one skilled in the art will appreciate that other plant species will also contain those or related nucleic acids.

It is another object of the present invention to provide transgenic plants having modified levels of seed storage compounds, and in particular, modified levels of a lipid, a fatty acid, or a sugar, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

The polynucleotides and combinations of the later of the present invention, including agonists and/or fragments thereof and of their encoded amino acid sequences, have also uses that include modulating plant growth, and potentially plant yield, preferably increasing plant growth under adverse conditions (drought, cold, light, UV). In addition, antagonists of the present invention may have uses that include modulating plant growth and/or yield, through preferably increasing plant growth and yield. In yet another embodiment, over-expression polypeptides of the present invention using a constitutive promoter may be useful for increasing plant yield under stress conditions (drought, light, cold, UV) by modulating light utilization efficiency. Moreover, polynucleotides and polypeptides of the present invention will improve seed germination and seed dormancy and, hence, will improve plant growth and/or yield of seed storage compounds.

The combination of nucleic acid molecules of the present invention may further comprise combinations of operably linked promoter or partial promoter region. The promoters can be a constitutive promoter, an inducible promoter or a tissue-specific promoter. The constitutive promoter can be, for example, the superpromoter (Ni et al., Plant J. 7:661-676, 1995; U.S. Pat. No. 5,955,646) or the PtxA promoter (PF 55368-2 US, Song H. et al., 2004, see Example 11). The tissue-specific promoter can be active in vegetative tissue or reproductive tissue. The tissue-specific promoter active in reproductive tissue can be a seed-specific promoter. The seed-specific promoter can be, for example, the USP promoter (Bäumlein et al. 1991, Mol. Gen. Genetics 225:459-67) or the leguminB4 promoter (Bäumlein et al. 1992, Plant Journal 2(2): 233-238). The tissue-specific promoter active in vegetative tissue can be a root-specific, shoot-specific, meristem-specific or leaf-specific promoter. The isolated nucleic acid molecule of the present invention can still further comprise a 5′ non-translated sequence, 3′ non-translated sequence, introns, or the combination thereof.

The present invention also provides a method for increasing the number and/or size of one or more plant organs of a plant expressing a combination of nucleic acids encoding Lipid Metabolism Proteins (LMP), or a portion thereof, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. More specifically, seed size and/or seed number and/or weight might be manipulated. Moreover, root length can be increased, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control. Longer roots can alleviate not only the effects of water depletion from soil but also improve plant anchorage/standability thus reducing lodging. Also, longer roots have the ability to cover a larger volume of soil and improve nutrient uptake. All of these advantages of altered root architecture have the potential to increase crop yield. Additionally, the number and size of leaves might be increased by the nucleic acid sequences provided in this application. This will have the advantage of improving photosynthetic light utilization efficiency by increasing photosynthetic lightcapture capacity and photosynthetic efficiency, by e.g. 1, 2.5, 5, 7.5, 10, 12.5, 15, 17.5, 20, 22.5, 25, 27.5 or 30% by weight or more, preferably by 5% by weight or more, more preferably by 7.5% by weight or more and even more preferably by 10% by weight or more as compared to an empty vector control.

It is a further object of the present invention to provide methods for producing such aforementioned transgenic plants.

It is another object of the present invention to provide seeds and seed oils from such aforementioned transgenic plants.

These and other objects, features, and advantages of the present invention will become apparent after a review of the following detailed description of the disclosed embodiments and the appended claims.

### BRIEF DESCRIPTION OF THE DRAWINGS

FIGS. 1A-H. SEQ ID NO:1-8—open reading frame of the nucleic acid sequence coding for LMP useful in novel combinations to increase seed storage compounds.

FIGS. 2A-E. SEQ ID NO:9-13 Nucleic acid sequences of exemplary promoters useful in novel combinations to increase seed storage compounds.

FIGS. 3A-3B. SEQ ID NO:5-614-17—Nucleic acid sequences of exemplary terminators useful in novel combinations to increase seed storage compounds.

FIG. 4: Schematic representation of the binary vector pSUN indicating relevant features and restriction sites. b-RB=right border of T-DNA; c-aadA=aminoglycoside 3′-adenylyl-transferase codons; o-ColE1 replication origin of the plasmid pBR322, consisting of the two components o-REP-ColE1 and o-BOM-ColE1; VS1-rep=replication origin and repA of plasmid pVS1 VS1-sta=sta gene from plasmid pVS1; b-LB=left border of T-DNA; T-DNA cassette marks the region where the different T-DNA cassette for the different constructs are located. These T-DNA cassettes are described in FIG. 5.

FIG. 5: Schematic representation of the T-DNA cassette containing the arrangement of the novel combination of genes coding for LMPs. Positions within the combination are given by the letters A-C, SM denotes the selection marker cassette elements (promoter, selection marker gene & terminator); b-LB=left border of T-DNA, b-RB=right border of T-DNA

FIG. 6: Graphical representation of the seed oil content of Arabidopsis T2 seeds carrying combinations of LMPs number 21, 23, 26, 27, 32 & 33 (see table 9 of FIG. 8). Graphs represent the g fatty acids in the seed per g dry weight as measured by gas chromatography. Black bars represent lines carrying the combinations, empty bars represent the values from 3 empty vector controls. Each value is the mean of at least duplicate extractions and measurements, error bars represent the standard deviation. The control value given is the mean of 3 to 8 empty vector controls extracted and measured in at least duplicate. Table 7 of FIG. 6 provides the peak relative increase in seed oil content of T2 Arabidopsis seed harboring the combinations of LMPS as measured by gas chromatography as described above.

As a further example the data shown in table 8 in FIG. 7 demonstrates that seed oil content of canola seed can significantly be increased by introduction of the combinations of LMPs as listed in table 9 of FIG. 8. T2 seeds of plants harbouring the combination of LMPs listed in table 8 were analysed for seed oil content by NIRS. Control plants were non-transgenic segregants grown together with the transgenic plants carrying the combination of LMPs. Only lines with an increase of more than 5% are shown. The p-values shown were calculated using simple t-test.

### DETAILED DESCRIPTION OF THE INVENTION

The present invention may be understood more readily by reference to the following detailed description of the preferred embodiments of the invention and the Examples included therein.

Before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing particular embodiments only and is not intended to be limiting. As used in the specification and in the claims, “a” or “an” can mean one or more, depending upon the context in which it is used. Thus, for example, reference to “a cell” can mean that at least one cell can be utilized.

The term “transgenic” or “recombinant” when used in reference to a cell or an organism (e.g., with regard to a barley plant or plant cell) refers to a cell or organism which contains a transgene, or whose genome has been altered by the introduction of a transgene. A transgenic organism or tissue may comprise one or more transgenic cells. Preferably, the organism or tissue is substantially consisting of transgenic cells (i.e., more than 80%, preferably 90%, more preferably 95%, most preferably 99% of the cells in said organism or tissue are transgenic). The term “transgene” as used herein refers to any nucleic acid sequence, which is introduced into the genome of a cell or which has been manipulated by experimental manipulations by man. Preferably, said sequence is resulting in a genome which is different from a naturally occurring organism (e.g., said sequence, if endogenous to said organism, is introduced into a location different from its natural location, or its copy number is increased or decreased). A transgene may be an “endogenous DNA sequence”, “an “exogenous DNA sequence” (e.g., a foreign gene), or a “heterologous DNA sequence”. The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

The terms “heterologous nucleic acid sequence” or “heterologous DNA” are used interchangeably to refer to a nucleotide sequence, which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. A promoter, transcription regulating sequence or other genetic element is considered to be “heterologous” in relation to another sequence (e.g., encoding a marker sequence or am agronomically relevant trait) if said two sequences are not combined or differently operably linked their natural environment. Preferably, said sequences are not operably linked in their natural environment (i.e. come from different genes). Most preferably, said regulatory sequence is covalently joined and adjacent to a nucleic acid to which it is not adjacent in its natural environment.

One aspect of the invention pertains to combinations of isolated nucleic acid molecules that encode LMP polypeptides or biologically active portions thereof, as well as nucleic acid fragments sufficient for use as hybridization probes or primers for the identification or amplification of an LMP-encoding nucleic acid (e.g., LMP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of a gene: at least about 1000 nucleotides of sequence upstream from the 5′ end of the coding region and at least about 200 nucleotides of sequence downstream from the 3′ end of the coding region of the gene. The nucleic acid molecule can be single-stranded or double-stranded, but preferably is double-stranded DNA. An “isolated” nucleic acid molecule is one, which is substantially separated from other nucleic acid molecules, which are present in the natural source of the nucleic acid. Preferably, an “isolated” nucleic acid is substantially free of sequences that naturally flank the nucleic acid (i.e., sequences located at the 5′ and 3′ ends of the nucleic acid) in the genomic DNA of the organism, from which the nucleic acid is derived. For example, in various embodiments, the isolated LMP nucleic acid molecule can contain less than about 5 kb, 4 kb, 3 kb, 2 kb, 1 kb, 0.5 kb or 0.1 kb of nucleotide sequences, which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule consisting of a combination of isolated nucleotide sequences of Appendix A, or a portion thereof, can be constructed using standard molecular biology techniques and the sequence information provided herein. For example, an Arabidopsis thaliana or Physcomitrella patens, Brassica napus, Glycine max or Linum usitatissimum LMP cDNA can be isolated from an Arabidopsis thaliana or Physcomitrella patens, Brassica napus, Glycine max or Linum usitatissimum library using all or portion of one of the sequences of Appendix A as a hybridization probe and standard hybridization techniques (e.g., as described in Sambrook et al. 1989, Molecular Cloning: A Laboratory Manual. 2nd, ed., Cold Spring Harbor Laboratory, Cold Spring Harbor Laboratory Press, Cold Spring Harbor, N.Y.). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from plant cells (e.g., by the guanidinium-thiocyanate extraction procedure of Chirgwin et al. 1979, Biochemistry 18:5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A and may contain restriction enzyme sites or sites for ligase independent cloning to construct the combinations described by this invention. A nucleic acid of the invention can be amplified using cDNA or, alternatively, genomic DNA, as a template and appropriate oligonucleotide primers according to standard PCR amplification techniques. The nucleic acids so amplified can be cloned into an appropriate vector in the combinations described by the present invention or variations thereof and characterized by DNA sequence analysis. Furthermore, oligonucleotides corresponding to an LMP nucleotide sequence can be prepared by standard synthetic techniques, e.g., using an automated DNA synthesizer.

In another preferred embodiment, an isolated nucleic acid molecule included in a combination of the invention comprises a nucleic acid molecule, which is a complement of one of the nucleotide sequences shown in Appendix A, or a portion thereof. A nucleic acid molecule, which is complementary to one or more of the nucleotide sequences shown in Appendix A, is one which is sufficiently complementary to one or more of the nucleotide sequences shown in Appendix A, such that it can hybridize to one or more of the nucleotide sequences shown in Appendix A, thereby forming a stable duplex.

In still another preferred embodiment, an isolated nucleic acid molecule in the combinations of the invention comprises a nucleotide sequence, which is at least about 50-60%, preferably at least about 60-70%, more preferably at least about 70-80%, 80-90%, or 90-95%, and even more preferably at least about 95%, 96%, 97%, 98%, 99% or more homologous to one or more nucleotide sequence shown in Appendix A, or a portion thereof. In an additional preferred embodiment, an isolated nucleic acid molecule in the combinations of the invention comprises a nucleotide sequence which hybridizes, e.g., hybridizes under stringent conditions, to one or more of the nucleotide sequences shown in Appendix A, or a portion thereof.

For the purposes of the invention hybridization means preferably hybridization under conditions equivalent to hybridization in 7% sodium dodecyl sulfate (SDS), 0.5 M NaPO4, 1 mM EDTA at 50° C. with washing in 2×SSC, 0.1% SDS at 50° C., more desirably in 7% sodium dodecyl sulfate (SDS), 0.5 M NaPO4, 1 mM EDTA at 50° C. with washing in 1×SSC, 0.1% SDS at 50° C., more desirably still in 7% sodium dodecyl sulfate (SDS), 0.5 M NaPO4, 1 mM EDTA at 50° C. with washing in 0.5×SSC, 0.1% SDS at 50° C., preferably in 7% sodium dodecyl sulfate (SDS), 0.5 M NaPO4, 1 mM EDTA at 50° C. with washing in 0.1×SSC, 0.1% SDS at 50° C., more preferably in 7% sodium dodecyl sulfate (SDS), 0.5 M NaPO4, 1 mM EDTA at 50° C. with washing in 0.1×SSC, 0.1% SDS at 65° C. to a nucleic acid comprising 50 to 200 or more consecutive nucleotides.

A further preferred, non-limiting example of stringent hybridization conditions includes washing with a solution having a salt concentration of about 0.02 molar at pH 7 at about 60° C.

Moreover, the nucleic acid molecule in the combinations of the invention can comprise only a portion of the coding region of one of the sequences in Appendix A, for example a fragment, which can be used as a probe or primer or a fragment encoding a biologically active portion of an LMP. The nucleotide sequences determined from the cloning of the LMP Arabidopsis thaliana or Physcomitrella patens, allows for the generation of probes and primers designed for use in identifying and/or cloning LMP homologues in other cell types and organisms, as well as LMP homologues from other plants or related species. Therefore this invention also provides compounds comprising the combinations of nucleic acids disclosed herein, or fragments thereof. These compounds include the nucleic acid combinations attached to a moiety. These moieties include, but are not limited to, detection moieties, hybridization moieties, purification moieties, delivery moieties, reaction moieties, binding moieties, and the like. The probe/primer typically comprises substantially purified oligonucleotide. The oligonucleotide typically comprises a region of nucleotide sequence that hybridizes under stringent conditions to at least about 12, preferably about 25, more preferably about 40, 50, or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone LMP homologues for the combinations described by this inventions or variations thereof. Probes based on the LMP nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which express an LMP, such as by measuring a level of an LMP-encoding nucleic acid in a sample of cells, e.g., detecting LMP mRNA levels, or determining whether a genomic LMP gene has been mutated or deleted.

In one embodiment, the nucleic acid molecule of the invention encodes a combination of proteins or portions thereof, which include amino acid sequences, which are sufficiently homologous to an amino acid encoded by a sequence of Appendix A, such that the protein or portion thereof maintains the same or a similar function as the wild-type protein. As used herein, the language “sufficiently homologous” refers to proteins or portions thereof, which have amino acid sequences, which include a minimum number of identical or equivalent (e.g., an amino acid residue, which has a similar side chain as an amino acid residue in one of the ORFs of a sequence of Appendix A) amino acid residues to an amino acid sequence, such that the protein or portion thereof is able to participate in the metabolism of compounds necessary for the production of seed storage compounds in plants, construction of cellular membranes in microorganisms or plants, or in the transport of molecules across these membranes. Examples of LMP-encoding nucleic acid sequences are set forth in Appendix A.

As altered or increased sugar and/or fatty acid production is a general trait wished to be inherited into a wide variety of plants like maize, wheat, rye, oat, triticale, rice, barley, soybean, peanut, cotton, canola, manihot, pepper, sunflower, sugar beet, and tagetes, solanaceous plants like potato, tobacco, eggplant, and tomato, Vicia species, pea, alfalfa, bushy plants (coffee, cacao, tea), Salix species, trees (oil palm, coconut) and perennial grasses and forage crops, these crop plants are also preferred target plants for genetic engineering as one further embodiment of the present invention.

Portions of proteins encoded by the LMP nucleic acid molecules of the invention are preferably biologically active portions of one of the LMPs. As used herein, the term “biologically active portion of an LMP” is intended to include a portion, e.g., a domain/motif, of an LMP that participates in the metabolism of compounds necessary for the biosynthesis of seed storage lipids, or the construction of cellular membranes in microorganisms or plants, or in the transport of molecules across these membranes, or has an activity as set forth in Table 4. To determine whether an LMP or a biologically active portion thereof can participate in the metabolism of compounds necessary for the production of seed storage compounds and cellular membranes, an assay of enzymatic activity may be performed. Such assay methods are well known to those skilled in the art, and as described in Example 14 of the Exemplification.

Biologically active portions of an LMP include peptides comprising amino acid sequences derived from the amino acid sequence of an LMP (e.g., an amino acid sequence encoded by a nucleic acid of Appendix A or the amino acid sequence of a protein homologous to an LMP, which include fewer amino acids than a full length LMP or the full length protein which is homologous to an LMP) and exhibit at least one activity of an LMP. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an LMP. Moreover, other biologically active portions, in which other regions of the protein are deleted, can be prepared by recombinant techniques and evaluated for one or more of the activities described herein. Preferably, the biologically active portions of an LMP include one or more selected domains/motifs or portions thereof having biological activity.

Additional nucleic acid fragments encoding biologically active portions of an LMP can be prepared by isolating a portion of one of the sequences, expressing the encoded portion of the LMP or peptide (e.g., by recombinant expression in vitro) and assessing the activity of the encoded portion of the LMP or peptide.

The invention further encompasses combinations of nucleic acid molecules that differ from one of the nucleotide sequences shown in Appendix A (and portions thereof) due to degeneracy of the genetic code and thus encode the same LMP as that encoded by the nucleotide sequences shown in Appendix A. In a further embodiment, the combinations of nucleic acid molecule of the invention encode one or more full-length proteins, which are substantially homologous to an amino acid sequence of a polypeptide encoded by an open reading frame shown in Appendix A. In one embodiment, the full-length nucleic acid or protein, or fragment of the nucleic acid or protein, is from Arabidopsis thaliana or Physcomitrella patens.

In addition to the Arabidopsis thaliana or Physcomitrella patens LMP nucleotide sequences shown in Appendix A, it will be appreciated by those skilled in the art that DNA sequence polymorphisms that lead to changes in the amino acid sequences of LMPs may exist within a population Arabidopsis thaliana or Physcomitrella patens population). Such genetic polymorphism in the LMP gene may exist among individuals within a population due to natural variation. As used herein, the terms “gene” and “recombinant gene” refer to nucleic acid molecules comprising an open reading frame encoding an LMP, preferably an Arabidopsis thaliana or Physcomitrella patens LMP. Such natural variations can typically result in 1-40% variance in the nucleotide sequence of the LMP gene. Any and all such nucleotide variations and resulting amino acid polymorphisms in LMP that are the result of natural variation and that do not alter the functional activity of LMPs are intended to be within the scope of the invention.

The invention further encompasses combinations of nucleic acid molecules corresponding to natural variants and non-Arabidopsis thaliana or Physcomitrella patens orthologs of the Arabidopsis thaliana or Physcomitrella patens LMP nucleic acid sequence shown in Appendix A. Nucleic acid molecules corresponding to natural variants and non-Arabidopsis thaliana or Physcomitrella patens orthologs of the Arabidopsis thaliana or Physcomitrella patens LMP cDNA described in Appendix A can be isolated based on their homology to Arabidopsis thaliana or Physcomitrella patens LMP nucleic acid shown in Appendix A using the Arabidopsis thaliana or Physcomitrella patens cDNA, or a portion thereof, as a hybridization probe according to standard hybridization techniques under stringent hybridization conditions. As used herein, the term “orthologs” refers to two nucleic acids from different species, but that have evolved from a common ancestral gene by speciation. Normally, orthologs encode proteins having the same or similar functions. Accordingly, in another embodiment, an isolated nucleic acid molecule is at least 15 nucleotides in length and hybridizes under stringent conditions to the nucleic acid molecule comprising a nucleotide sequence of Appendix A. In other embodiments, the nucleic acid is at least 30, 50, 100, 250, or more nucleotides in length. As used herein, the term “hybridizes under stringent conditions” is intended to describe conditions for hybridization and washing, under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y., 1989: 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule that hybridizes under stringent conditions to a sequence of Appendix A corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring variants of the LMP sequence that may exist in the population, the skilled artisan will further appreciate that changes can be introduced by mutation into a nucleotide sequence of Appendix A, thereby leading to changes in the amino acid sequence of the encoded LMP, without altering the functional ability of the LMP. For example, nucleotide substitutions leading to amino acid substitutions at “non-essential” amino acid residues can be made in a sequence of Appendix A. A “non-essential” amino acid residue is a residue that can be altered from the wild-type sequence of one of the LMPs (Appendix A) without altering the activity of said LMP, whereas an “essential” amino acid residue is required for LMP activity. Other amino acid residues, however, (e.g., those that are not conserved or only semi-conserved in the domain having LMP activity) may not be essential for activity and thus are likely to be amenable to alteration without altering LMP activity.

Accordingly, another aspect of the invention pertains to nucleic acid molecules encoding LMPs that contain changes in amino acid residues that are not essential for LMP activity. Such LMPs differ in amino acid sequence from a sequence yet retain at least one of the LMP activities described herein. In one embodiment, the isolated nucleic acid molecule comprises a nucleotide sequence encoding a protein, wherein the protein comprises an amino acid sequence at least about 50% homologous to an amino acid sequence encoded by a nucleic acid of Appendix A and is capable of participation in the metabolism of compounds necessary for the production of seed storage compounds in Brassica napus, Glycine max or Linum usitatissimum, or cellular membranes, or has one or more activities set forth in Table 4. Preferably, the protein encoded by the nucleic acid molecule is at least about 50-60% homologous to one of the sequences encoded by a nucleic acid of Appendix A, more preferably at least about 60-70% homologous to one of the sequences encoded by a nucleic acid of Appendix A, even more preferably at least about 70-80%, 80-90%, 90-95% homologous to one of the sequences encoded by a nucleic acid of Appendix A, and most preferably at least about 96%, 97%, 98%, or 99% homologous to one of the sequences encoded by a nucleic acid of Appendix A.

To determine the percent homology of two amino acid sequences (e.g., one of the sequences encoded by a nucleic acid of Appendix A and a mutant form thereof), or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences encoded by a nucleic acid of Appendix A) is occupied by the same amino acid residue or nucleotide as the corresponding position in the other sequence (e.g., a mutant form of the sequence selected from the polypeptide encoded by a nucleic acid of Appendix A), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100). The sequence identity can be generally based on any one of the full length sequences of Appendix A as 100%.

For the purposes of the invention, the percent sequence identity between two nucleic acid or polypeptide sequences is determined using the Vector NTI 7.0 (PC) software package (InforMax, 7600 Wisconsin Ave., Bethesda, Md. 20814). A gap-opening penalty of 15 and a gap extension penalty of 6.66 are used for determining the percent identity of two nucleic acids. A gap-opening penalty of 10 and a gap extension penalty of 0.1 are used for determining the percent identity of two polypeptides. All other parameters are set at the default settings. For purposes of a multiple alignment (Clustal W algorithm), the gap-opening penalty is 10, and the gap extension penalty is 0.05 with blosum62 matrix. It is to be understood that for the purposes of determining sequence identity when comparing a DNA sequence to an RNA sequence, a thymidine nucleotide sequence is equivalent to an uracil nucleotide.

An isolated nucleic acid molecule encoding an LMP homologous to a protein sequence encoded by a nucleic acid of Appendix A can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in an LMP is preferably replaced with another amino acid residue from the same side chain family. Alternatively, in another embodiment, mutations can be introduced randomly along all or part of an LMP coding sequence, such as by saturation mutagenesis, and the resultant mutants can be screened for an LMP activity described herein to identify mutants that retain LMP activity. Following mutagenesis of one of the sequences of Appendix A, the encoded protein can be expressed recombinantly, and the activity of the protein can be determined using, for example, assays described herein (see Examples 11-13 of the Exemplification).

Combinations of LMPs are preferably produced by recombinant DNA techniques. For example, one or more nucleic acid molecule encoding the protein is cloned into an expression vector (as described above), the expression vector is introduced into a host cell (as described herein), and the LMPs are expressed in the host cell. The LMPs can then be isolated from the cells by an appropriate purification scheme using standard protein purification techniques. Alternative to recombinant expression, one or more LMP or peptide thereof can be synthesized chemically using standard peptide synthesis techniques. Moreover, native LMPs can be isolated from cells, for example using an anti-LMP antibody, which can be produced by standard techniques utilizing an LMP or fragment thereof of this invention.

The invention also provides combinations of LMP chimeric or fusion proteins. As used herein, an LMP “chimeric protein” or “fusion protein” comprises an LMP polypeptide operatively linked to a non-LMP polypeptide. An “LMP polypeptide” refers to a polypeptide having an amino acid sequence corresponding to an LMP, whereas a “non-LMP polypeptide” refers to a polypeptide having an amino acid sequence corresponding to a protein which is not substantially homologous to the LMP, e.g., a protein which is different from the LMP, and which is derived from the same or a different organism. Within the fusion protein, the term “operatively linked” is intended to indicate that the LMP polypeptide and the non-LMP polypeptide are fused to each other so that both sequences fulfill the proposed function attributed to the sequence used. The non-LMP polypeptide can be fused to the N-terminus or C-terminus of the LMP polypeptide. For example, in one embodiment, the fusion protein is a GST-LMP (glutathione S-transferase) fusion protein in which the LMP sequences are fused to the C-terminus of the GST sequences. Such fusion proteins can facilitate the purification of recombinant LMPs. In another embodiment, the fusion protein is an LMP containing a heterologous signal sequence at its N-terminus. In certain host cells (e.g., mammalian host cells), expression and/or secretion of an LMP can be increased through use of a heterologous signal sequence.

Preferably, a combination of LMP chimeric or fusion proteins of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide). An LMP-encoding nucleic acid can be cloned into such an expression vector such that the fusion moiety is linked in-frame to the LMP.

In addition to the nucleic acid molecules encoding LMPs described above, another aspect of the invention pertains to combinations of isolated nucleic acid molecules that are antisense thereto. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire LMP coding strand, or to only a portion thereof. In one embodiment, an antisense nucleic acid molecule is antisense to a “coding region” of the coding strand of a nucleotide sequence encoding an LMP. The term “coding region” refers to the region of the nucleotide sequence comprising codons that are translated into amino acid residues. In another embodiment, the antisense nucleic acid molecule is antisense to a “noncoding region” of the coding strand of a nucleotide sequence encoding LMP. The term “noncoding region” refers to 5′ and 3′ sequences that flank the coding region that are not translated into amino acids (i.e., also referred to as 5′ and 3′ untranslated regions).

Given the coding strand sequences encoding LMP disclosed herein (e.g., the sequences set forth in Appendix A), combinations of antisense nucleic acids of the invention can be designed according to the rules of Watson and Crick base pairing. The antisense nucleic acid molecule can be complementary to the entire coding region of LMP mRNA, but more preferably is an oligonucleotide that is antisense to only a portion of the coding or noncoding region of LMP mRNA. For example, the antisense oligonucleotide can be complementary to the region surrounding the translation start site of LMP mRNA. An antisense oligonucleotide can be, for example, about 5, 10, 15, 20, 25, 30, 35, 40, 45, or 50 nucleotides in length. An antisense or sense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylamino-methyl-2-thiouridine, 5-carboxymethylaminomethyluracil, dihydro-uracil, beta-D-galactosylqueosine, inosine, N-6-isopentenyladenine, 1-methyl-guanine, 1-methylinosine, 2,2-dimethylguanine, 2-methyladenine, 2-methylguanine, 3-methylcytosine, 5-methyl-cytosine, N-6-adenine, 7-methylguanine, 5-methyl-aminomethyluracil, 5-methoxyamino-methyl-2-thiouracil, beta-D-mannosylqueosine, 5′-methoxycarboxymethyl-uracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diamino-purine. Alternatively, the antisense nucleic acid can be produced biologically using an expression vector, into which a nucleic acid has been subcloned in an antisense orientation (i.e., RNA transcribed from the inserted nucleic acid will be of an antisense orientation to a target nucleic acid of interest, described further in the following subsection).

In another variation of the antisense technology, a double-strand, interfering, RNA construct can be used to cause a down-regulation of the LMP mRNA level and LMP activity in transgenic plants. This requires transforming the plants with a chimeric construct containing a portion of the LMP sequence in the sense orientation fused to the antisense sequence of the same portion of the LMP sequence. A DNA linker region of variable length can be used to separate the sense and antisense fragments of LMP sequences in the construct.

Combinations of the antisense nucleic acid molecules of the invention are typically administered to a cell or generated in situ, such that they hybridize with or bind to cellular mRNA and/or genomic DNA encoding an LMP to thereby inhibit expression of the protein, e.g., by inhibiting transcription and/or translation. The hybridization can be by conventional nucleotide complementarity to form a stable duplex, or, for example, in the case of an antisense nucleic acid molecule, which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody, which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic, including plant promoters are preferred.

In yet another embodiment, the combinations of antisense nucleic acid molecules of the invention are -anomeric nucleic acid molecules. An anomeric nucleic acid molecule forms specific double-stranded hybrids with complementary RNA, in which, contrary to the usual units, the strands run parallel to each other (Gaultier et al. 1987, Nucleic Acids Res. 15:6625-6641). The antisense nucleic acid molecule can also comprise a 2′-o-methyl-ribonucleotide (Inoue et al. 1987, Nucleic Acids Res. 15:6131-6148) or a chimeric RNA-DNA analogue (Inoue et al. 1987, FEBS Lett. 215:327-330).

In still another embodiment, a combination containing an antisense nucleic acid of the invention contains a ribozyme. Ribozymes are catalytic RNA molecules with ribonuclease activity, which are capable of cleaving a single-stranded nucleic acid, such as an mRNA, to which they have a complementary region. Thus, ribozymes (e.g., hammerhead ribozymes (described in Haselhoff & Gerlach 1988, Nature 334:585-591)) can be used to catalytically cleave LMP mRNA transcripts to thereby inhibit translation of LMP mRNA. A ribozyme having specificity for an LMP-encoding nucleic acid can be designed based upon the nucleotide sequence of an LMP cDNA disclosed herein (i.e., Bn01 in Appendix A) or on the basis of a heterologous sequence to be isolated according to methods taught in this invention. For example, a derivative of a Tetrahymena L-19 IVS RNA can be constructed, in which the nucleotide sequence of the active site is complementary to the nucleotide sequence to be cleaved in an LMP-encoding mRNA (see, e.g., Cech et al., U.S. Pat. No. 4,987,071 and Cech et al., U.S. Pat. No. 5,116,742). Alternatively, LMP mRNA can be used to select a catalytic RNA having a specific ribonuclease activity from a pool of RNA molecules (see, e.g., Bartel, D. & Szostak J. W. 1993, Science 261:1411-1418).

Alternatively, LMP gene expression of one or more genes of the combinations of this invention can be inhibited by targeting nucleotide sequences complementary to the regulatory region of an LMP nucleotide sequence (e.g., an LMP promoter and/or enhancers) to form triple helical structures that prevent transcription of an LMP gene in target cells (See generally, Helene C. 1991, Anticancer Drug Des. 6:569-84; Helene C. et al. 1992, Ann. N.Y. Acad. Sci. 660:27-36; and Maher, L. J. 1992, Bioassays 14:807-15).

Another aspect of the invention pertains to vectors, preferably expression vectors, containing a combination of nucleic acids encoding LMPs (or a portion thereof. As used herein, the term “vector” refers to a nucleic acid molecule capable of transporting another nucleic acid, to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell, into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes, to which they are operatively linked. Such vectors are referred to herein as “expression vectors.” In general, expression vectors of utility in recombinant DNA techniques are often in the form of plasmids. In the present specification, “plasmid,” and “vector” can be used inter-changeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a combination of nucleic acids of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence and both sequences are fused to each other so that each fulfills its proposed function (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel; Gene Expression Technology: Methods in Enzymology 185, Academic Press, San Diego, Calif. (1990) or see: Gruber and Crosby, in: Methods in Plant Molecular Biology and Biotechnolgy, CRC Press, Boca Raton, Fla., eds.: Glick & Thompson, Chapter 7, 89-108 including the references therein. Regulatory sequences include those that direct constitutive expression of a nucleotide sequence in many types of host cell and those that direct expression of the nucleotide sequence only in certain host cells or under certain conditions. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., LMPs, mutant forms of LMPs, fusion proteins, etc.).

The recombinant expression vectors of the invention can be designed for expression of combinations of LMPs in prokaryotic or eukaryotic cells. For example, LMP genes can be expressed in bacterial cells, insect cells (using baculovirus expression vectors), yeast and other fungal cells (see Romanos M. A. et al. 1992, Foreign gene expression in yeast: a review, Yeast 8:423-488; van den Hondel, C. A. M. J. J. et al. 1991, Heterologous gene expression in filamentous fungi, in: More Gene Manipulations in Fungi, Bennet & Lasure, eds., p. 396-428:Academic Press: an Diego; and van den Hondel & Punt 1991, Gene transfer systems and vector development for filamentous fungi, in: Applied Molecular Genetics of Fungi, Peberdy et al., eds., p. 1-28, Cambridge University Press: Cambridge), algae (Falciatore et al. 1999, Marine Biotechnology 1:239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in WO 98/01572 and multicellular plant cells (see Schmidt & Willmitzer 1988, High efficiency Agrobacterium tumefaciens-mediated transformation of Arabidopsis thaliana leaf and cotyledon plants, Plant Cell Rep.: 583-586); Plant Molecular Biology and Biotechnology, C Press, Boca Raton, Fla., chapter 6/7, S.71-119 (1993); White, Jenes et al., Techniques for Gene Transfer, in: Transgenic Plants, Vol. 1, Engineering and Utilization, eds.: Kung and Wu, Academic Press 1993, 128-43; Potrykus 1991, Annu. Rev. Plant Physiol. Plant Mol. Biol. 42:205-225 (and references cited therein) or mammalian cells. Suitable host cells are discussed further in Goeddel, Gene Expression Technology: Methods in Enzymology 185, Academic Press, San Diego, Calif. 1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve one or more of the following purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, and enterokinase.

Typical fusion expression vectors include pGEX (Pharmacia Biotech Inc; Smith & Johnson 1988, Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein. In one embodiment, the coding sequence of the LMP is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant LMP unfused to GST can be recovered by cleavage of the fusion protein with thrombin.

Examples of suitable inducible non-fusion E. coli expression vectors include pTrc (Amann et al. 1988, Gene 69:301-315) and pET 11d (Studier et al. 1990, Gene Expression Technology: Methods in Enzymology 185, Academic Press, San Diego, Calif. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression is to express the protein in a host bacteria with an impaired capacity to proteolytically cleave the recombinant protein (Gottesman S. 1990, Gene Expression Technology: Methods in Enzymology 185:119-128, Academic Press, San Diego, Calif.). Another strategy is to alter the nucleic acid sequence of the nucleic acid to be inserted into an expression vector so that the individual codons for each amino acid are those preferentially utilized in the bacterium chosen for expression (Wada et al. 1992, Nucleic Acids Res. 20:2111-2118). Such alteration of nucleic acid sequences of the invention can be carried out by standard DNA synthesis techniques.

In another embodiment, the LMP combination expression vector is a yeast expression vector. Examples of vectors for expression in yeast S. cerevisiae include pYepSec1 (Baldari et al. 1987, Embo J. 6:229-234), pMFa (Kurjan & Herskowitz 1982, Cell 30:933-943), pJRY88 (Schultz et al. 1987, Gene 54:113-123), and pYES2 (Invitrogen Corporation, San Diego, Calif.). Vectors and methods for the construction of vectors appropriate for use in other fungi, such as the filamentous fungi, include those detailed in: van den Hondel & Punt 1991, “Gene transfer systems and vector development for filamentous fungi,” in: Applied Molecular Genetics of Fungi, Peberdy et al., eds., p. 1-28, Cambridge University Press: Cambridge.

Alternatively, the combinations of LMPs of the invention can be expressed in insect cells using baculovirus expression vectors. Baculovirus vectors available for expression of proteins in cultured insect cells (e.g., Sf 9 cells) include the pAc series (Smith et al. 1983, Mol. Cell. Biol. 3:2156-2165) and the pVL series (Lucklow & Summers 1989, Virology 170:31-39).

In yet another embodiment, a combination of nucleic acids of the invention is expressed in mammalian cells using a mammalian expression vector. Examples of mammalian expression vectors include pCDM8 (Seed 1987, Nature 329:840) and pMT2PC (Kaufman et al. 1987, EMBO J. 6:187-195). When used in mammalian cells, the expression vectors control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells see chapters 16 and 17 of Sambrook, Fritsh and Maniatis, Molecular Cloning: A Laboratory Manual. 2nd, ed., Cold Spring Harbor Laboratory, Cold Spring Harbor Laboratory Press, Cold Spring Harbor, N.Y., 1989.

In another embodiment, a combination of the LMPs of the invention may be expressed in unicellular plant cells (such as algae, see Falciatore et al. (1999, Marine Biotechnology 1:239-251 and references therein) and plant cells from higher plants (e.g., the spermatophytes, such as crop plants). Examples of plant expression vectors include those detailed in: Becker, Kemper, Schell and Masterson (1992, “New plant binary vectors with selectable markers located proximal to the left border,” Plant Mol. Biol. 20:1195-1197) and Bevan (1984, “Binary Agrobacterium vectors for plant transformation,” Nucleic Acids Res. 12:8711-8721; Vectors for Gene Transfer in Higher Plants; in: Transgenic Plants, Vol. 1, Engineering and Utilization, eds.: Kung und R. Wu, Academic Press, 1993, S. 15-38).

A plant expression cassette preferably contains regulatory sequences capable to drive gene expression in plant cells, and which are operably linked so that each sequence can fulfill its function such as termination of transcription, including polyadenylation signals. Preferred polyadenylation signals are those originating from Agrobacterium tumefaciens t-DNA such as the gene 3 known as octopine synthase of the Ti-plasmid pTiACH5 (Gielen et al. 1984, EMBO J. 3:835) (SEQ ID No. 16) or functional equivalents thereof. but also all other terminators functionally active in plants are suitable (e.g. Seq ID No. 14, 15 and 17).

As plant gene expression is very often not limited on transcriptional levels a plant expression cassette preferably contains other operably-linked sequences, like translational enhancers such as the overdrive-sequence containing the 5′-untranslated leader sequence from tobacco mosaic virus enhancing the protein per RNA ratio (Gallie et al. 1987, Nucleic Acids Res. 15:8693-8711).

Plant gene expression has to be operably linked to an appropriate promoter conferring gene expression in a timely, cell or tissue specific manner. Preferred are promoters driving constitutive expression (Benfey et al. 1989, EMBO J. 8:2195-2202) like those derived from plant viruses like the 35S CAMV (Franck et al. 1980, Cell 21:285-294), the 19S CaMV (see also U.S. Pat. No. 5,352,605 and WO 84/02913) or the ptxA promoter SEQ ID No. 9 (Bown, D. P. PhD thesis (1992) Department of Biological Sciences, University of Durham, Durham, U.K) or plant promoters like those from Rubisco small subunit described in U.S. Pat. No. 4,962,028. Even more preferred are seed-specific promoters driving expression of LMP proteins during all or selected stages of seed development. Seed-specific plant promoters are known to those of ordinary skill in the art and are identified and characterized using seed-specific mRNA libraries and expression profiling techniques. Seed-specific promoters include the napin-gene promoter from rapeseed (U.S. Pat. No. 5,608,152), the USP-promoter from Vicia faba (Baeumlein et al. 1991, Mol. Gen. Genetics 225:459-67) SEQ ID No. 10, the oleosin-promoter from Arabidopsis (WO 98/45461), the phaseolin-promoter from Phaseolus vulgaris (U.S. Pat. No. 5,504,200), the Bce4-promoter from Brassica (WO9113980) or the legumin B4 promoter (LeB4; Baeumlein et al. 1992, Plant J. 2:233-239) SEQ ID No. 11 & 12), as well as promoters conferring seed specific expression in monocot plants like maize, barley, wheat, rye, rice etc. Suitable promoters to note are the lpt2 or lpt1-gene promoter from barley (WO 95/15389 and WO 95/23230) or those described in WO 99/16890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, and the rye secalin gene).

Plant gene expression can also be facilitated via an inducible promoter (for a review see Gatz 1997, Annu. Rev. Plant Physiol. Plant Mol. Biol. 48:89-108). Chemically inducible promoters are especially suitable if gene expression is desired in a time specific manner. Examples for such promoters are a salicylic acid inducible promoter (WO 95/19443), a tetracycline inducible promoter (Gatz et al. 1992, Plant J. 2:397-404) and an ethanol inducible promoter (WO 93/21334).

Promoters responding to biotic or abiotic stress conditions are also suitable promoters such as the pathogen inducible PRP1-gene promoter (Ward et al., 1993, Plant Mol. Biol. 22:361-366), the heat inducible hsp80-promoter from tomato (U.S. Pat. No. 5,187,267), cold inducible alpha-amylase promoter from potato (WO 96/12814) or the wound-inducible pinII-promoter (EP 375091).

Other preferred sequences for use in plant gene expression cassettes are targeting-sequences necessary to direct the gene-product in its appropriate cell compartment (for review see Kermode 1996, Crit. Rev. Plant Sci. 15:285-423 and references cited therein) such as the vacuole, the nucleus, all types of plastids like amyloplasts, chloroplasts, chromoplasts, the extracellular space, mitochondria, the endoplasmic reticulum, oil bodies, peroxisomes, and other compartments of plant cells. Also especially suited are promoters that confer plastid-specific gene expression, as plastids are the compartment where precursors and some end products of lipid biosynthesis are synthesized. Suitable promoters such as the viral RNA-polymerase promoter are described in WO 95/16783 and WO 97/06250 and the clpP-promoter from Arabidopsis described in WO 99/46394.

The invention further provides a recombinant expression vector comprising a combination of DNA molecules of the invention cloned into the expression vector in an antisense orientation. That is, the DNA molecule is operatively linked to a regulatory sequence in a manner that allows for expression (by transcription of the DNA molecule) of an RNA molecule that is antisense to LMP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus, in which antisense nucleic acids are produced under the control of a high efficiency regulatory region, the activity of which can be determined by the cell type, into which the vector is introduced. For a discussion of the regulation of gene expression using antisense genes see Weintraub et al. (1986, Antisense RNA as a molecular tool for genetic analysis, Reviews—Trends in Genetics, Vol. 1) and Mol et al. (1990, FEBS Lett. 268:427-430).

Another aspect of the invention pertains to host cells into which a recombinant expression vector of the invention has been introduced. The terms “host cell” and “recombinant host cell” are used interchangeably herein. It is to be understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a combination of LMPs can be expressed in bacterial cells, insect cells, fungal cells, mammalian cells (such as Chinese hamster ovary cells (CHO) or COS cells), algae, ciliates, or plant cells. Other suitable host cells are known to those skilled in the art.

Vector DNA can be introduced into prokaryotic or eukaryotic cells via conventional transformation or transfection techniques. As used herein, the terms “transformation” and “transfection,” “conjugation,” and “transduction” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells including plant cells can be found in Sambrook et al. (1989, Molecular Cloning: A Laboratory Manual. 2nd, ed., Cold Spring Harbor Laboratory, Cold Spring Harbor Laboratory Press, Cold Spring Harbor, N.Y.) and other laboratory manuals such as Methods in Molecular Biology 1995, Vol. 44, Agrobacterium protocols, ed: Gartland and Davey, Humana Press, Totowa, N.J.

For stable transfection of mammalian and plant cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those that confer resistance to drugs, such as G418, hygromycin, kanamycin, and methotrexate or in plants that confer resistance towards an herbicide, such as glyphosate or glufosinate. A nucleic acid encoding a selectable marker can be introduced into a host cell on the same vector as that encoding a combination of LMPs or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared that contains a combination of at least a portion of an LMP gene, into which a deletion, addition or substitution has been introduced to thereby alter, e.g., functionally disrupt, the LMP gene. Preferably, this LMP gene is an Arabidopsis thaliana or Physcomitrella patens LMP gene, but it can be a homologue from a related plant or even from a mammalian, yeast, or insect source. In a preferred embodiment, the vector is designed such that, upon homologous recombination, the endogenous LMP gene is functionally disrupted (i.e., no longer encodes a functional protein; also referred to as a knock-out vector). Alternatively, the vector can be designed such that, upon homologous recombination, the endogenous LMP gene is mutated or otherwise altered but still encodes functional protein (e.g., the upstream regulatory region can be altered to thereby alter the expression of the endogenous LMP). To create a point mutation via homologous recombination, DNA-RNA hybrids can be used in a technique known as chimeraplasty (Cole-Strauss et al. 1999, Nucleic Acids Res. 27:1323-1330 and Kmiec 1999, American Scientist 87:240-247). Homologous recombination procedures in Arabidopsis thaliana or other crops are also well known in the art and are contemplated for use herein.

In a homologous recombination vector, within the combination of genes coding for LMPs shown in Appendix A the altered portion of the LMP gene is flanked at its 5′ and 3′ ends by additional nucleic acid of the LMP gene to allow for homologous recombination to occur between the exogenous LMP gene carried by the vector and an endogenous LMP gene in a microorganism or plant. The additional flanking LMP nucleic acid is of sufficient length for successful homologous recombination with the endogenous gene. Typically, several hundreds of base pairs up to kilobases of flanking DNA (both at the 5′ and 3′ ends) are included in the vector (see e.g., Thomas & Capecchi 1987, Cell 51:503, for a description of homologous recombination vectors). The vector is introduced into a microorganism or plant cell (e.g., via polyethyleneglycol mediated DNA). Cells in which the introduced LMP gene has homologously recombined with the endogenous LMP gene are selected using art-known techniques.

In another embodiment, recombinant microorganisms can be produced which contain selected systems, which allow for regulated expression of the introduced combinations of genes. For example, inclusion of a combination of one two or more LMP genes on a vector placing it under control of the lac operon permits expression of the LMP gene only in the presence of IPTG. Such regulatory systems are well known in the art.

A host cell of the invention, such as a prokaryotic or eukaryotic host cell in culture can be used to produce (i.e., express) a combination of LMPs. Accordingly, the invention further provides methods for producing LMPs using the host cells of the invention. In one embodiment, the method comprises culturing a host cell of the invention (into which a recombinant expression vector encoding a combination of LMPs has been introduced, or which contains a wild-type or altered LMP gene in it's genome) in a suitable medium until the combination of LMPs is produced.

An isolated LMP or a portion thereof of the invention can participate in the metabolism of compounds necessary for the production of seed storage compounds in Brassica napus, Glycine max or Linum usitatissimum or of cellular membranes, or has one or more of the activities set forth in Table 4. In preferred embodiments, the protein or portion thereof comprises an amino acid sequence which is sufficiently homologous to an amino acid sequence encoded by a nucleic acid of Appendix A such that the protein or portion thereof maintains the ability to participate in the metabolism of compounds necessary for the construction of cellular membranes in Brassica napus, Glycine max or Linum usitatissimum, or in the transport of molecules across these membranes. The portion of the protein is preferably a biologically active portion as described herein. In another preferred embodiment, an LMP of the invention has an amino acid sequence encoded by a nucleic acid of Appendix A. In yet another preferred embodiment, the LMP has an amino acid sequence which is encoded by a nucleotide sequence which hybridizes, e.g., hybridizes under stringent conditions, to a nucleotide sequence of Appendix A. In still another preferred embodiment, the LMP has an amino acid sequence which is encoded by a nucleotide sequence that is at least about 50-60%, preferably at least about 60-70%, more preferably at least about 70-80%, 80-90%, 90-95%, and even more preferably at least about 96%, 97%, 98%, 99%, or more homologous to one of the amino acid sequences encoded by a nucleic acid of Appendix A. The preferred LMPs of the present invention also preferably possess at least one of the LMP activities described herein. For example, a preferred LMP of the present invention includes an amino acid sequence encoded by a nucleotide sequence which hybridizes, e.g., hybridizes under stringent conditions, to a nucleotide sequence of Appendix A, and which can participate in the metabolism of compounds necessary for the construction of cellular membranes in Brassica napus, Glycine max or Linum usitatissimum, or in the transport of molecules across these membranes, or which has one or more of the activities set forth in Table 4.

In other embodiments, the combination of LMPs is substantially homologous to a combination of amino acid sequences encoded by nucleic acids of Appendix A and retain the functional activity of the protein of one of the sequences encoded by a nucleic acid of Appendix A yet differs in amino acid sequence due to natural variation or mutagenesis, as described in detail above. Accordingly the LMP is a protein which comprises an amino acid sequence which is at least about 50-60%, preferably at least about 60-70%, and more preferably at least about 70-80, 80-90, 90-95%, and most preferably at least about 96%, 97%, 98%, 99%, or more homologous to an entire amino acid sequence and which has at least one of the LMP activities described herein. In another embodiment, the invention pertains to a full Arabidopsis thaliana or Physcomitrella patens, Brassica napus, Glycine max or Linum usitatissimum protein which is substantially homologous to an entire amino acid sequence encoded by a nucleic acid of Appendix A.

Dominant negative mutations or trans-dominant suppression can be used to reduce the activity of an LMP in transgenics seeds in order to change the levels of seed storage compounds. To achieve this a mutation that abolishes the activity of the LMP is created and the inactive non-functional LMP gene is overexpressed as part of the combination of this invention in the transgenic plant. The inactive trans-dominant LMP protein competes with the active endogenous LMP protein for substrate or interactions with other proteins and dilutes out the activity of the active LMP. In this way the biological activity of the LMP is reduced without actually modifying the expression of the endogenous LMP gene. This strategy was used by Pontier et al to modulate the activity of plant transcription factors (Pontier D, Miao Z H, Lam E, Plant J 2001 Sep. 27(6): 529-38, Trans-dominant suppression of plant TGA factors reveals their negative and positive roles in plant defense responses).

Homologues of the LMP can be generated for combinations by mutagenesis, e.g., discrete point mutation or truncation of the LMP. As used herein, the term “homologue” refers to a variant form of the LMP that acts as an agonist or antagonist of the activity of the LMP. An agonist of the LMP can retain substantially the same, or a subset, of the biological activities of the LMP. An antagonist of the LMP can inhibit one or more of the activities of the naturally-occurring form of the LMP, by, for example, competitively binding to a downstream or upstream member of the cell membrane component metabolic cascade, which includes the LMP, or by binding to an LMP, which mediates transport of compounds across such membranes, thereby preventing translocation from taking place.

In addition, libraries of fragments of the LMP coding sequences can be used to generate a variegated population of LMP fragments for screening and subsequent selection of homologues of an LMP to be included in combinations as described in table 3. In one embodiment, a library of coding sequence fragments can be generated by treating a double stranded PCR fragment of an LMP coding sequence with a nuclease under conditions, wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived, which encodes N-terminal, C-terminal and internal fragments of various sizes of the LMP.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of LMP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify LMP homologues (Arkin & Yourvan 1992, Proc. Natl. Acad. Sci. USA 89:7811-7815; Delgrave et al. 1993, Protein Engineering 6:327-331).

In another embodiment, cell based assays can be exploited to analyze a variegated LMP library, using methods well known in the art.

The nucleic acid molecules, proteins, protein homologues and fusion proteins for the combinations described herein, and vectors, and host cells described herein can be used in one or more of the following methods: identification of Arabidopsis thaliana or Physcomitrella patens and related organisms; mapping of genomes of organisms related to Arabidopsis thaliana or Physcomitrella patens; identification and localization of Arabidopsis thaliana or Physcomitrella patens sequences of interest; evolutionary studies; determination of LMP regions required for function; modulation of an LMP activity; modulation of the metabolism of one or more cell functions; modulation of the transmembrane transport of one or more compounds; and modulation of seed storage compound accumulation.

The plant Arabidopsis thaliana represents one member of higher (or seed) plants. It is related to other plants such as Brassica napus, Glycine max or Linum usitatissimum which require light to drive photosynthesis and growth. Plants like Arabidopsis thaliana, Brassica napus, Glycine max or Linum usitatissimum share a high degree of homology on the DNA sequence and polypeptide level, allowing the use of heterologous screening of DNA molecules with probes evolving from other plants or organisms, thus enabling the derivation of a consensus sequence suitable for heterologous screening or functional annotation and prediction of gene functions in third species, isolation of the corresponding genes and use of the later in combinations described for the sequences listed in Appendix A.

There are a number of mechanisms by which the alteration of a combination of LMPs of the invention may directly affect the accumulation and/or composition of seed storage compounds. In the case of plants expressing a combination of LMPs, increased transport can lead to altered accumulation of compounds, which ultimately could be used to affect the accumulation of one or more seed storage compounds during seed development. Expression of single genes affecting seed storage compound accumulation and/or solute partitioning within the plant tissue and organs is well known. An example is provided by Mitsukawa et al. (1997, Proc. Natl. Acad. Sci. USA 94:7098-7102), where overexpression of an Arabidopsis high-affinity phosphate transporter gene in tobacco cultured cells enhanced cell growth under phosphate-limited conditions. Phosphate availability also affects significantly the production of sugars and metabolic intermediates (Hurry et al. 2000, Plant J. 24:383-396) and the lipid composition in leaves and roots (Härtel et al. 2000, Proc. Natl. Acad. Sci. USA 97:10649-10654). Likewise, the activity of the plant ACCase has been demonstrated to be regulated by phosphorylation (Savage & Ohlrogge 1999, Plant J. 18:521-527) and alterations in the activity of the kinases and phosphatases (LMPs) that act on the ACCase could lead to increased or decreased levels of seed lipid accumulation. Moreover, the presence of lipid kinase activities in chloroplast envelope membranes suggests that signal transduction pathways and/or membrane protein regulation occur in envelopes (see, e.g., Müller et al. 2000, J. Biol. Chem. 275:19475-19481 and literature cited therein). The ABI1 and ABI2 genes encode two protein serine/threonine phosphatases 2C, which are regulators in abscisic acid signaling pathway, and thereby in early and late seed development (e.g. Merlot et al. 2001, Plant J. 25:295-303). For more examples see also the section “Background of the Invention.”

Throughout this application, various publications are referenced. The disclosures of all of these publications and those references cited within those publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and Examples be considered as exemplary only, with a true scope and spirit of the invention being indicated by the claims included herein.

### EXAMPLES

### Example 1

General Processes—a) General Cloning Processes. Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989, Cold Spring Harbor Laboratory Press: ISBN 0-87969-309-6) or Kaiser, Michaelis and Mitchell (1994, “Methods in Yeast Genetics,” Cold Spring Harbor Laboratory Press: ISBN 0-87969-451-3).

### Example 1

General Processes—b) Chemicals. The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H2O in the following text, from a Milli-Q water system water purification plant (Millipore, Eschborn). Restriction endonucleases, DNA-modifying enzymes and molecular biology kits were obtained from the companies AGS (Heidelberg), Amersham (Braunschweig), Biometra (Göttingen), Roche (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, Netherlands). They were used, if not mentioned otherwise, according to the manufacturer's instructions.

### Example 1

General Processes—c) Plant Material and Growth: Arabidopsis plants. For this study, root material, leaves, siliques and seeds of wild-type and transgenic plants of Arabidopsis thaliana expressing combinations of LMPs as described within this invention were used. Wild type and transgenic Arabidopsis seeds were preincubated for three days in the dark at 4° C. before placing them into an incubator (AR-75, Percival Scientific, Boone, Iowa) at a photon flux density of 60-80 μmol m-2 s-1 and a light period of 16 hours (22° C.), and a dark period of 8 hours (18° C.). All plants were started on half-strength MS medium (Murashige & Skoog, 1962, Physiol. Plant. 15, 473-497), pH 6.2, 2% sucrose and 1.2% agar. Seeds were sterilized for 20 minutes in 20% bleach 0.5% triton X100 and rinsed 6 times with excess sterile water.

### Example 2

Total DNA Isolation from Plants. The details for the isolation of total DNA relate to the working up of 1 gram fresh weight of plant material.

CTAB buffer: 2% (w/v) N-cethyl-N,N,N-trimethylammonium bromide (CTAB); 100 mM Tris HCl pH 8.0; 1.4 M NaCl; 20 mM EDTA. N-Laurylsarcosine buffer: 10% (w/v) N-laurylsarcosine; 100 mM Tris HCl pH 8.0; 20 mM EDTA.

The plant material was triturated under liquid nitrogen in a mortar to give a fine powder and transferred to 2 ml Eppendorf vessels. The frozen plant material was then covered with a layer of 1 ml of decomposition buffer (1 ml CTAB buffer, 100 μl of N-laurylsarcosine buffer, 20 μl of p-mercaptoethanol and 10 μl of proteinase K solution, 10 mg/ml) and incubated at 60° C. for 1 hour with continuous shaking. The homogenate obtained was distributed into two Eppendorf vessels (2 ml) and extracted twice by shaking with the same volume of chloroform/isoamyl alcohol (24:1). For phase separation, centrifugation was carried out at 8000 g and RT for 15 min in each case. The DNA was then precipitated at −70° C. for 30 min using ice-cold isopropanol. The precipitated DNA was sedimented at 4° C. and 10,000 g for 30 min and resuspended in 180 μl of TE buffer (Sambrook et al. 1989, Cold Spring Harbor Laboratory Press: ISBN 0-87969-309-6). For further purification, the DNA was treated with NaCl (1.2 M final concentration) and precipitated again at −70° C. for 30 min using twice the volume of absolute ethanol. After a washing step with 70% ethanol, the DNA was dried and subsequently taken up in 50 μl of H2O+RNAse (50 mg/ml final concentration). The DNA was dissolved overnight at 4° C. and the RNAse digestion was subsequently carried out at 37° C. for 1 h. Storage of the DNA took place at 4° C.

### Example 3

Isolation of Total RNA and poly-(A)+ RNA from Plants—Arabidopsis thaliana. For the investigation of transcripts, both total RNA and poly-(A)+ RNA were isolated.

RNA is isolated from siliques of Arabidopsis plants according to the following procedure:

RNA preparation from Arabidopsis seeds—“hot” extraction:

1. Buffers, enzymes and solution
2M KCl

Proteinase K
Phenol (for RNA)
Chloroform:Isoamylalcohol
(Phenol:chloroform 1:1; pH adjusted for RNA)
4 M LiCl, DEPC-treated
DEPC-treated water
3M NaOAc, pH 5, DEPC-treated
Isopropanol
70% ethanol (made up with DEPC-treated water)
Resuspension buffer: 0.5% SDS, 10 mM Tris pH 7.5, 1 mM EDTA made up with

DEPC-Treated Water as this Solution cannot be DEPC-Treated
Extraction Buffer:
0.2M Na Borate
30 mM EDTA
30 mM EGTA
1% SDS (250 μl of 10% SDS-solution for 2.5 ml buffer)
1% Deoxycholate (25 mg for 2.5 ml buffer)
2% PVPP (insoluble—50 mg for 2.5 ml buffer)
2% PVP 40K (50 mg for 2.5 ml buffer)
10 mM DTT
   100 mM p-Mercaptoethanol (fresh, handle under fume hood—use 35 μl of 14.3M solution for 5 ml buffer)

2. Extraction. Heat extraction buffer up to 80° C. Grind tissue in liquid nitrogen-cooled mortar, transfer tissue powder to 1.5 ml tube. Tissue should be kept frozen until buffer is added so transfer the sample with pre-cooled spatula and keep the tube in liquid nitrogen all time. Add 350 μl preheated extraction buffer (here for 100 mg tissue, buffer volume can be as much as 500 μl for bigger samples) to tube, vortex and heat tube to 80° C. for ˜1 min. Keep then on ice. Vortex sample, grind additionally with electric mortar.

3. Digestion. Add Proteinase K (0.15 mg/100 mg tissue), vortex and keep at 37° C. for one hour.

First Purification. Add 27 μl 2M KCl. Chill on ice for 10 min. Centrifuge at 12.000 rpm for 10 minutes at room temperature. Transfer supernatant to fresh, RNAase-free tube and do one phenol extraction, followed by a chloroform:isoamylalcohol extraction. Add 1 vol. isopropanol to supernatant and chill on ice for 10 min. Pellet RNA by centrifugation (7000 rpm for 10 min at RT). Resolve pellet in 1 ml 4M LiCl by 10 to 15 min vortexing. Pellet RNA by 5 min centrifugation.

Second Purification. Resuspend pellet in 500 μl Resuspension buffer. Add 500 μl phenol and vortex. Add 250 μl chloroform:isoamylalcohol and vortex. Spin for 5 min. and transfer supernatant to fresh tube. Repeat chloroform:isoamylalcohol extraction until interface is clear. Transfer supernatant to fresh tube and add 1/10 vol 3M NaOAc, pH 5 and 600 μl isopropanol. Keep at −20 for 20 min or longer. Pellet RNA by 10 min centrifugation. Wash pellet once with 70% ethanol. Remove all remaining alcohol before resolving pellet with 15 to 20 μl DEPC-water. Determine quantity and quality by measuring the absorbance of a 1:200 dilution at 260 and 280 nm. 40 μg RNA/ml=1OD260

RNA from wild-type and the transgenic Arabidopsis-plants is isolated as described (Hosein, 2001, Plant Mol. Biol. Rep., 19, 65a-65e; Ruuska, S. A., Girke, T., Benning, C., & Ohlrogge, J. B., 2002, Plant Cell, 14, 1191-1206).

The mRNA is prepared from total RNA, using the Amersham Pharmacia Biotech mRNA purification kit, which utilizes oligo(dT)-cellulose columns.

Isolation of Poly-(A)+ RNA was isolated using Dyna BeadsR (Dynal, Oslo, Norway) following the instructions of the manufacturer's protocol. After determination of the concentration of the RNA or of the poly(A)+ RNA, the RNA was precipitated by addition of 1/10 volumes of 3 M sodium acetate pH 4.6 and 2 volumes of ethanol and stored at −70° C.

### Example 4

cDNA Library Construction. For cDNA library construction, first strand synthesis was achieved using Murine Leukemia Virus reverse transcriptase (Roche, Mannheim, Germany) and oligo-d(T)-primers, second strand synthesis by incubation with DNA polymerase I, Klenow enzyme and RNAseH digestion at 12° C. (2 h), 16° C. (1 h) and 22° C. (1 h). The reaction was stopped by incubation at 65° C. (10 min) and subsequently transferred to ice. Double stranded DNA molecules were blunted by T4-DNA-polymerase (Roche, Mannheim) at 37° C. (30 min). Nucleotides were removed by phenol/chloroform extraction and Sephadex G50 spin columns. EcoRI adapters (Pharmacia, Freiburg, Germany) were ligated to the cDNA ends by T4-DNA-ligase (Roche, 12° C., overnight) and phosphorylated by incubation with polynucleotide kinase (Roche, 37° C., 30 min). This mixture was subjected to separation on a low melting agarose gel. DNA molecules larger than 300 base pairs were eluted from the gel, phenol extracted, concentrated on Elutip-D-columns (Schleicher and Schuell, Dassel, Germany) and were ligated to vector arms and packed into lambda ZAPII phages or lambda ZAP-Express phages using the Gigapack Gold Kit (Stratagene, Amsterdam, Netherlands) using material and following the instructions of the manufacturer.

### Example 5

Northern-Hybridization. For RNA hybridization, 20 μg of total RNA or 1 μg of poly-(A)+ RNA is separated by gel electrophoresis in 1.25% agarose gels using formaldehyde as described in Amasino (1986, Anal. Biochem. 152:304), transferred by capillary attraction using 10×SSC to positively charged nylon membranes (Hybond N+, Amersham, Braunschweig), immobilized by UV light and pre-hybridized for 3 hours at 68° C. using hybridization buffer (10% dextran sulfate w/v, 1 M NaCl, 1% SDS, 100 μg/ml of herring sperm DNA). The labeling of the DNA probe with the Highprime DNA labeling kit (Roche, Mannheim, Germany) is carried out during the pre-hybridization using alpha-32P dCTP (Amersham, Braunschweig, Germany). Hybridization is carried out after addition of the labeled DNA probe in the same buffer at 68° C. overnight. The washing steps are carried out twice for 15 min using 2×SSC and twice for 30 min using 1×SSC, 1% SDS at 68° C. The exposure of the sealed filters is carried out at −70° C. for a period of 1 day to 14 days.

### Example 6

Plasmids for Plant Transformation. For plant transformation binary vectors such as pBinAR can be used (Höfgen & Willmitzer 1990, Plant Sci. 66:221-230). Construction of the binary vectors can be performed by ligation of the cDNA in sense or antisense orientation into the T-DNA. 5′ to the cDNA a plant promoter activates transcription of the cDNA. A polyadenylation sequence is located 3′ to the cDNA. Tissue-specific expression can be achieved by using a tissue specific promoter. For example, seed-specific expression can be achieved by cloning the napin or LeB4 or USP promoter 5′ to the cDNA. Also any other seed specific promoter element can be used. For constitutive expression within the whole plant the CaMV 35S promoter can be used. The expressed protein can be targeted to a cellular compartment using a signal peptide, for example for plastids, mitochondria, or endoplasmic reticulum (Kermode 1996, Crit. Rev. Plant Sci. 15:285-423). The signal peptide is cloned 5′ in frame to the cDNA to achieve subcellular localization of the fusion protein.

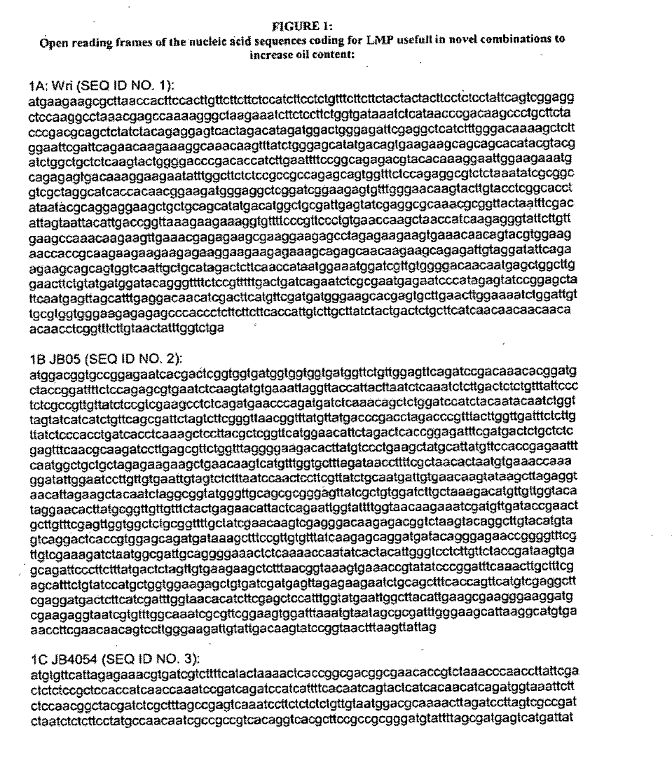

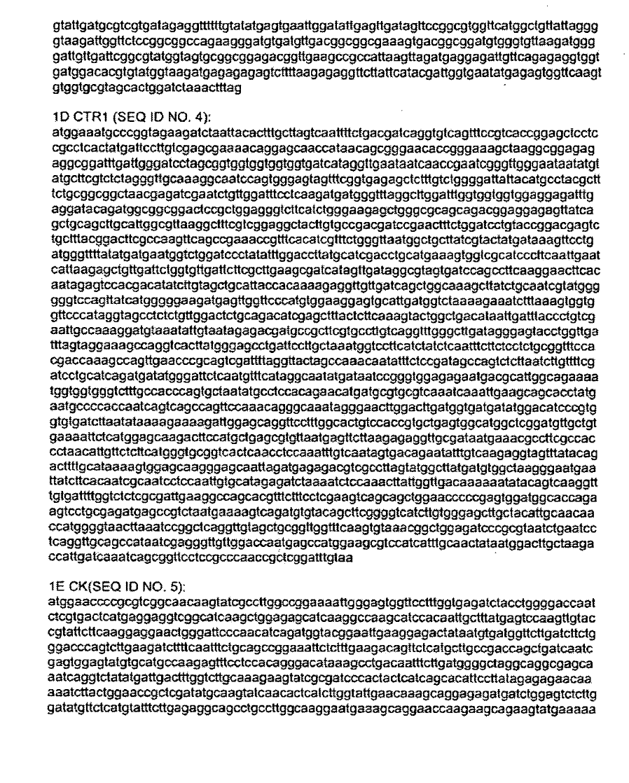

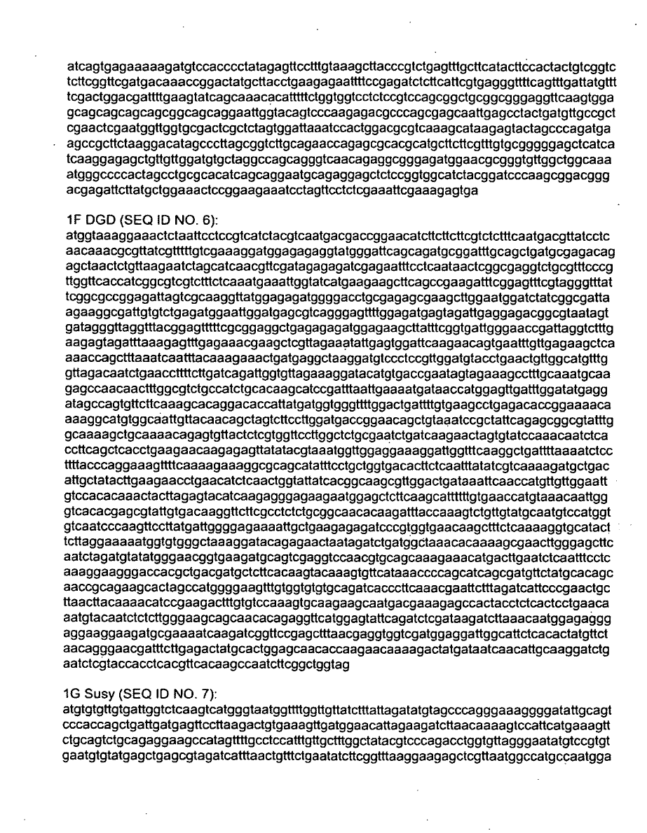

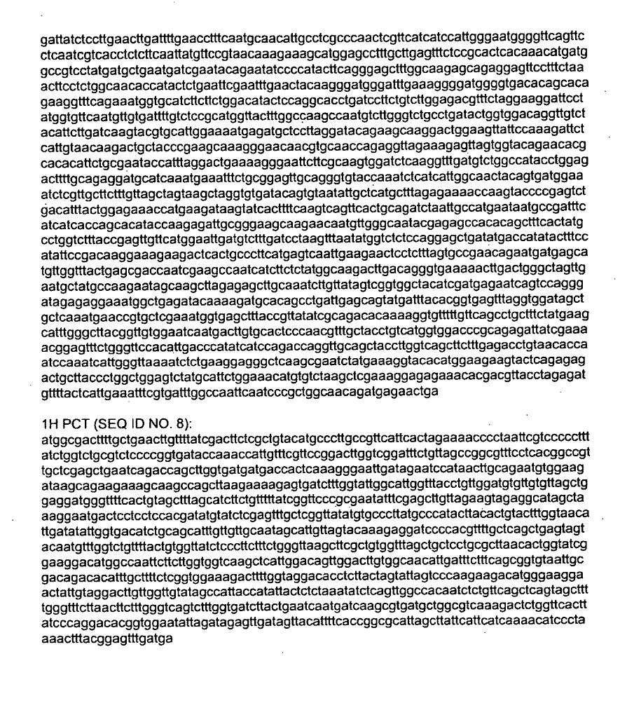

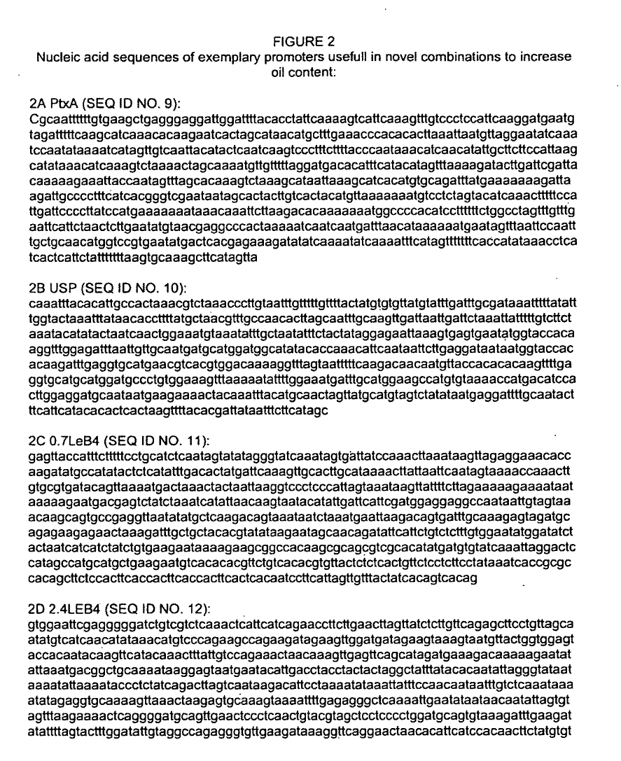

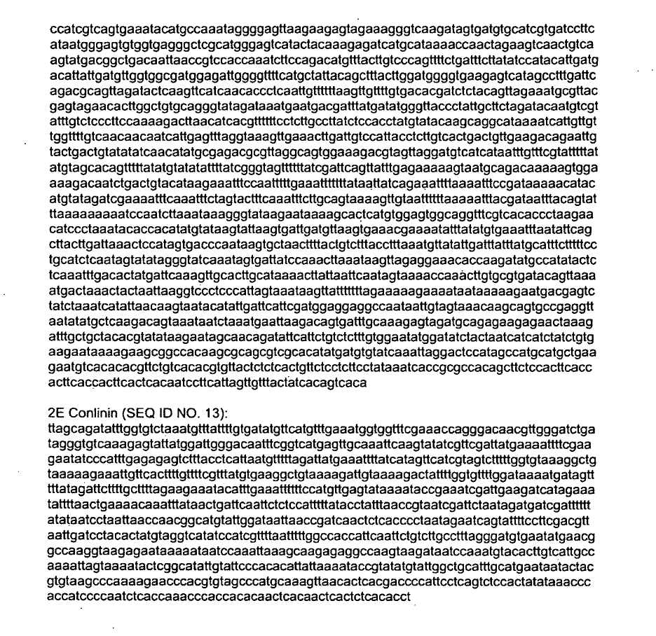

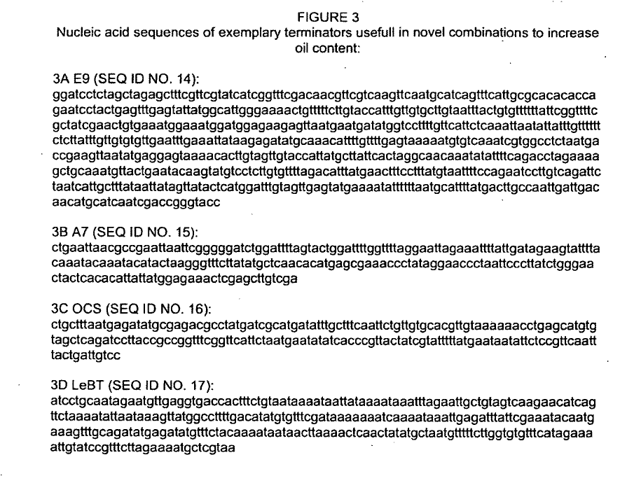

Further examples for plant binary vectors are the pSUN300 or pSUN2-GW vectors, into which the combination of LMP genes are cloned. These binary vectors contain an antibiotic resistance gene driven under the control of the NOS promoter and combinations (see Table 9 of FIG. 8) containing promoters as listed in FIG. 2, LMP genes as shown in FIG. 1 and terminators in FIG. 3 Partial or full-length LMP cDNA are cloned into the multiple cloning site of the pEntry vector in sense or antisense orientation behind a seed-specific promoters or constitutive promoter (see FIG. 2) in the combinations shown in Table 9 of FIG. 8 using standard cloning procedures using restriction enzymes such as ASCI, PACI, NotP and StuI. Two or more pEntry vectors containing different LMPs are then combined with a pSUN destination vector to form a binary vector containing the combinations as listed in Table 9 of FIG. 8 by the use of the GATEWAY technology (Invitrogen, http://www.invitrogen.com) following the manufacturer's instructions. The recombinant vector containing the combination of interest is transformed into Top10 cells (invitrogen) using standard conditions. Transformed cells are selected for on LB agar containing 50 μg/ml kanamycin grown overnight at 37° C. Plasmid DNA is extracted using the QIAprep Spin Miniprep Kit (Qiagen) following manufacturer's instructions. Analysis of subsequent clones and restriction mapping is performed according to standard molecular biology techniques (Sambrook et al. 1989, Molecular Cloning, A Laboratory Manual. 2nd Edition. Cold Spring Harbor Laboratory Press. Cold Spring Harbor, N.Y.).

### Example 7

Agrobacterium Mediated Plant Transformation. Agrobacterium mediated plant transformation with the combination of LMP nucleic acids described herein can be performed using standard transformation and regeneration techniques (Gelvin, Stanton B. & Schilperoort R. A, Plant Molecular Biology Manual, 2nd ed. Kluwer Academic Publ., Dordrecht 1995 in Sect., Ringbuc Zentrale Signatur:BT11-P; Glick, Bernard R. and Thompson, John E. Methods in Plant Molecular Biology and Biotechnology, S. 360, CRC Press, Boca Raton 1993). For example, Agrobacterium mediated transformation can be performed using the GV3 (pMP90) (Koncz & Schell, 1986, Mol. Gen. Genet. 204:383-396) or LBA4404 (Clontech) Agrobacterium tumefaciens strain.

Arabidopsis thaliana can be grown and transformed according to standard conditions (Bechtold 1993, Acad. Sci. Paris. 316:1194-1199; Bent et al. 1994, Science 265:1856-1860). Additionally, rapeseed can be transformed with the combination of LMP nucleic acids of the present invention via cotyledon or hypocotyl transformation (Moloney et al. 1989, Plant Cell Report 8:238-242; De Block et al. 1989, Plant Physiol. 91:694-701). Use of antibiotic for Agrobacterium and plant selection depends on the binary vector and the Agrobacterium strain used for transformation. Rapeseed selection is normally performed using a selectable plant marker. Additionally, Agrobacterium mediated gene transfer to flax can be performed using, for example, a technique described by Mlynarova et al. (1994, Plant Cell Report 13:282-285).

The LMPs in the combinations described in this invention can be expressed either under the seed specific USP (unknown seed protein) promoter (Baeumlein et al. 1991, Mol. Gen. Genetics 225:459-67), the PtxA promoter (the promoter of the Pisum sativum PtxA gene), which is a promoter active in virtually all plant tissues or the superpromoter, which is a constitutive promoter (Stanton B. Gelvin, U.S. Pat. No. 5,428,147 and U.S. Pat. No. 5,217,903) or other seed-specific promoters like the legumin B4 promoter (LeB4; Baeumlein et al. 1992, Plant J. 2:233-239), as well as promoters conferring seed-specific expression in monocot plants like maize, barley, wheat, rye, rice, etc. were used.

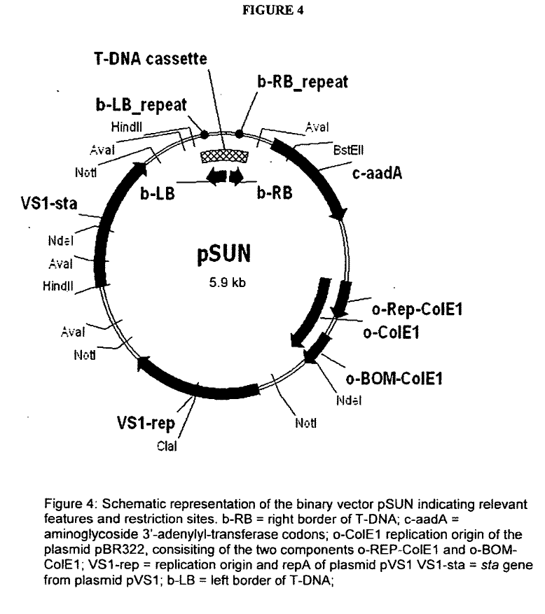

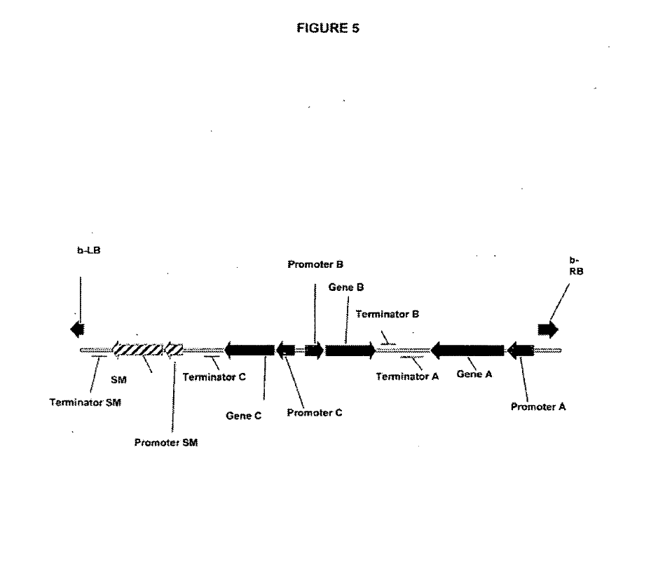

The nptII gene was used as a selectable marker in these constructs. FIGS. 4 and 5 show the setup of the binary vectors containing the combinations of LMPs.

Transformation of soybean can be performed using, for example, a technique described in EP 0424 047, U.S. Pat. No. 5,322,783 (Pioneer Hi-Bred International) or in EP 0397 687, U.S. Pat. No. 5,376,543 or U.S. Pat. No. 5,169,770 (University Toledo), or by any of a number of other transformation procedures known in the art. Soybean seeds are surface sterilized with 70% ethanol for 4 minutes at room temperature with continuous shaking, followed by 20% (v/v) CLOROX supplemented with 0.05% (v/v) TWEEN for 20 minutes with continuous shaking. Then the seeds are rinsed 4 times with distilled water and placed on moistened sterile filter paper in a Petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile Petri dish and air-dried to a moisture content less than 20% (fresh weight) in a sealed Petri dish until further use.

The method of plant transformation is also applicable to Brassica napus and other crops. In particular, seeds of canola are surface sterilized with 70% ethanol for 4 minutes at room temperature with continuous shaking, followed by 20% (v/v) CLOROX supplemented with 0.05% (v/v) TWEEN for 20 minutes, at room temperature with continuous shaking. Then, the seeds are rinsed four times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use.

Agrobacterium tumefaciens culture is prepared from a single colony in LB solid medium plus appropriate antibiotics (e.g. 100 mg/l streptomycin, 50 mg/l kanamycin) followed by growth of the single colony in liquid LB medium to an optical density at 600 nm of 0.8. Then, the bacteria culture is pelleted at 7000 rpm for 7 minutes at room temperature, and resuspended in MS (Murashige & Skoog 1962, Physiol. Plant. 15:473-497) medium supplemented with 100 mM acetosyringone. Bacteria cultures are incubated in this pre-induction medium for 2 hours at room temperature before use. The axis of soybean zygotic seed embryos at approximately 44% moisture content are imbibed for 2 hours at room temperature with the pre-induced Agrobacterium suspension culture. (The imbibition of dry embryos with a culture of Agrobacterium is also applicable to maize embryo axes). The embryos are removed from the imbibition culture and are transferred to Petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/l carbenicillin or 300 mg/l cefotaxime to kill the agrobacteria. The liquid medium is used to moisten the sterile filter paper. The embryos are incubated during 4 weeks at 25° C., under 440 μmol m-2s-1 and 12 hours photoperiod. Once the seedlings have produced roots, they are transferred to sterile metromix soil. The medium of the in vitro plants is washed off before transferring the plants to soil. The plants are kept under a plastic cover for 1 week to favor the acclimatization process. Then the plants are transferred to a growth room where they are incubated at 25° C., under 440 μmol m-2s-1 light intensity and 12-hour photoperiod for about 80 days.

Samples of the primary transgenic plants (TO) are analyzed by PCR to confirm the presence of T-DNA. These results are confirmed by Southern hybridization wherein DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labeled probe by PCR as recommended by the manufacturer.

### Example 7

In vivo Mutagenesis. In vivo mutagenesis of microorganisms can be performed by incorporation and passage of the plasmid (or other vector) DNA through E. coli or other microorganisms (e.g. Bacillus spp. or yeasts such as Sacchromyces) that are impaired in their capabilities to maintain the integrity of their genetic information. Typical mutator strains have mutations in the genes for the DNA repair system (e.g., mutHLS, mutD, mutT, etc.; for reference, see Rupp W. D. 1996, DNA repair mechanisms, in: Escherichia coli and Salmonella, p. 2277-2294, ASM: Washington). Such strains are well known to those skilled in the art. The use of such strains is illustrated, for example, in Greener and Callahan 1994, Strategies 7:32-34. Transfer of mutated DNA molecules into plants is preferably done after selection and testing in microorganisms. Transgenic plants are generated according to various examples within the exemplification of this document.

### Example 8

Assessment of the mRNA Expression and Activity of a Recombinant Gene Product in the Transformed Organism. The activity of a recombinant gene product in the transformed host organism can be measured on the transcriptional or/and on the translational level. A useful method to ascertain the level of transcription of the gene (an indicator of the amount of mRNA available for translation to the gene product) is to perform a Northern blot (for reference see, for example, Ausubel et al. 1988, Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information at least partially demonstrates the degree of transcription of the transformed gene. Total cellular RNA can be prepared from plant cells, tissues or organs by several methods, all well-known in the art, such as that described in Bormann et al. (1992, Mol. Microbiol. 6:317-326).

To assess the presence or relative quantity of protein translated from this mRNA, standard techniques, such as a Western blot, may be employed (see, for example, Ausubel et al. 1988, Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or colorimetric label, which may be readily detected. The presence and quantity of label observed indicates the presence and quantity of the desired mutant protein present in the cell.

The activity of LMPs that bind to DNA can be measured by several well-established methods, such as DNA band-shift assays (also called gel retardation assays). The effect of such LMP on the expression of other molecules can be measured using reporter gene assays (such as that described in Kolmar H. et al. 1995, EMBO J. 14:3895-3904 and references cited therein). Reporter gene test systems are well known and established for applications in both prokaryotic and eukaryotic cells, using enzymes, such as beta-galactosidase, green fluorescent protein, and several others.

The determination of activity of lipid metabolism membrane-transport proteins can be performed according to techniques such as those described in Gennis R. B. (1989 Pores, Channels and Transporters, in Biomembranes, Molecular Structure and Function, Springer: Heidelberg, pp. 85-137, 199-234 and 270-322).

### Example 8

In vitro Analysis of the activity of LMPS expressed in combinations in Transgenic Plants. The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications, and examples for the determination of many enzyme activities may be found, for example, in the following references: Dixon, M. & Webb, E. C. 1979, Enzymes. Longmans: London; Fersht, (1985) Enzyme Structure and Mechanism. Freeman: New York; Walsh (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3rd ed. Academic Press: New York; Bisswanger, H., (1994) Enzymkinetik, 2nd ed. VCH: Weinheim (ISBN 3527300325); Bergmeyer, H. U., Bergmeyer, J., Graβl, M., eds. (1983-1986) Methods of Enzymatic Analysis, 3rd ed., vol. I-XII, Verlag Chemie: Weinheim; and Ullmann's Encyclopedia of Industrial Chemistry (1987) vol. A9, Enzymes. VCH: Weinheim, p. 352-363.

### Example 9

Analysis of the Impact of Combinations of Recombinant Proteins on the Production of a Desired Seed Storage Compound. Seeds from transformed Arabidopsis thaliana plants were analyzed by gas chromatography (GC) for total oil content and fatty acid profile. GC analysis reveals that Arabidopsis plants transformed with a construct containing a combination of LMPs as described herein.

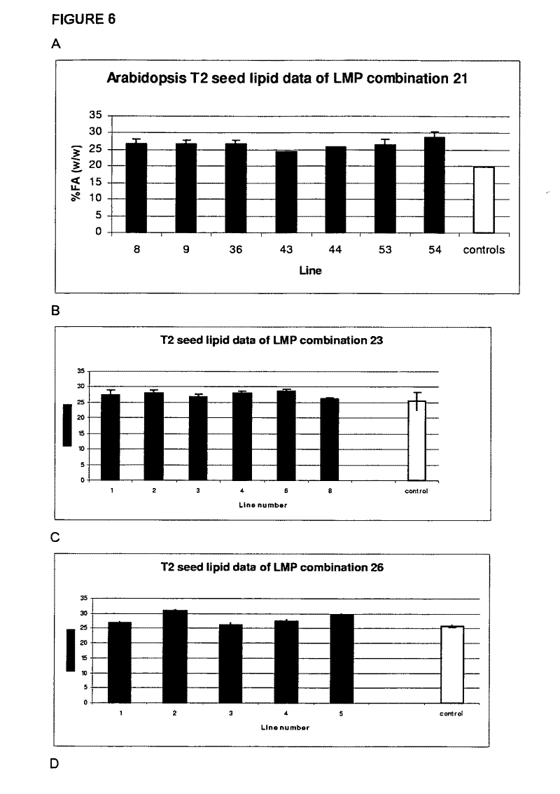

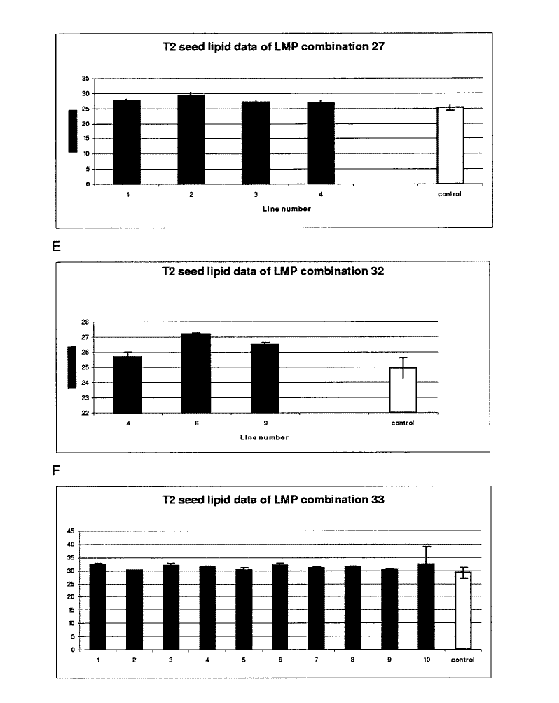

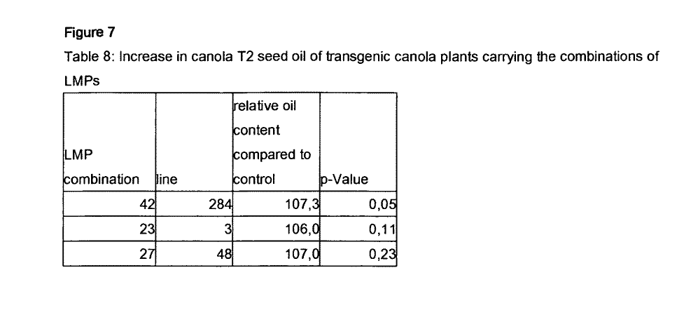

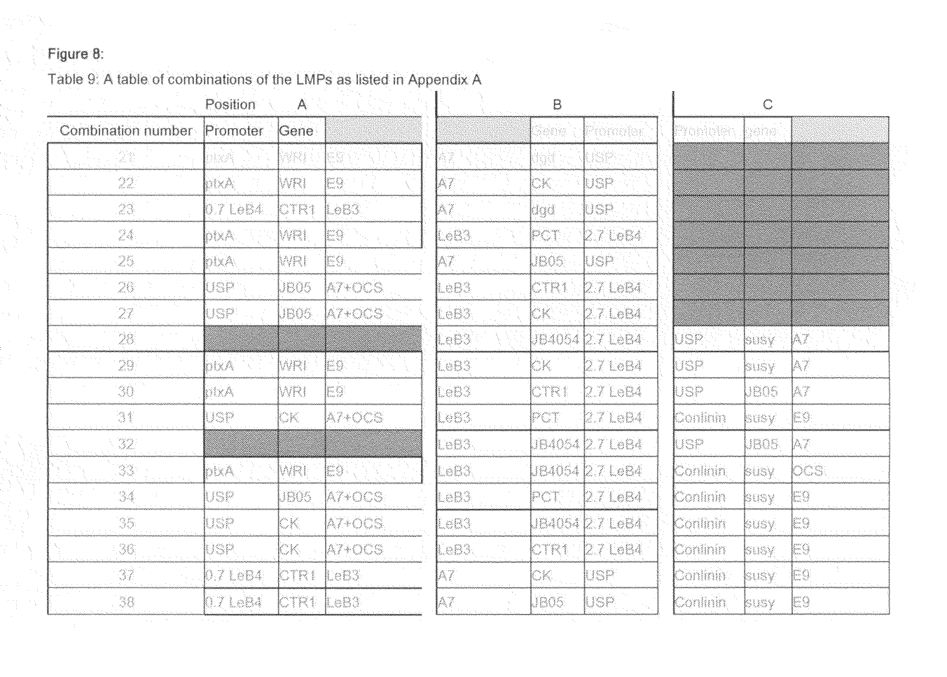

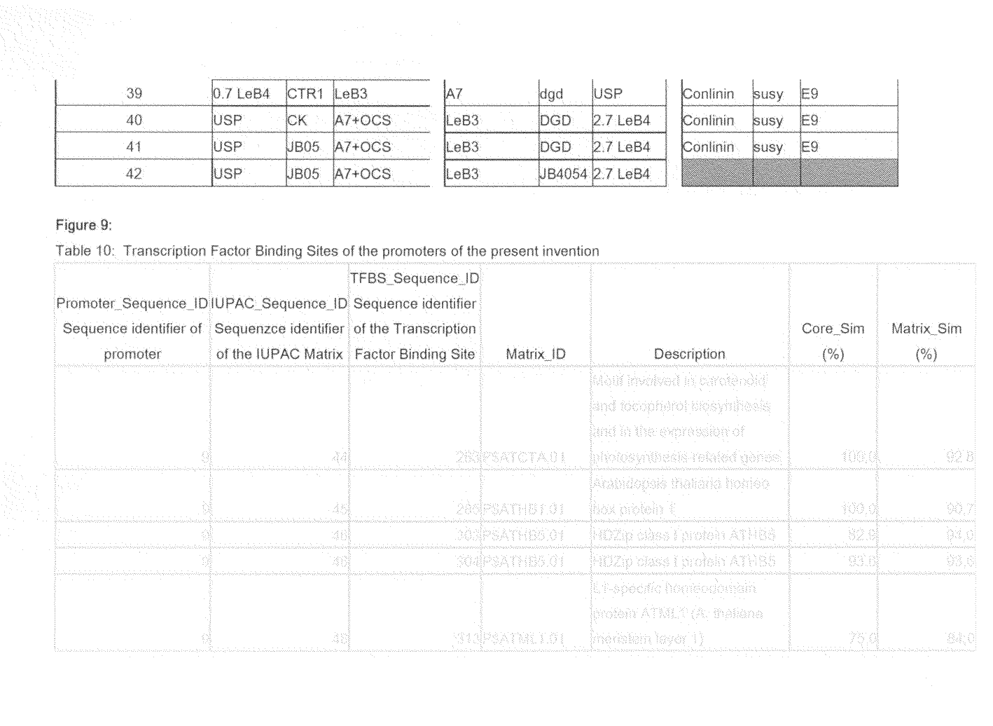

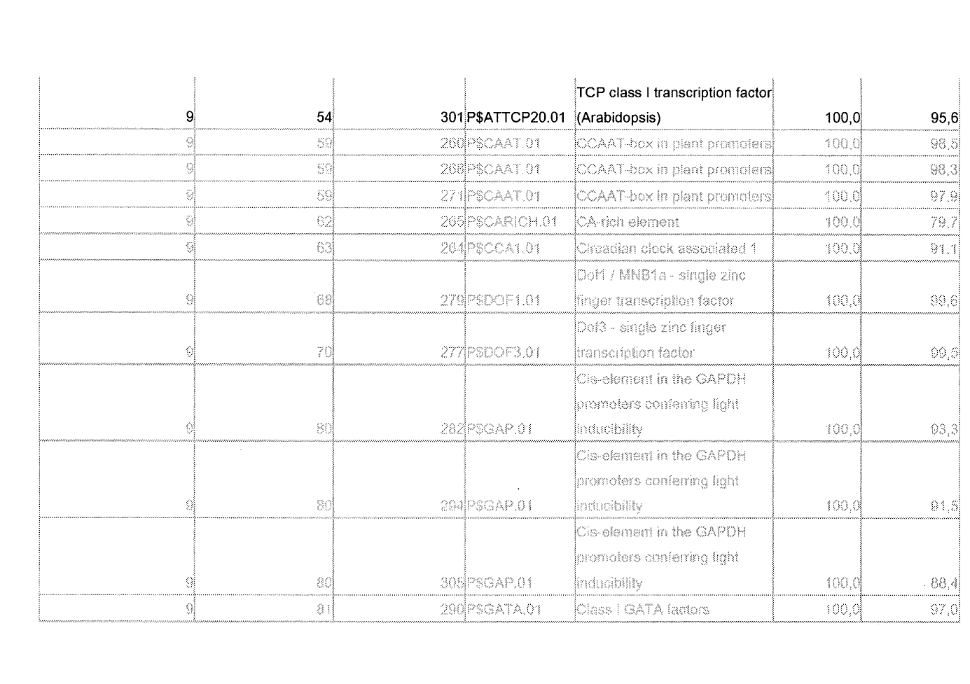

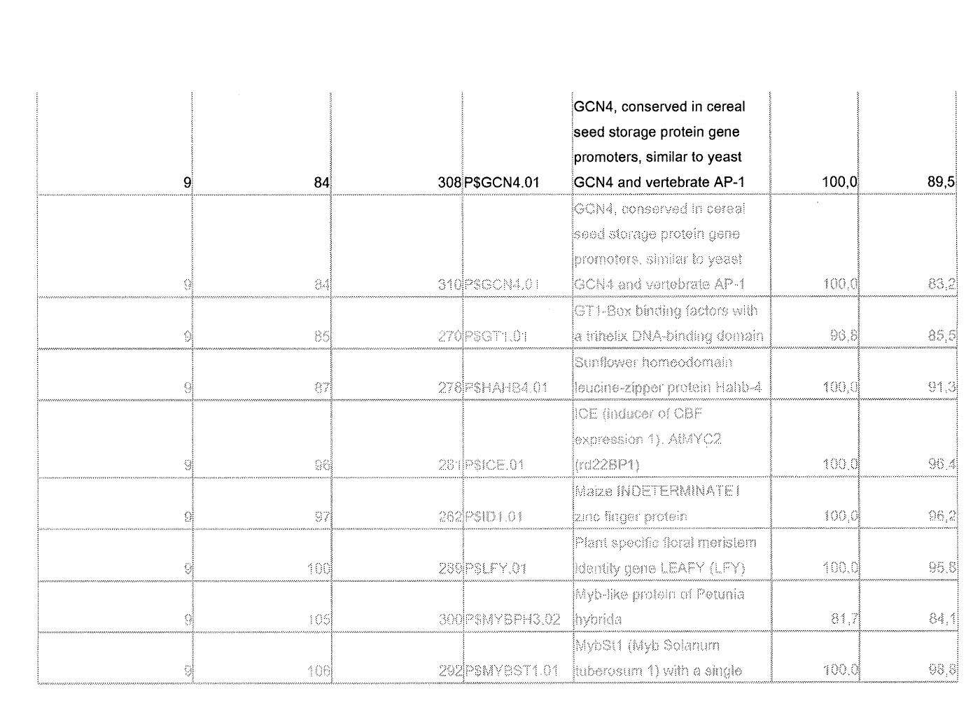

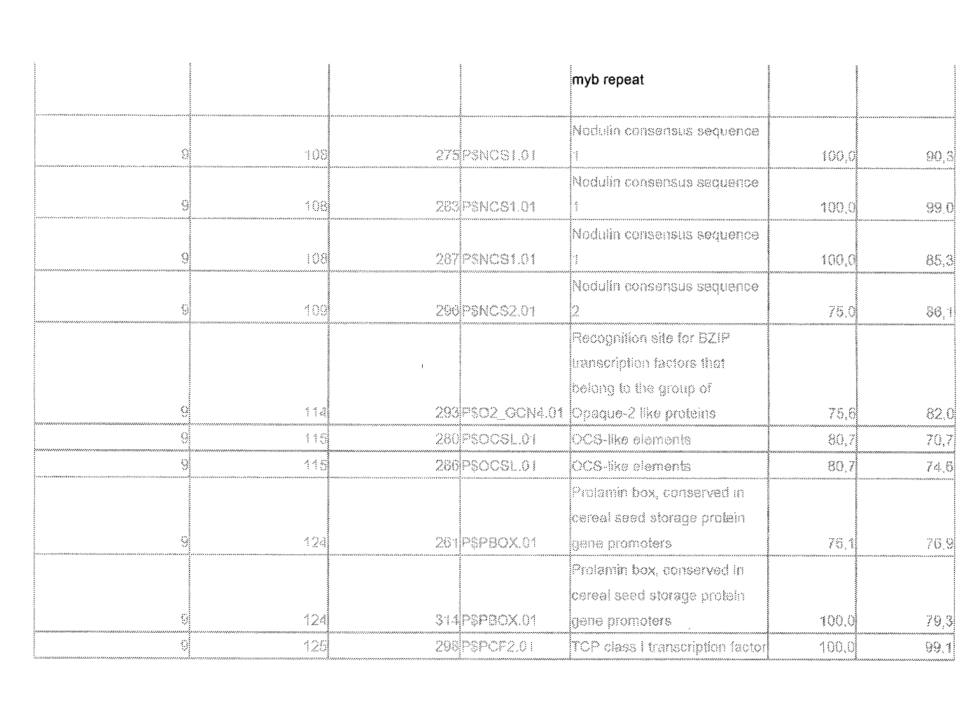

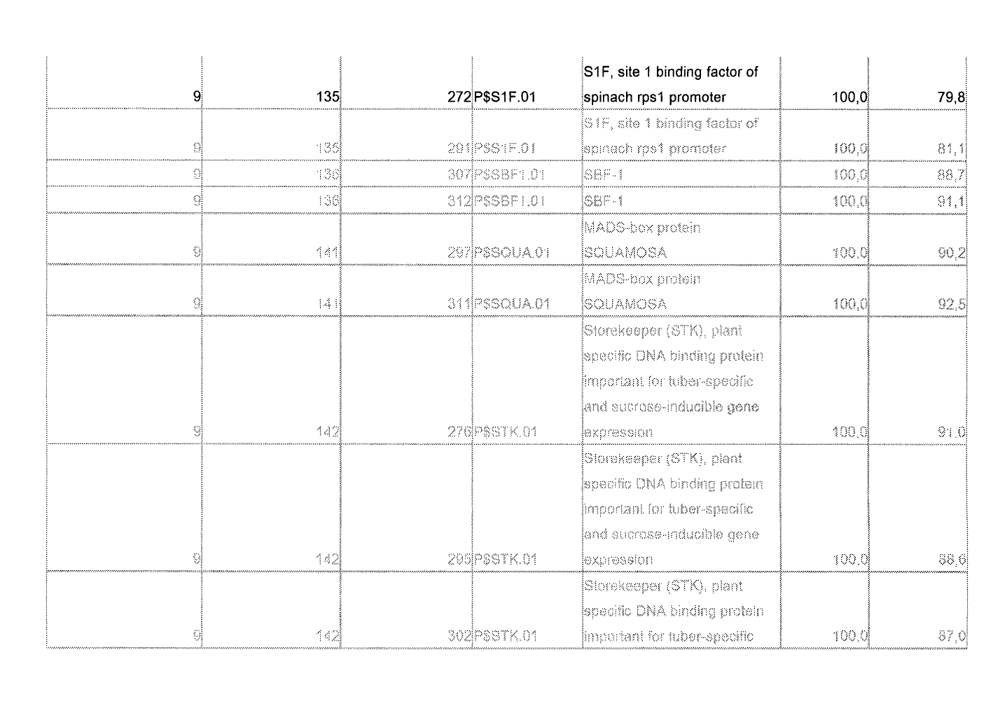

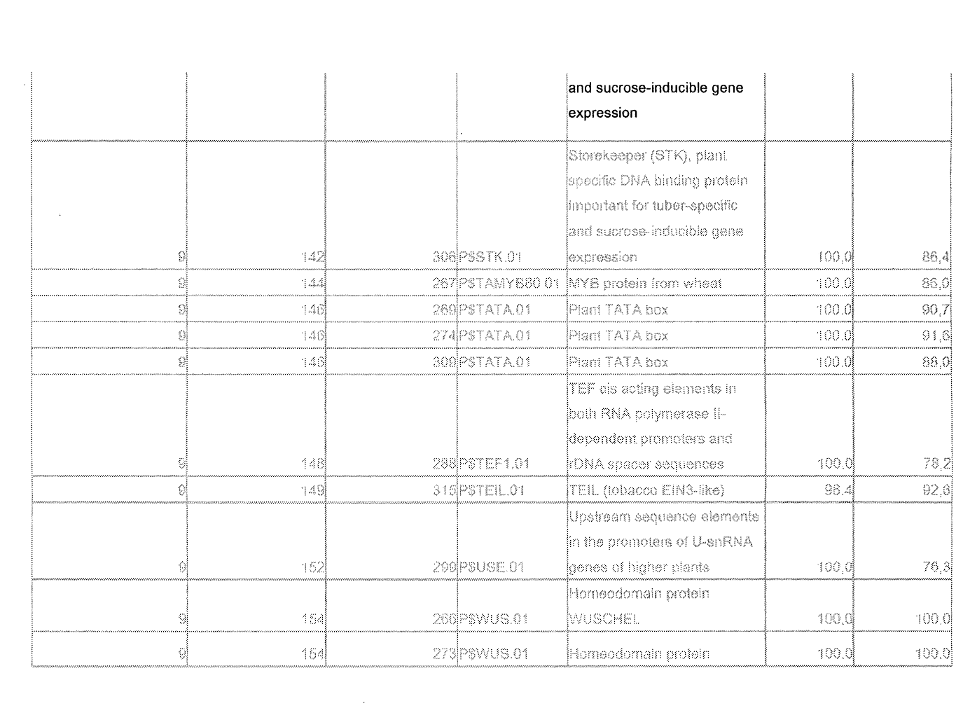

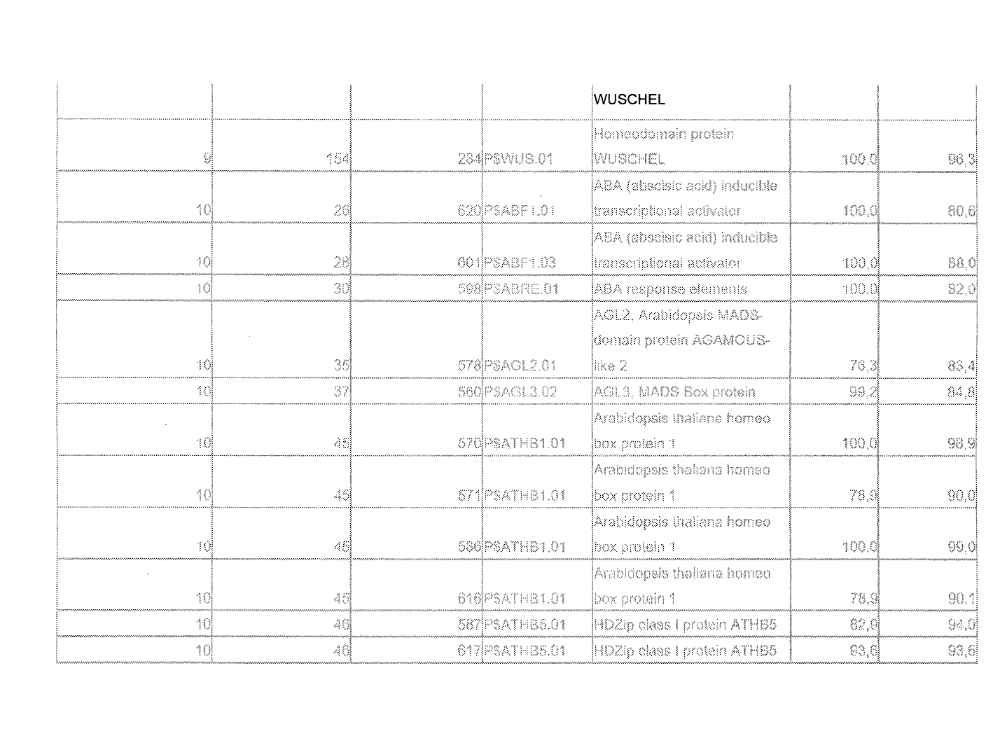

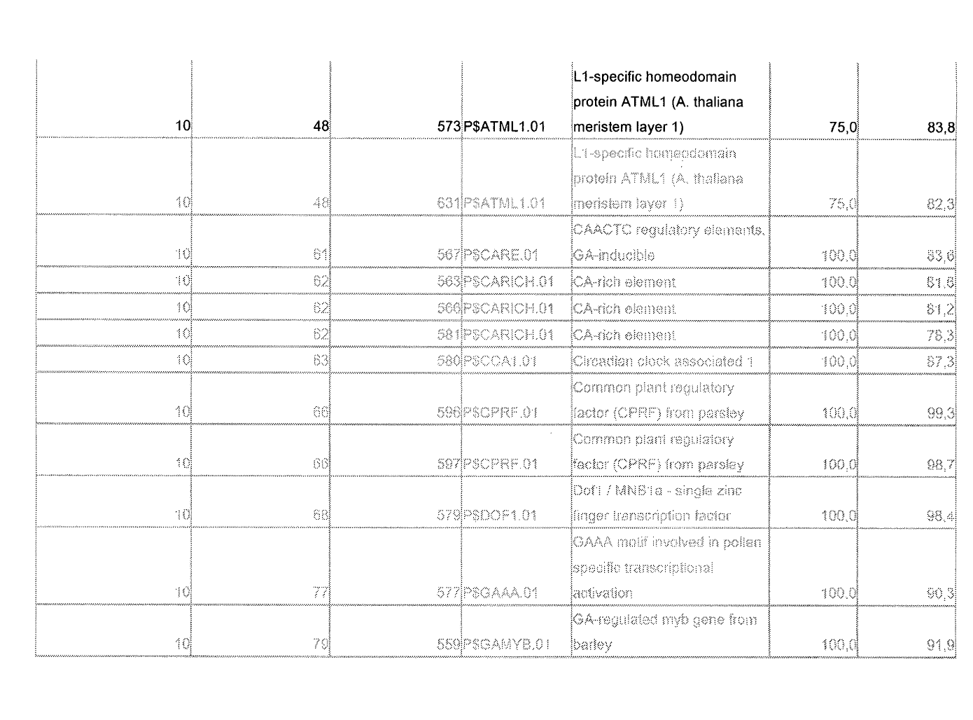

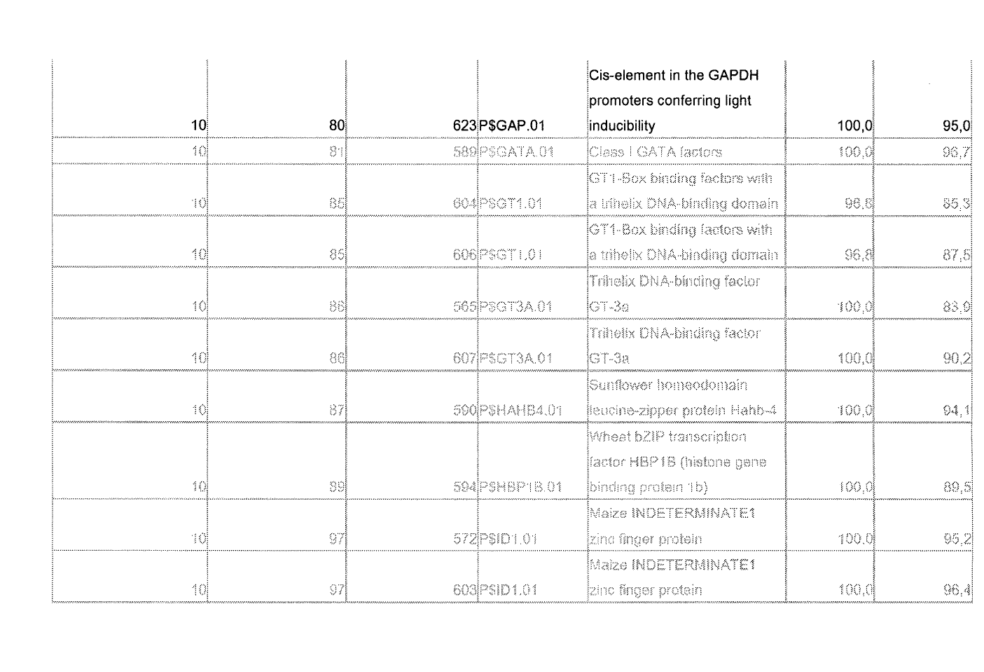

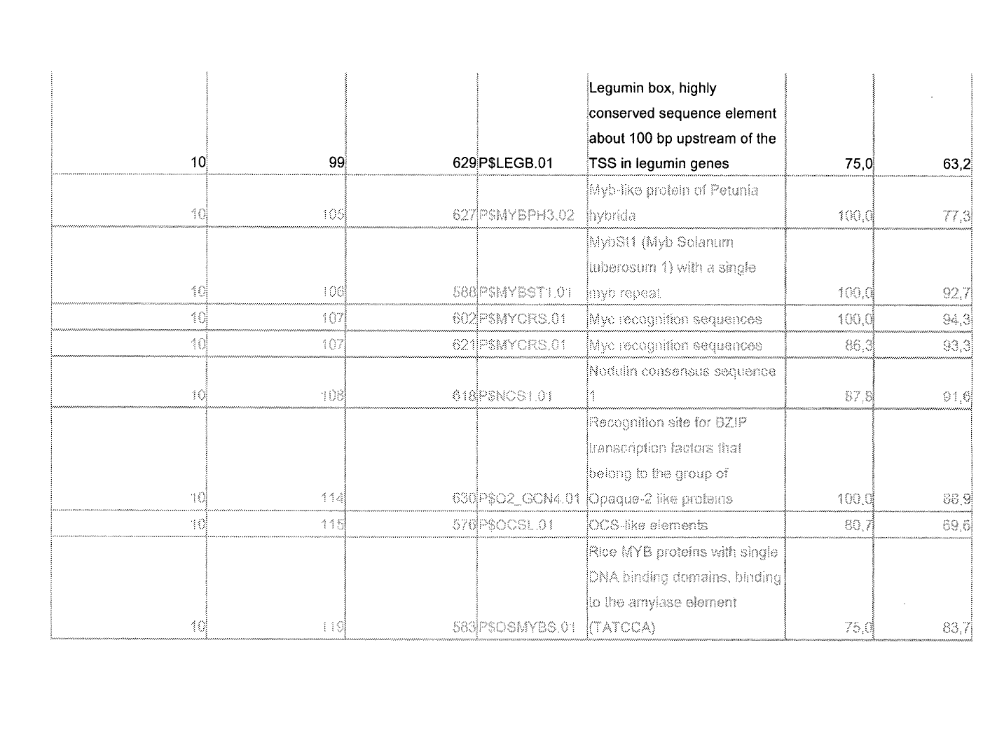

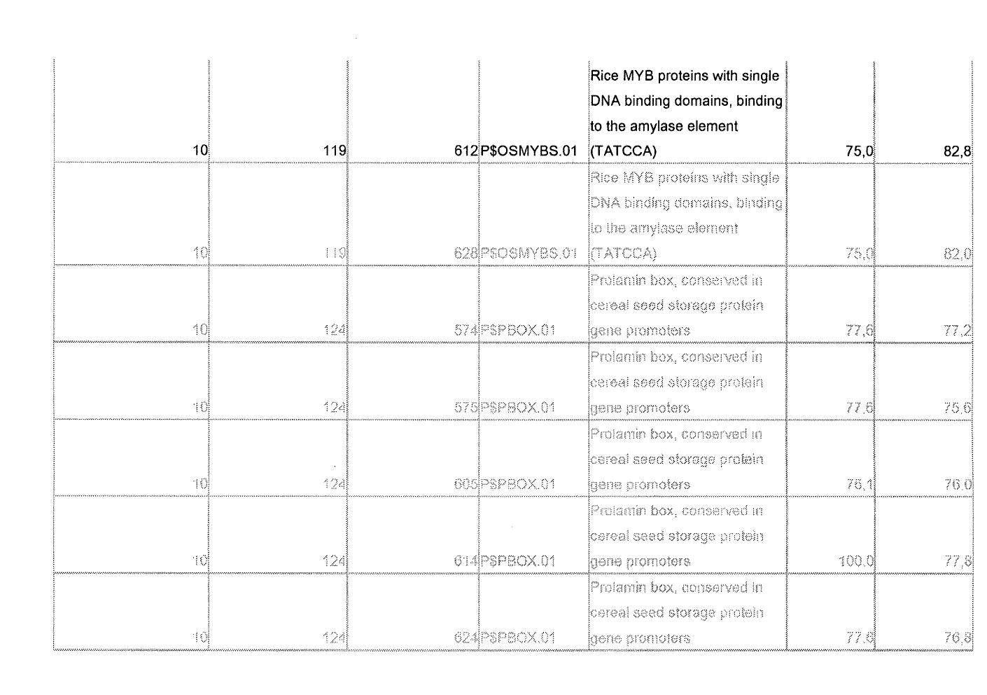

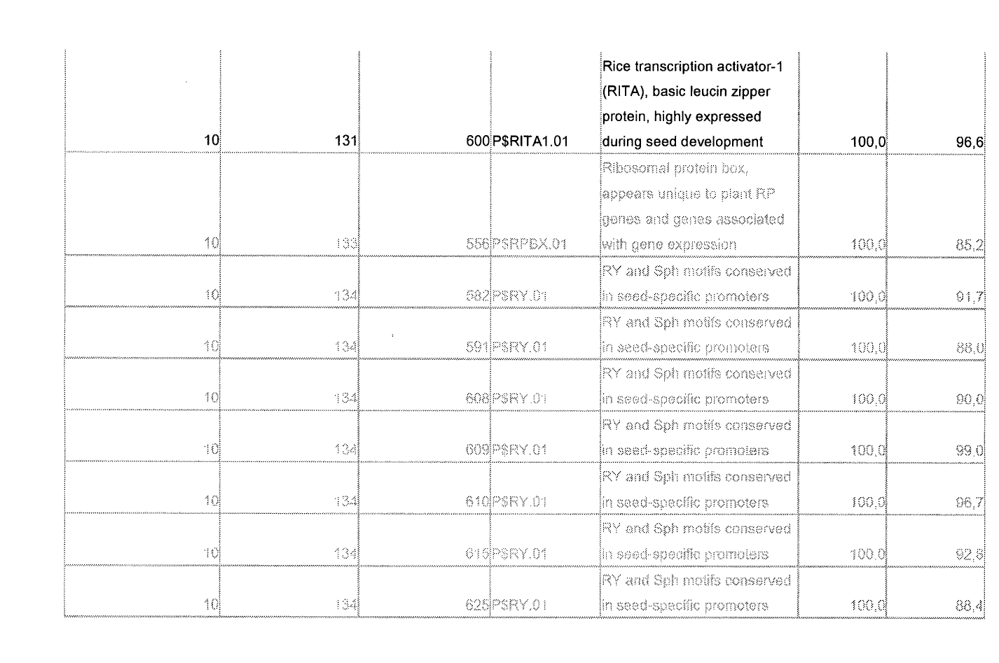

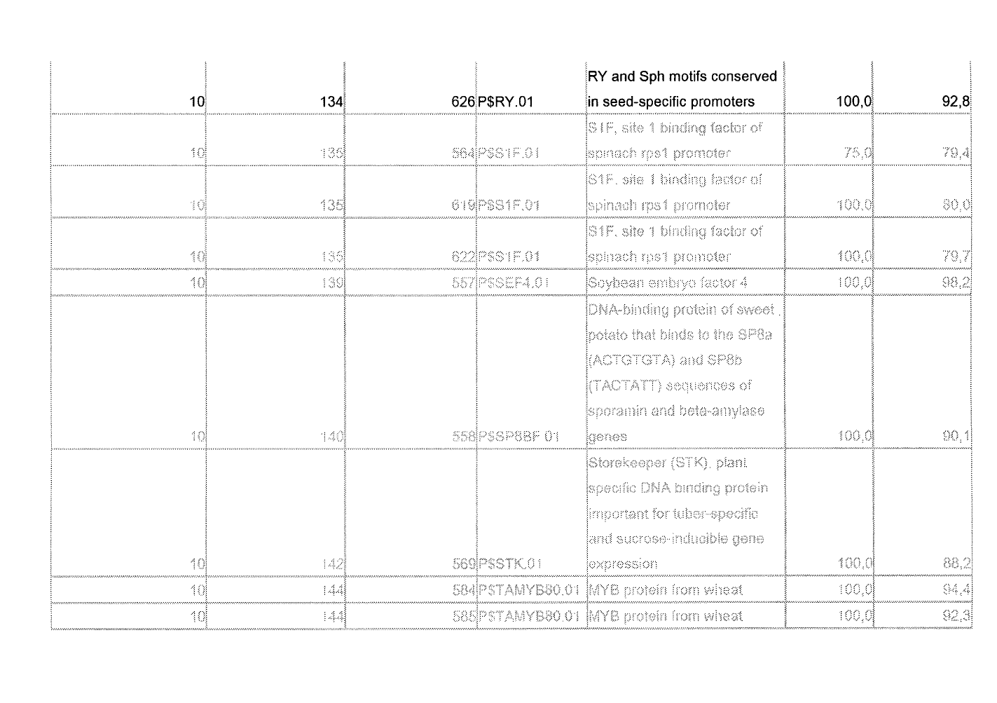

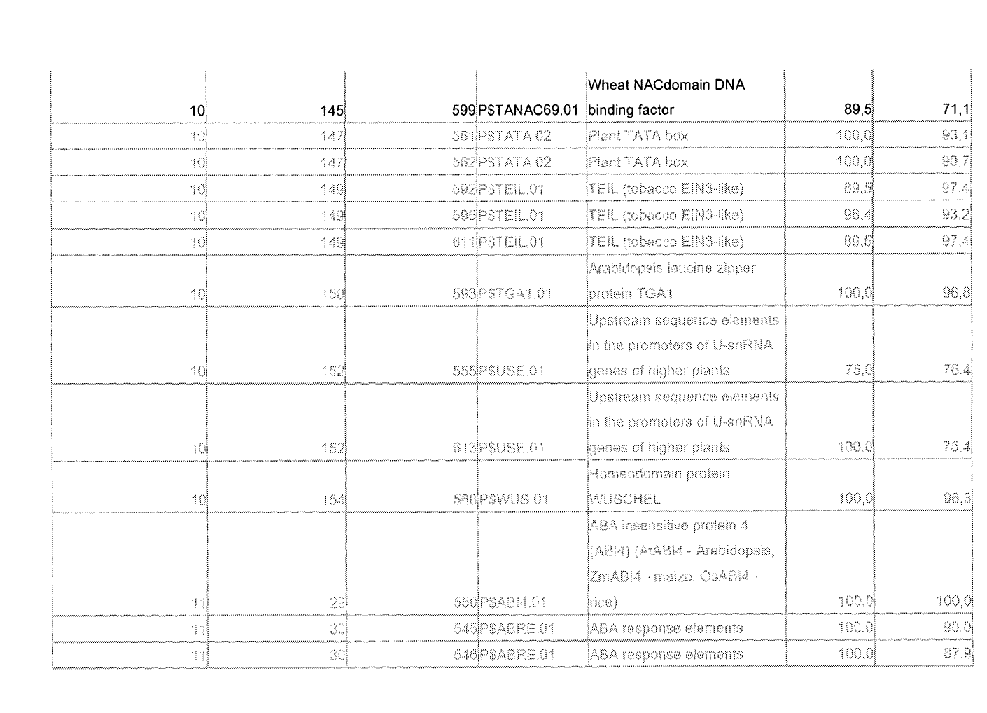

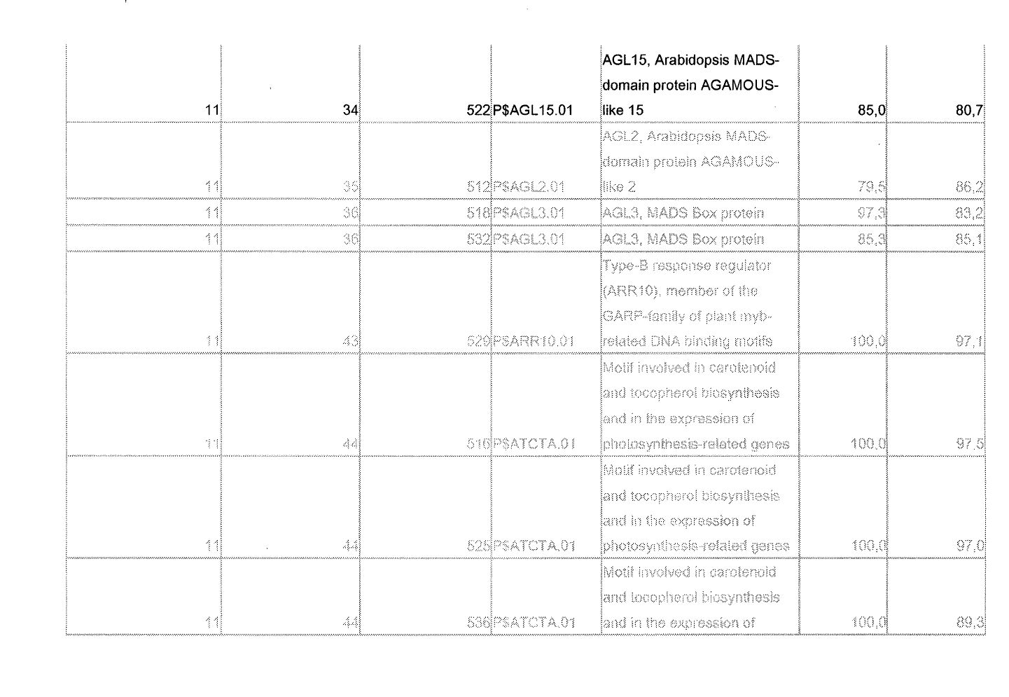

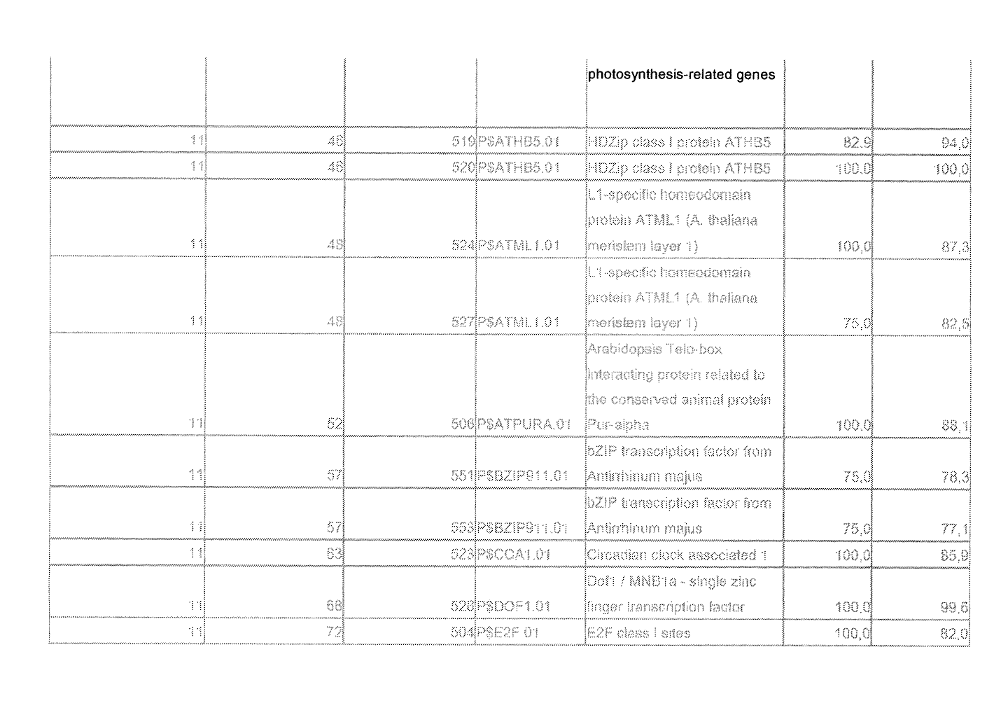

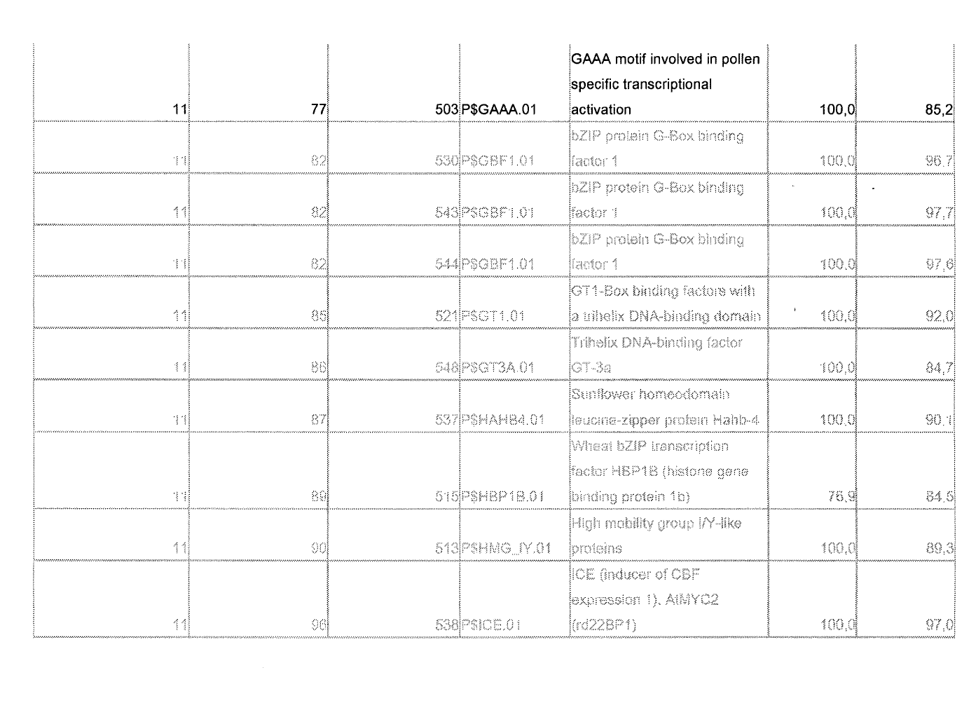

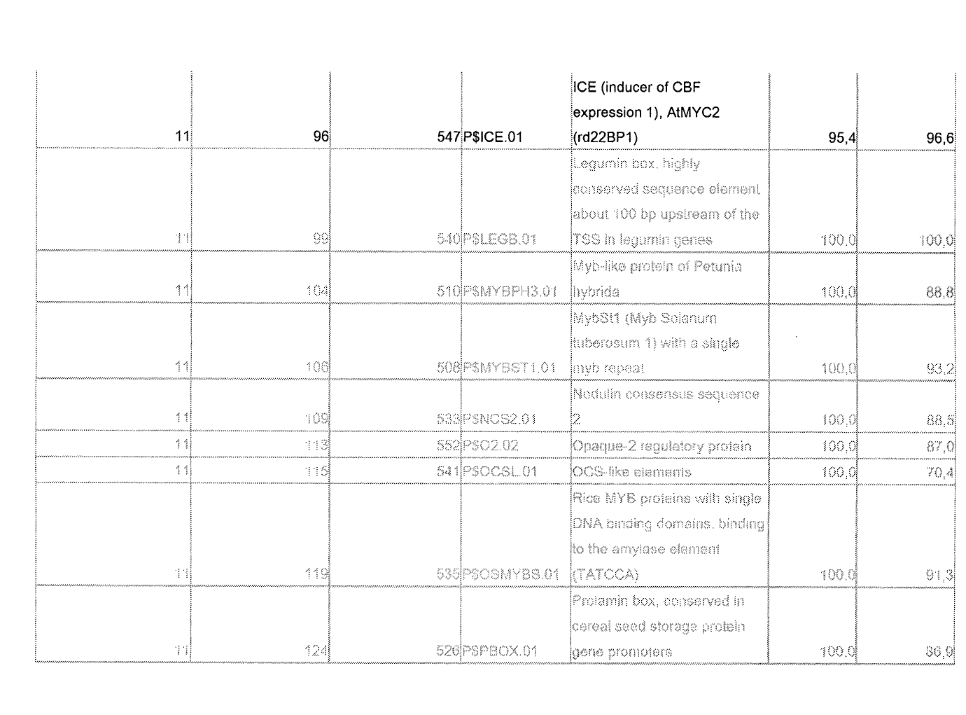

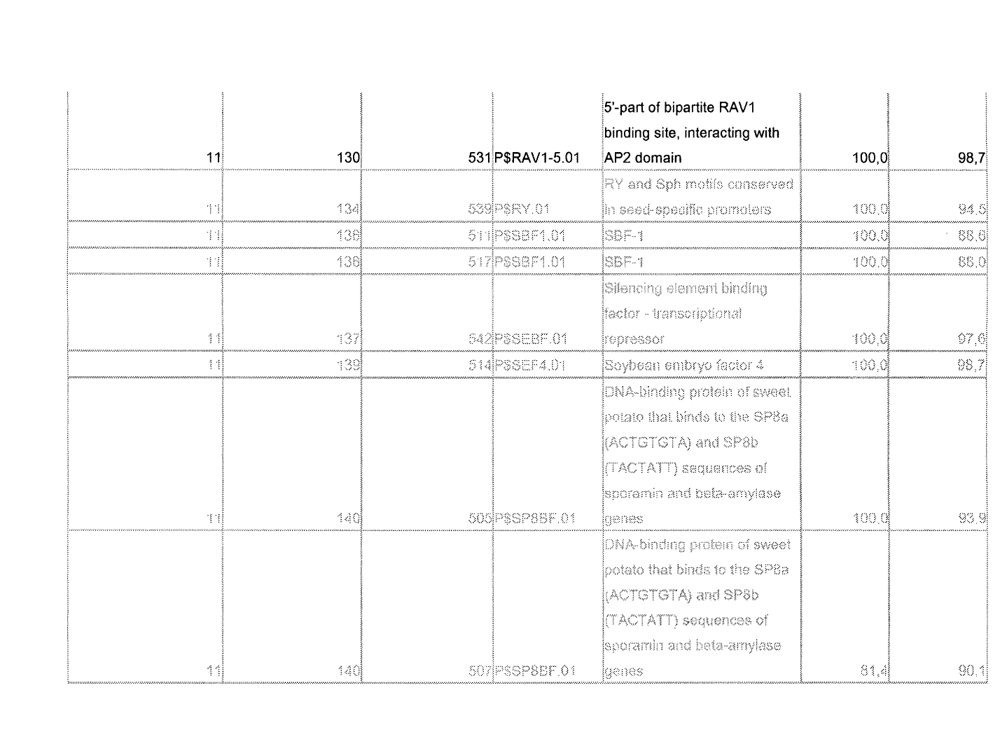

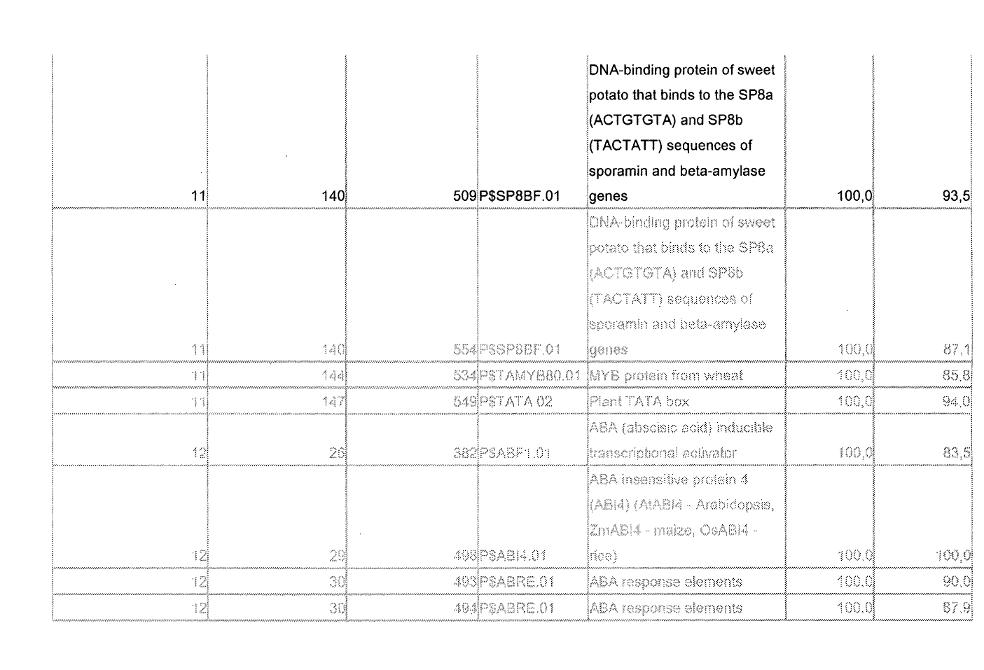

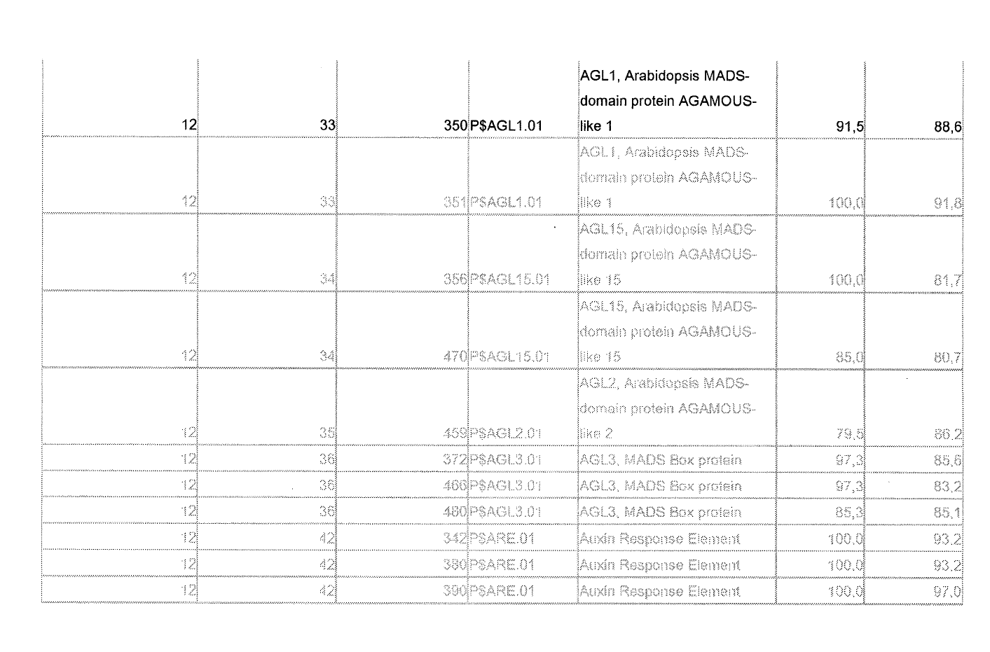

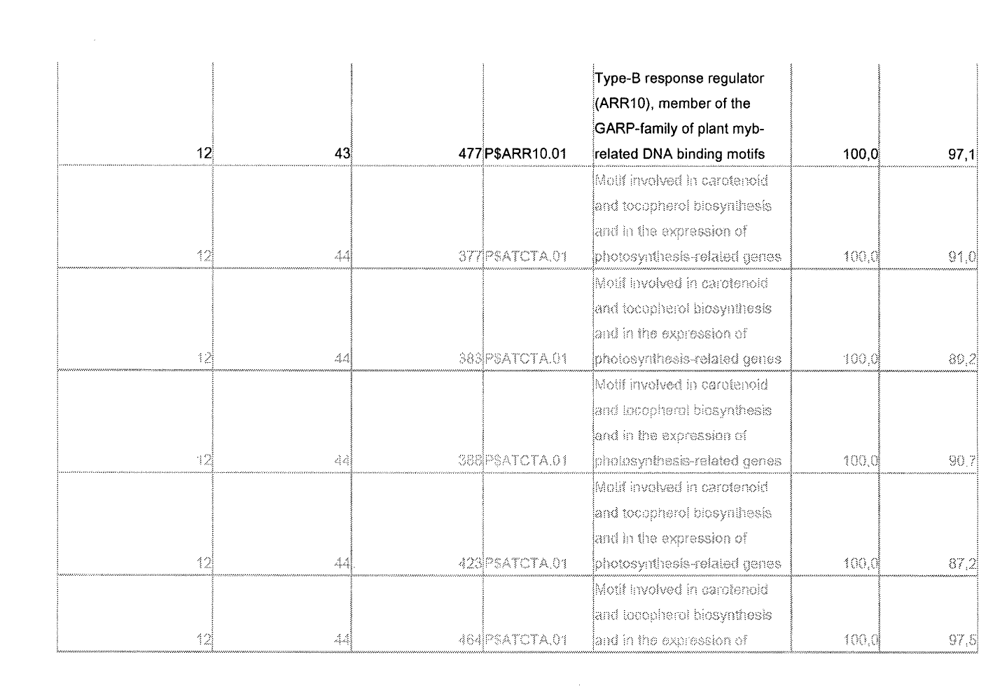

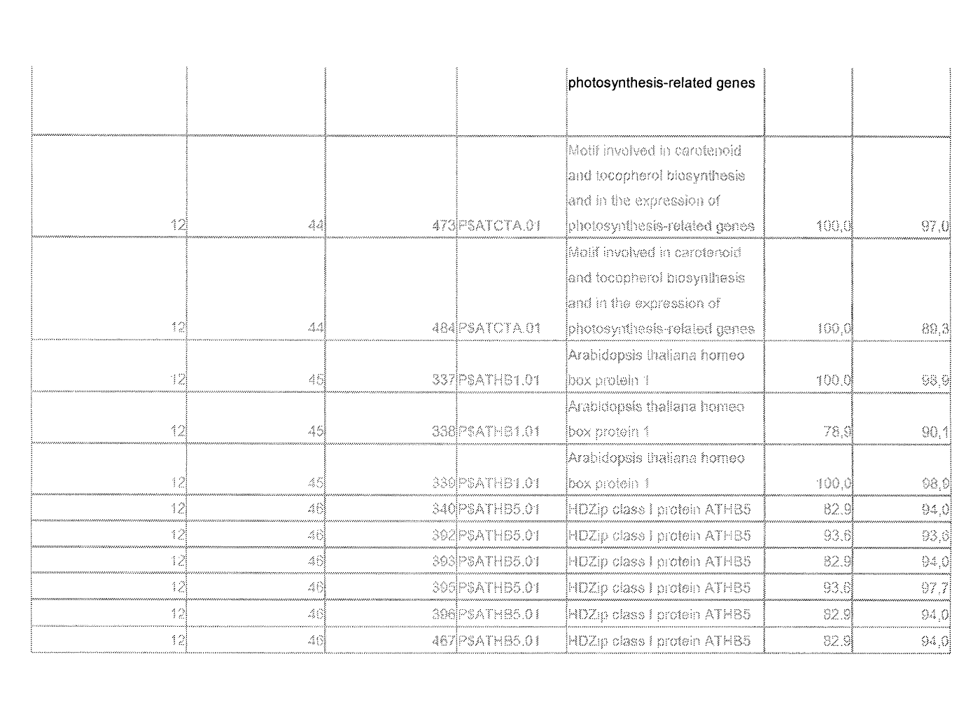

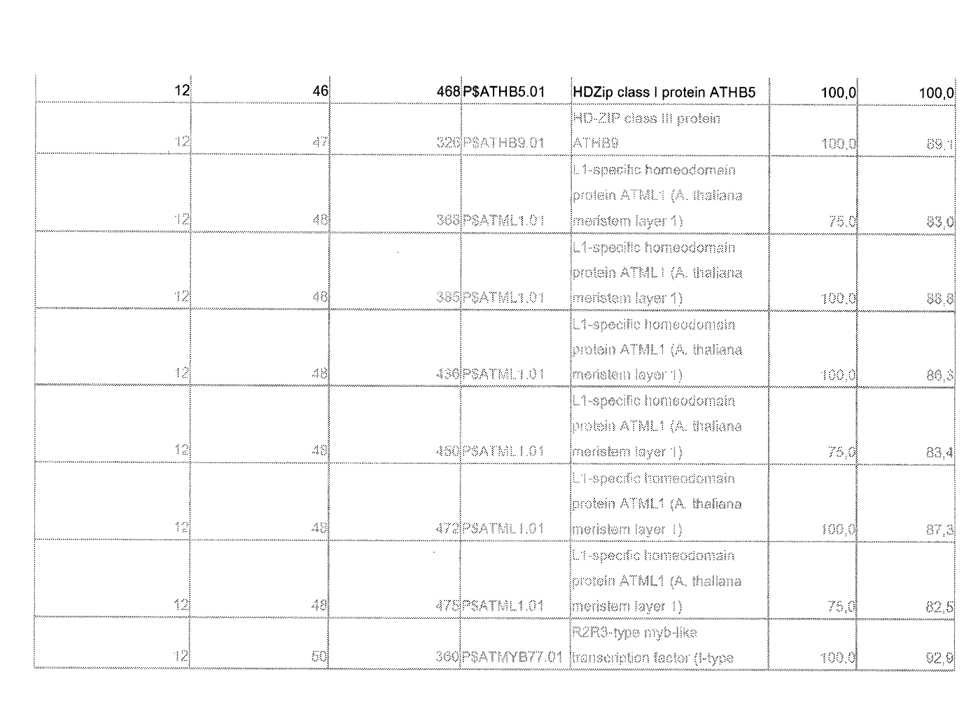

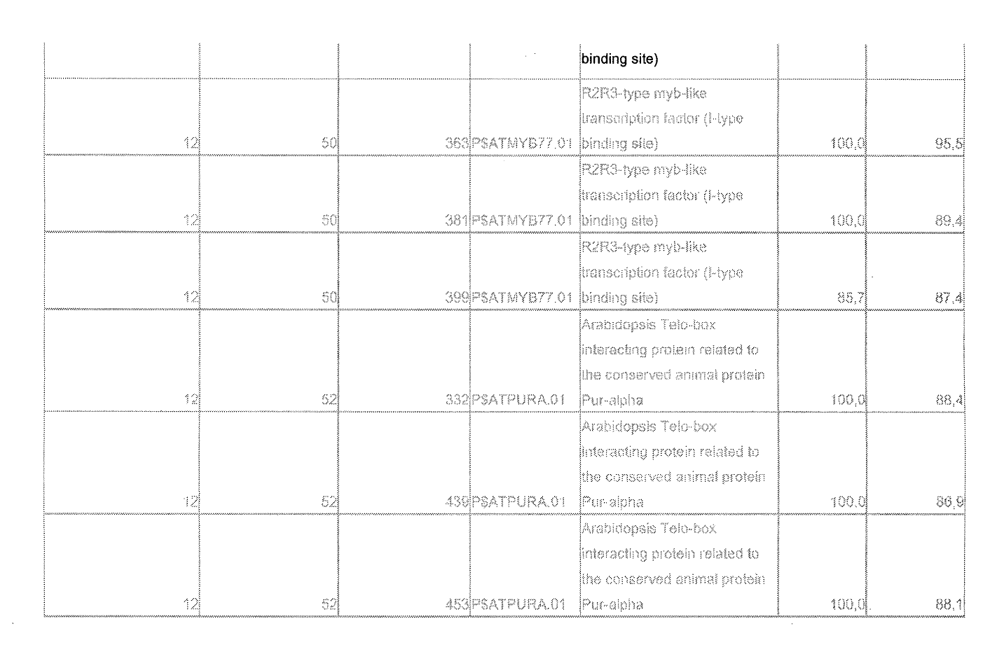

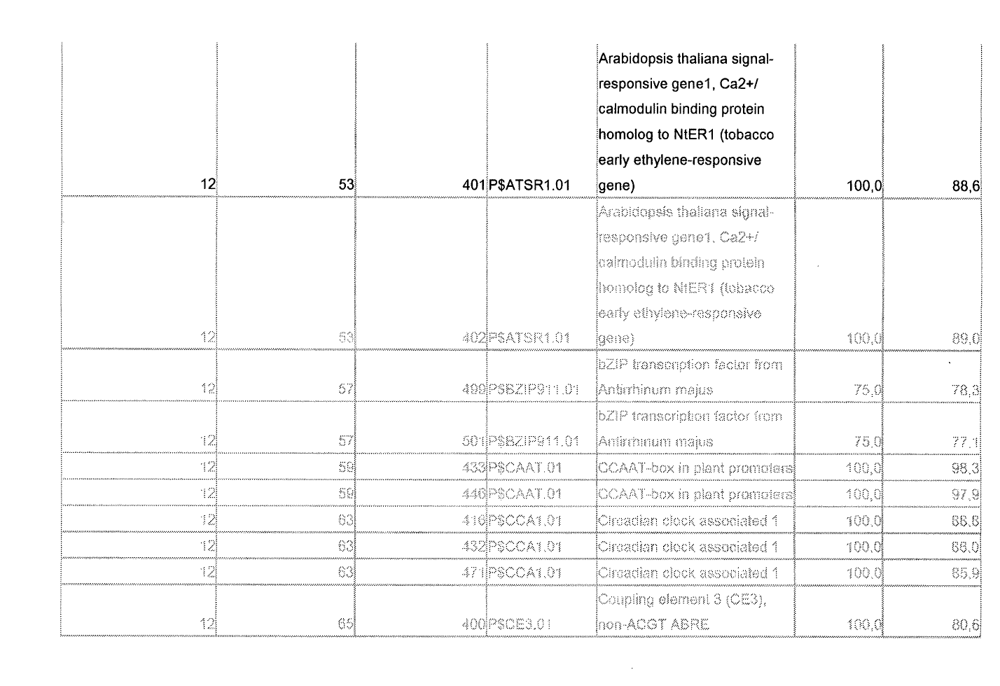

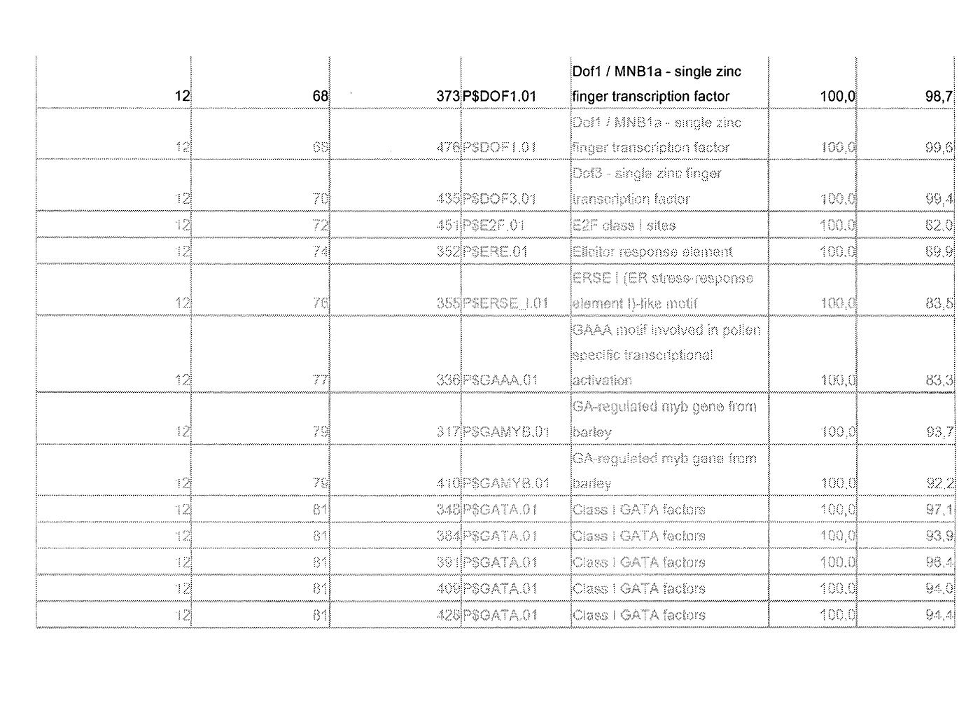

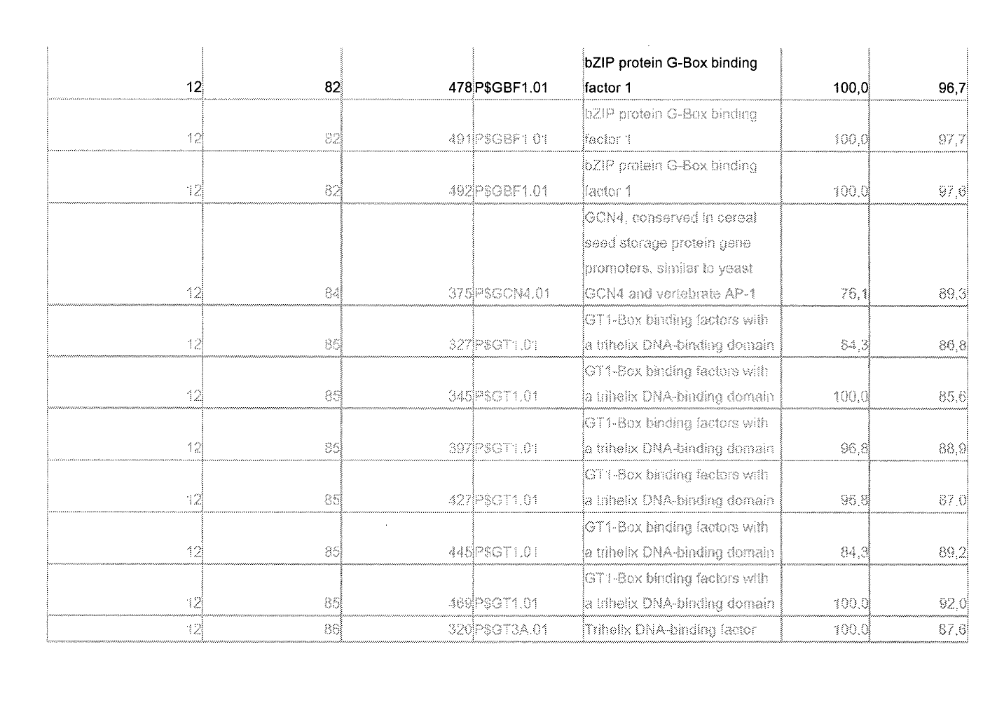

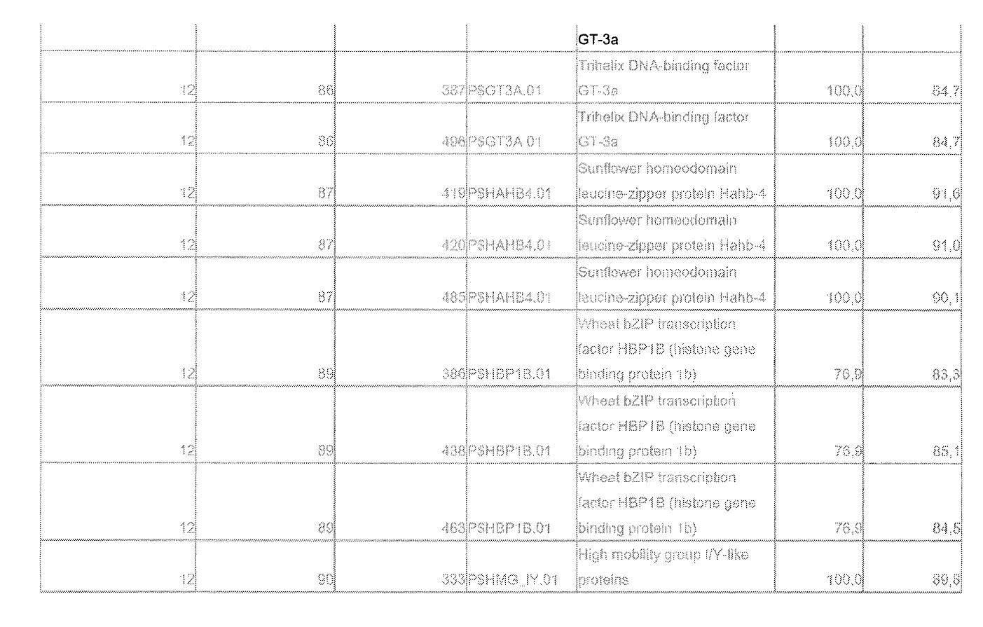

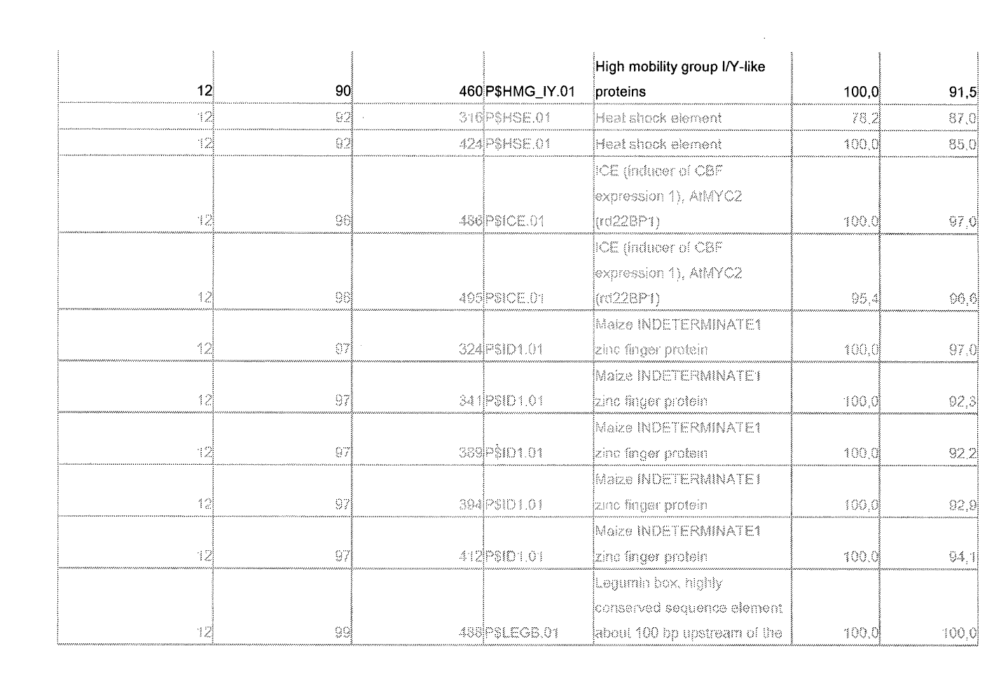

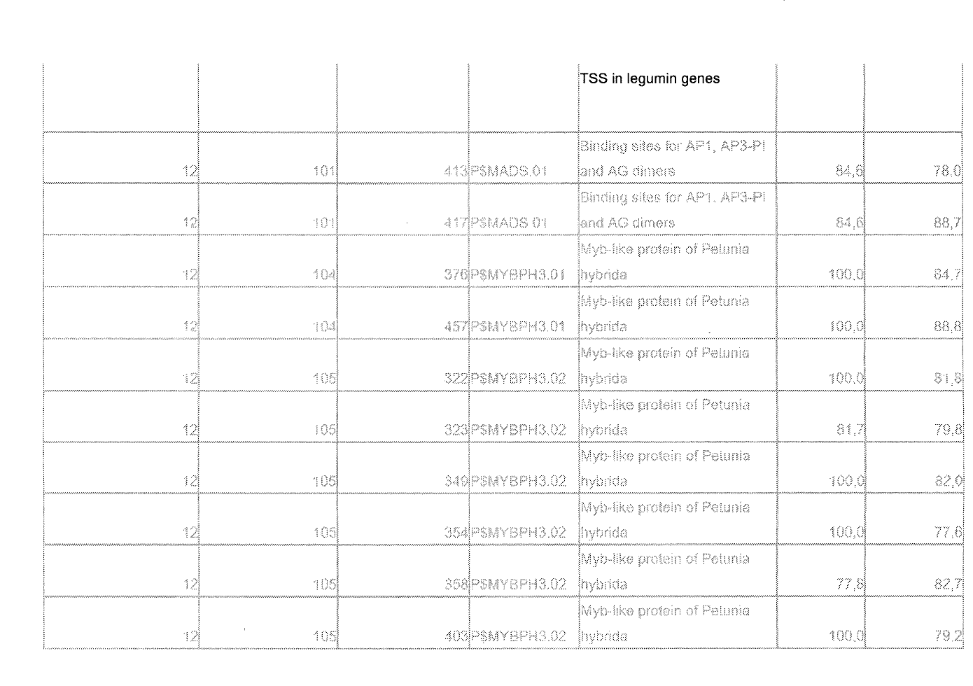

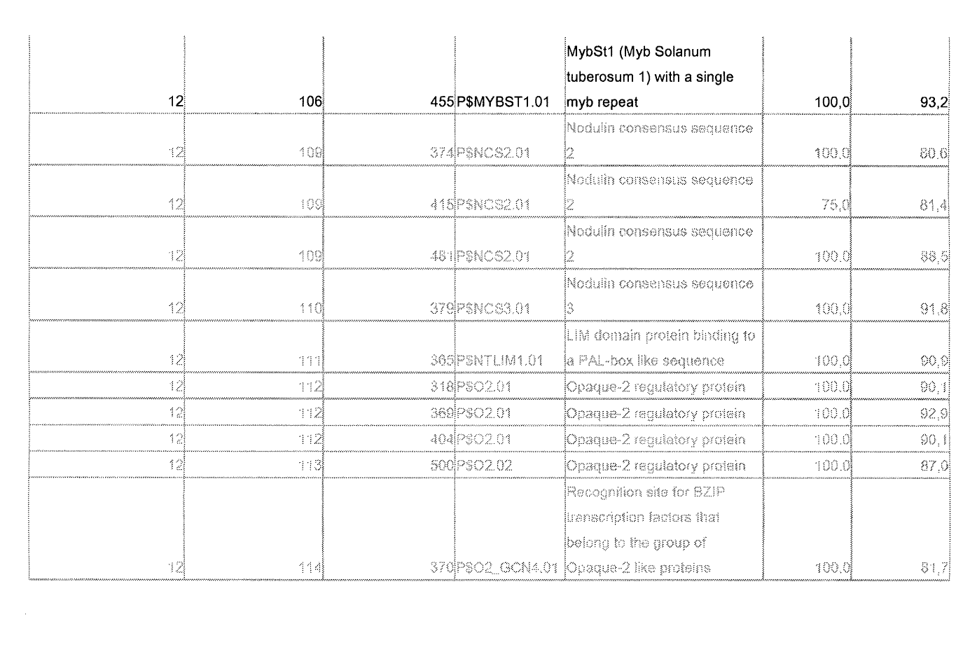

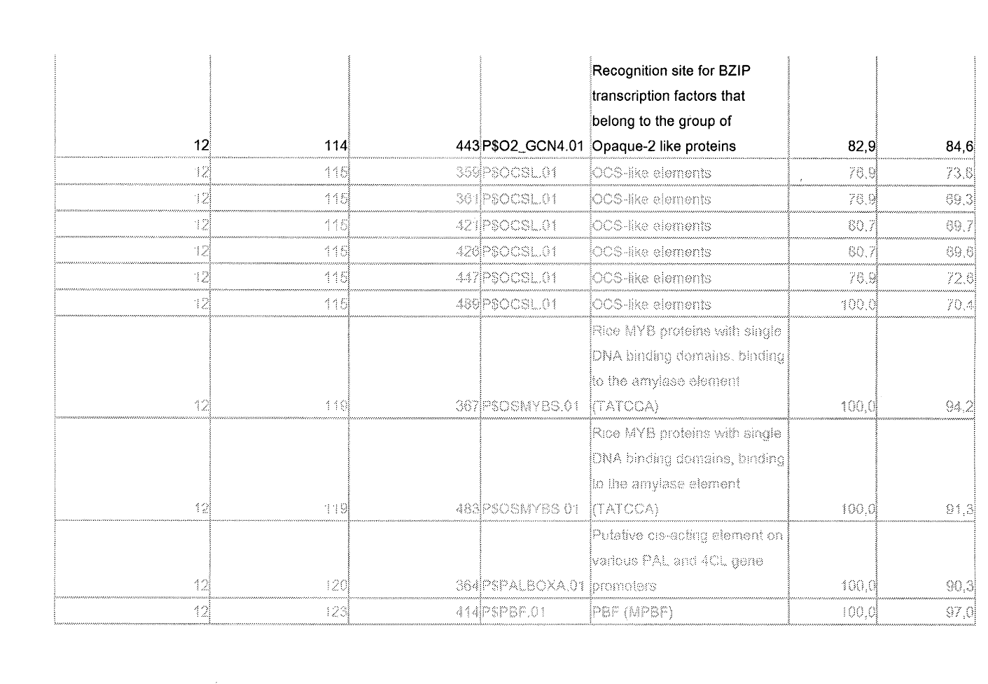

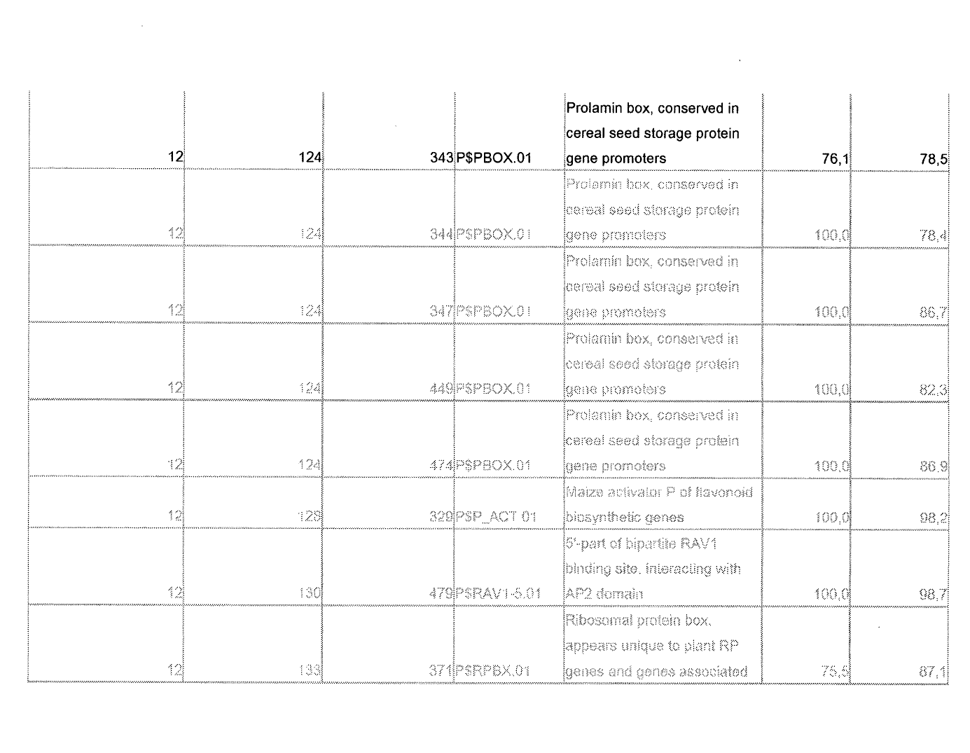

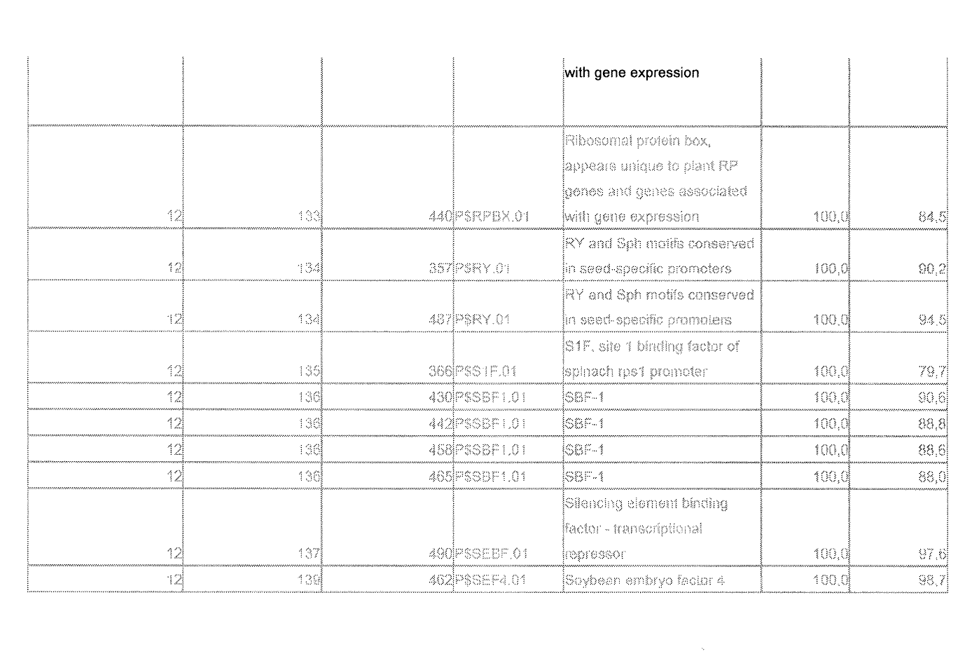

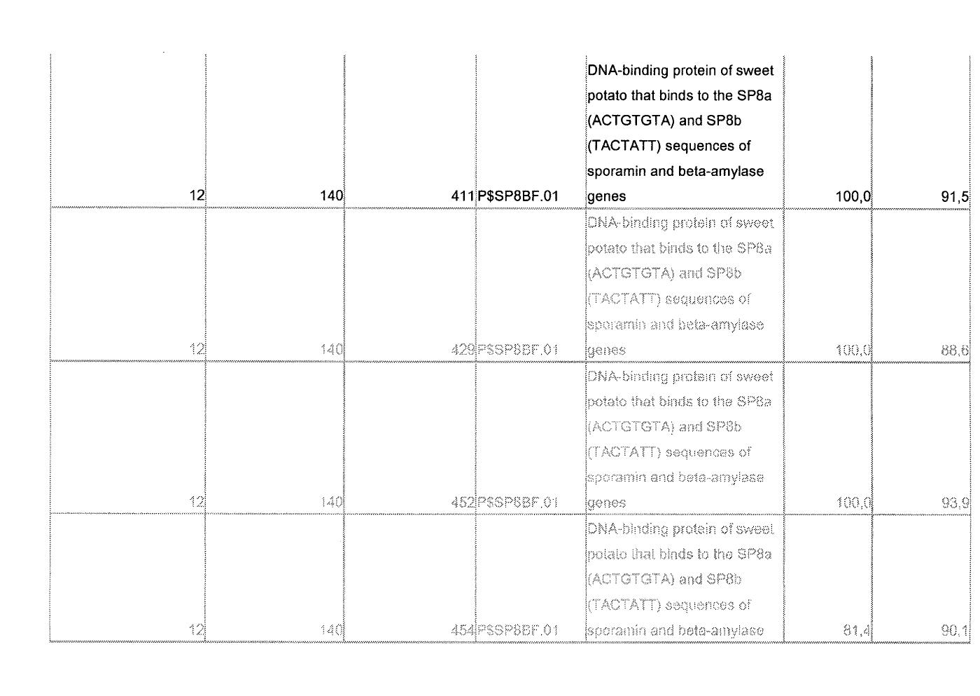

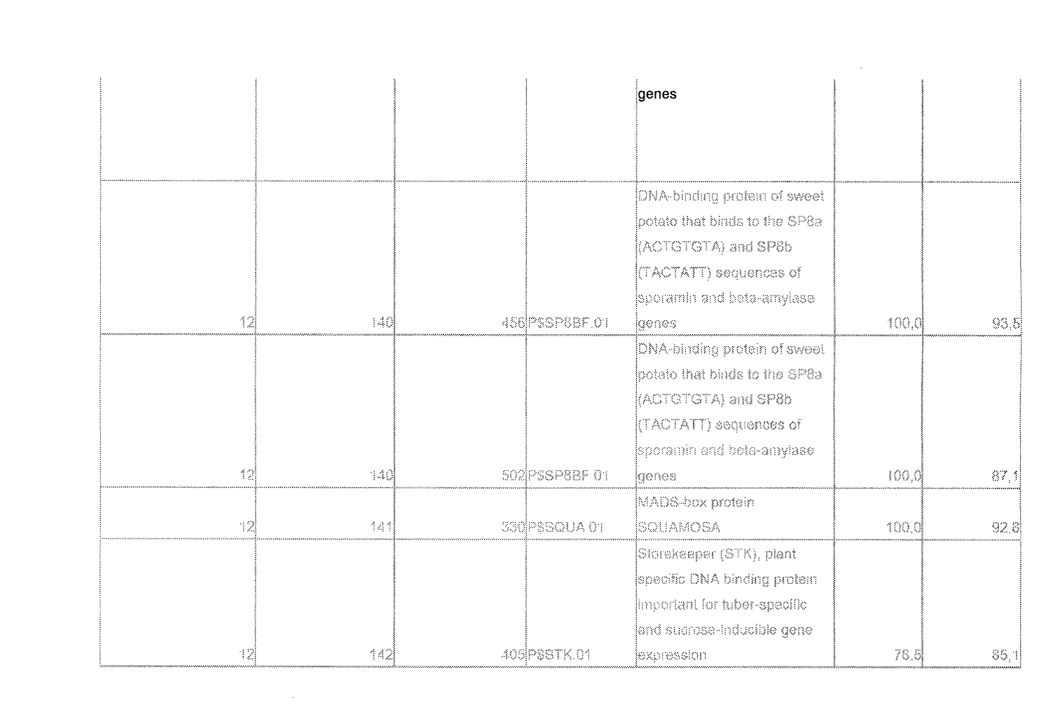

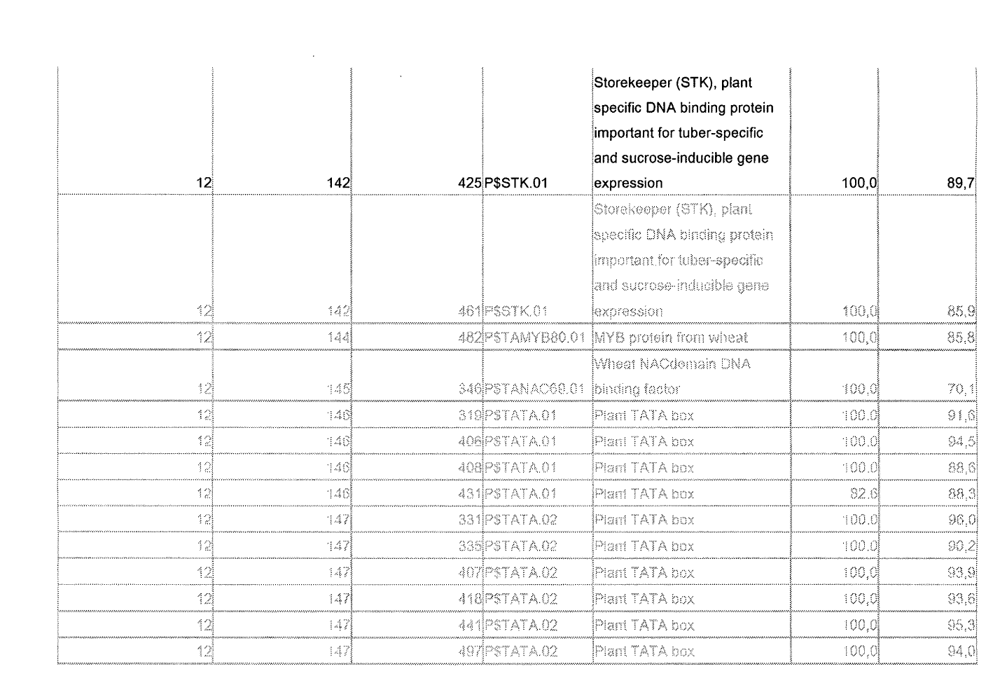

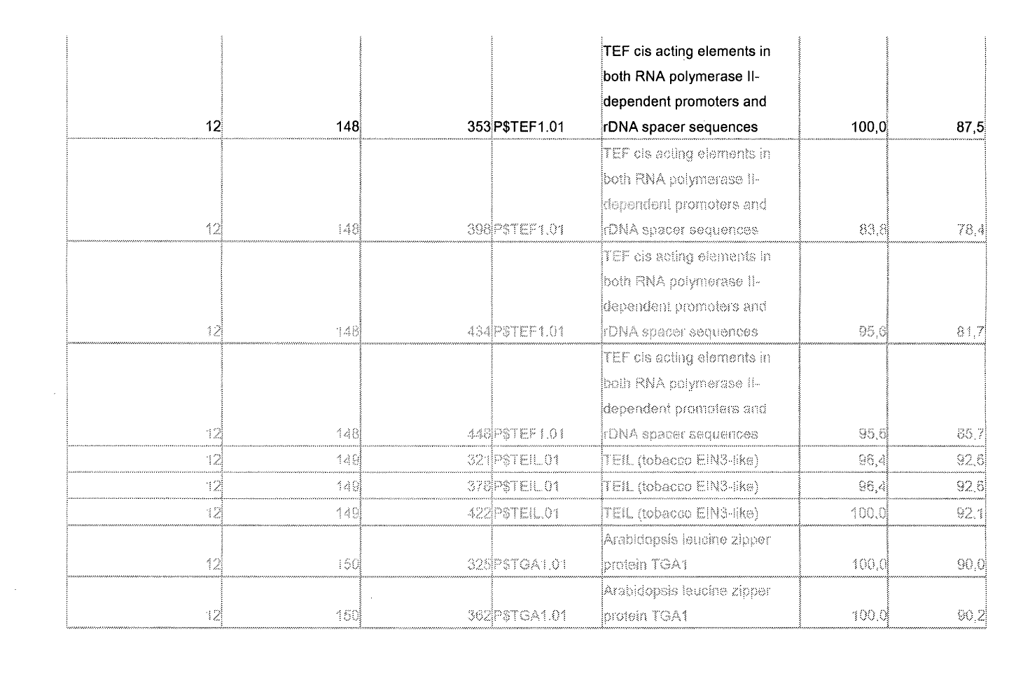

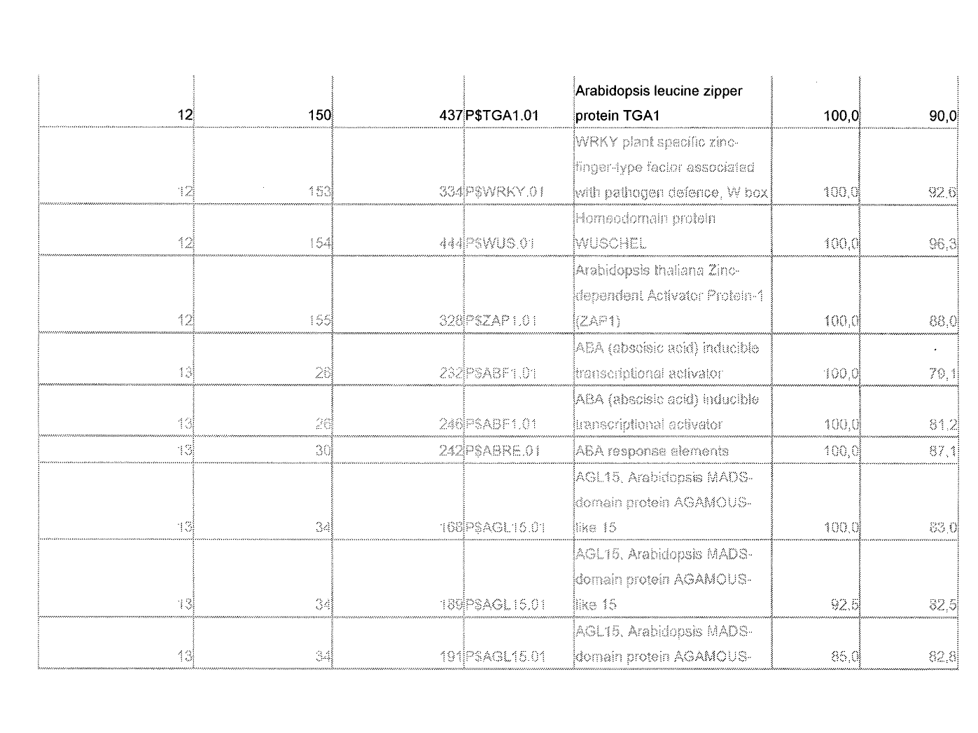

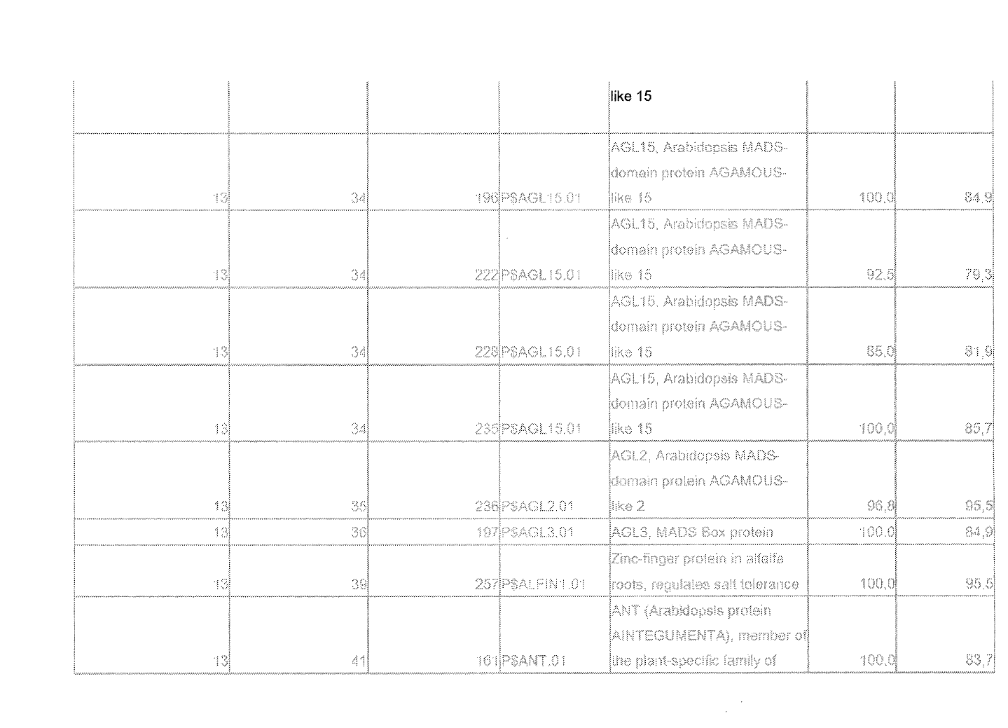

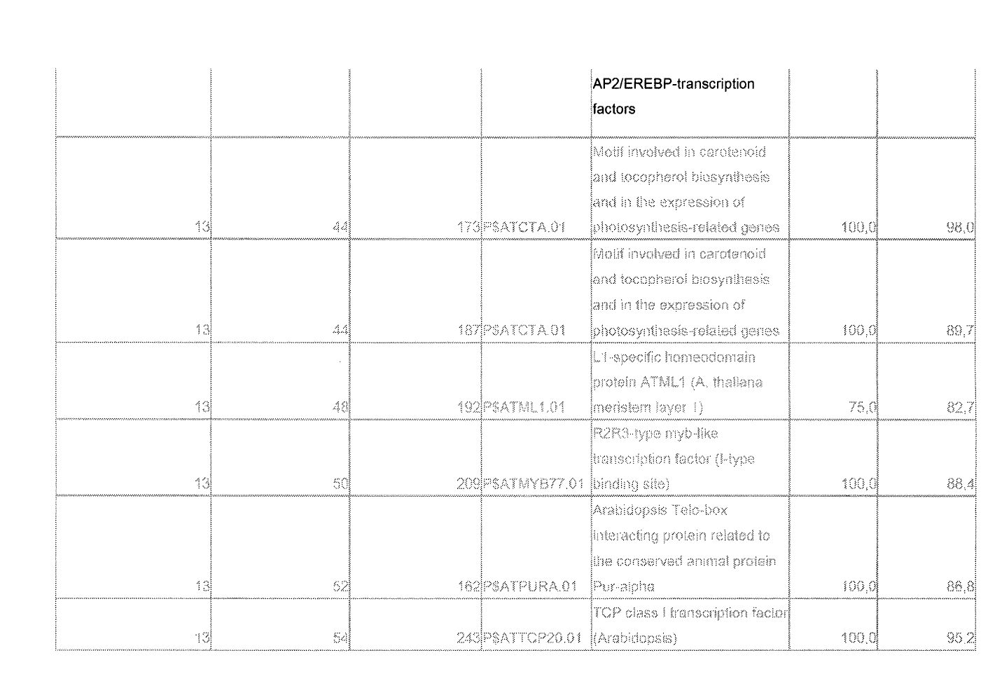

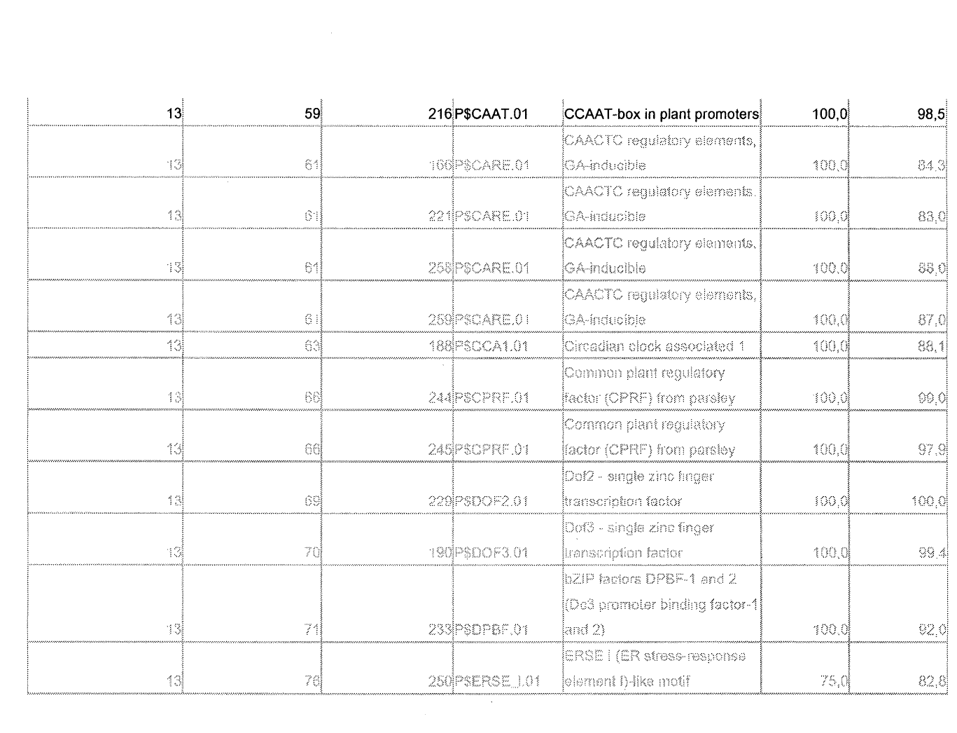

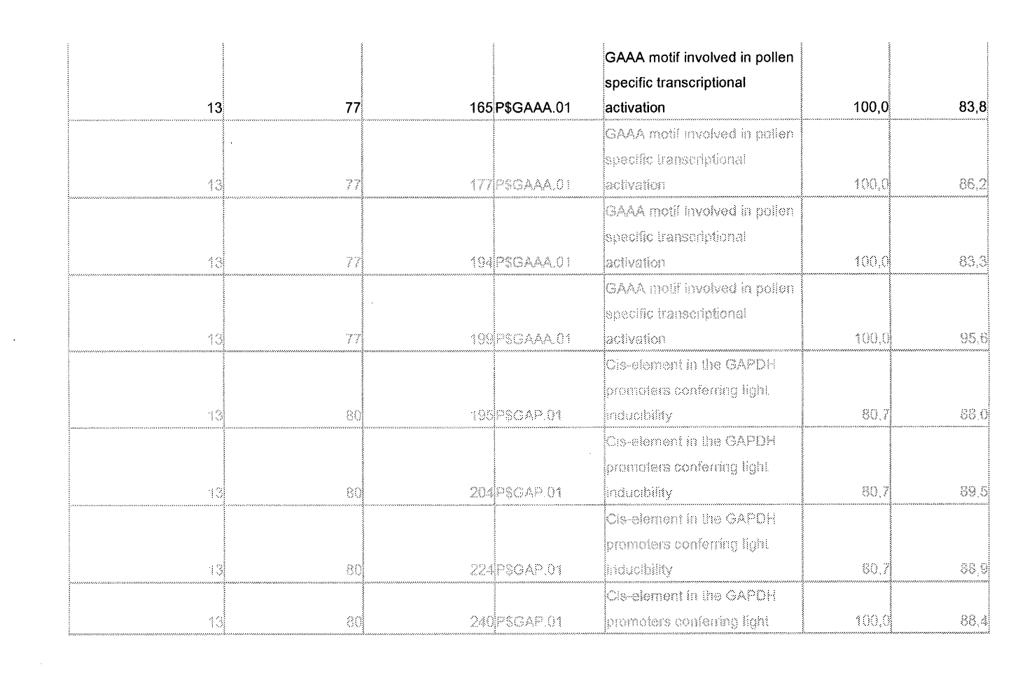

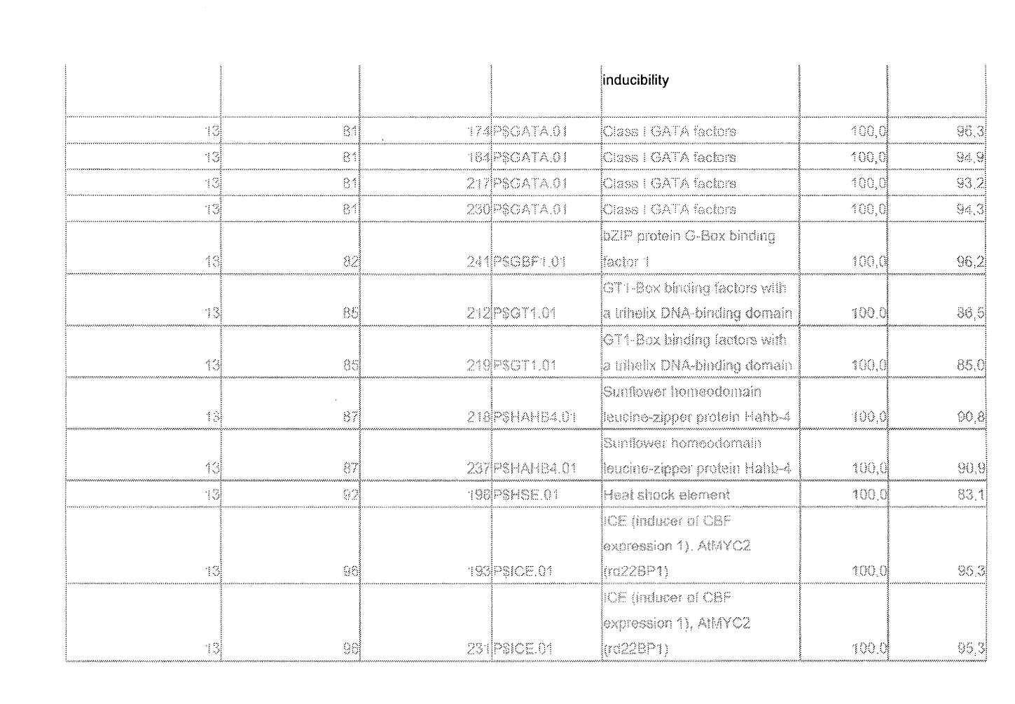

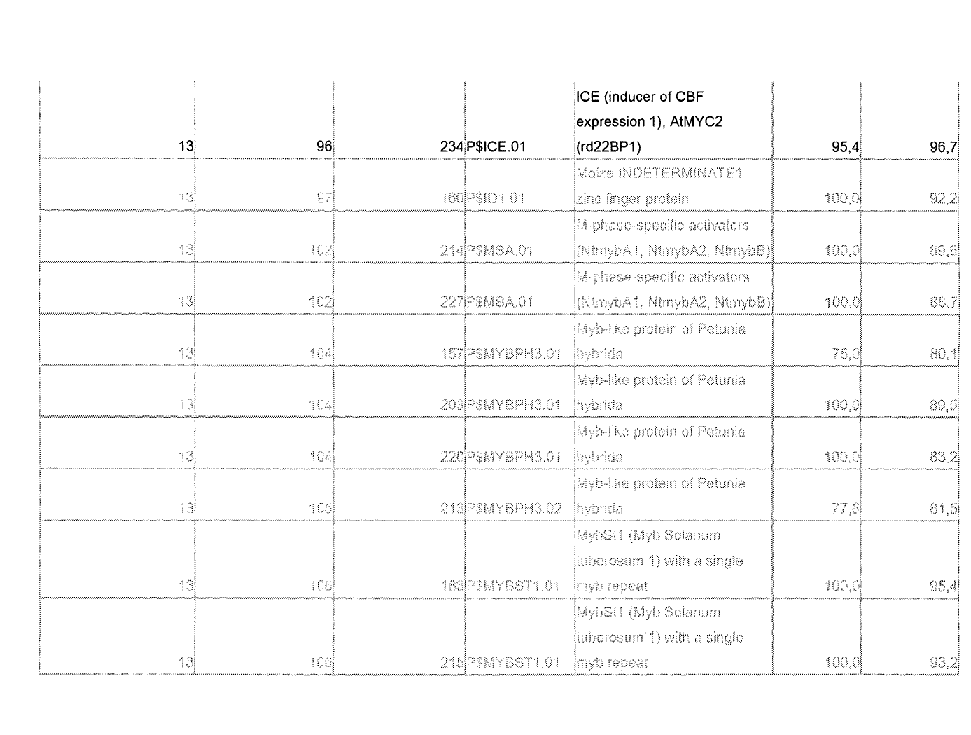

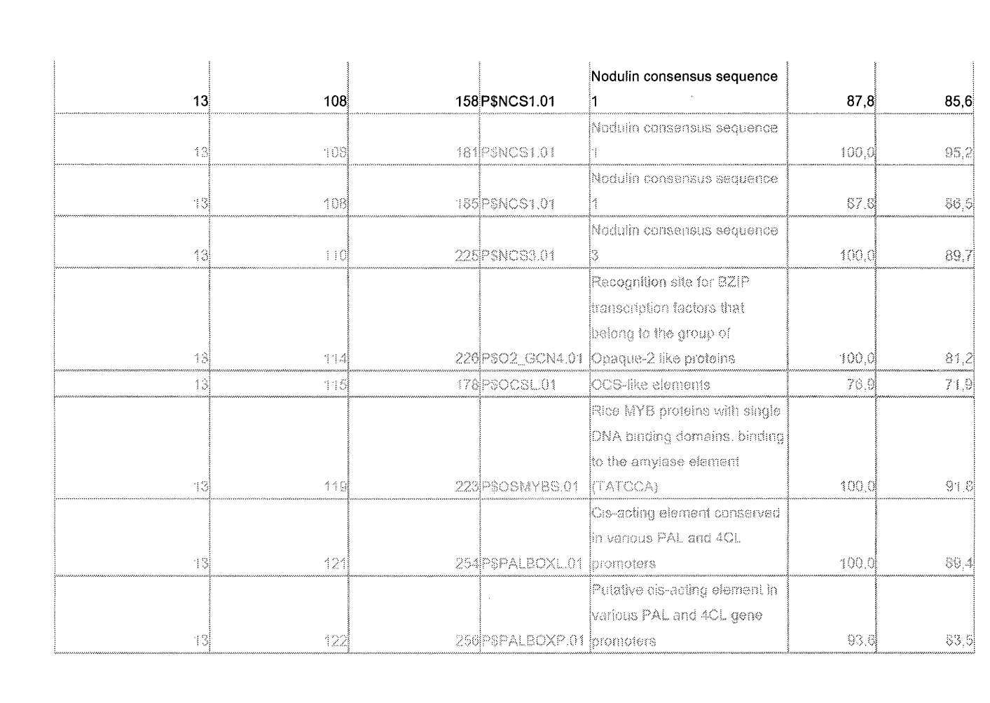

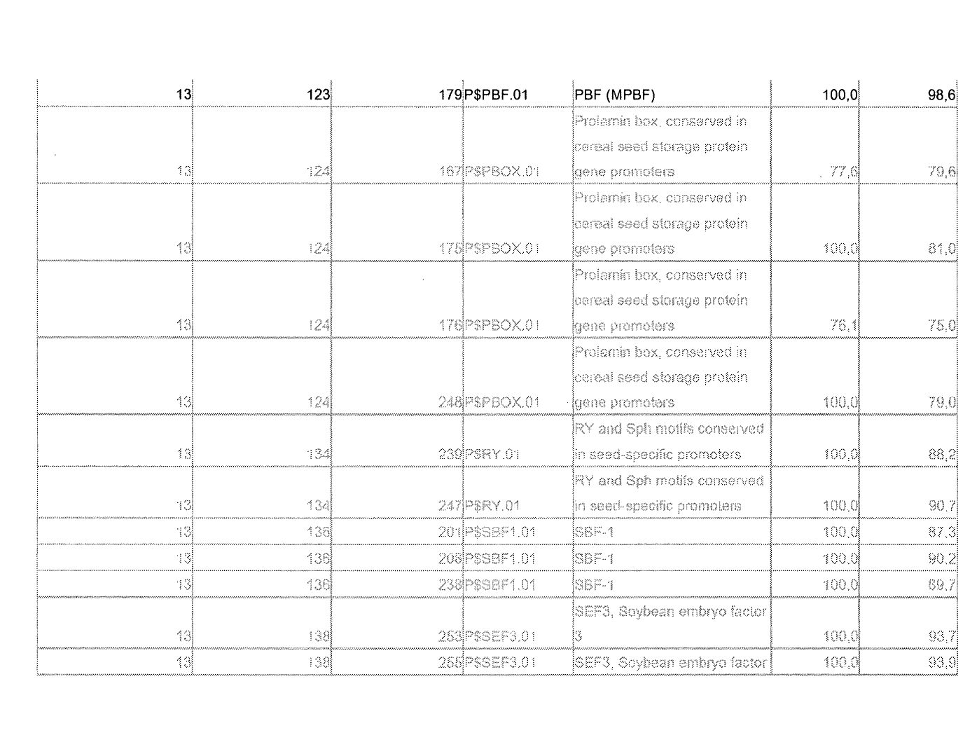

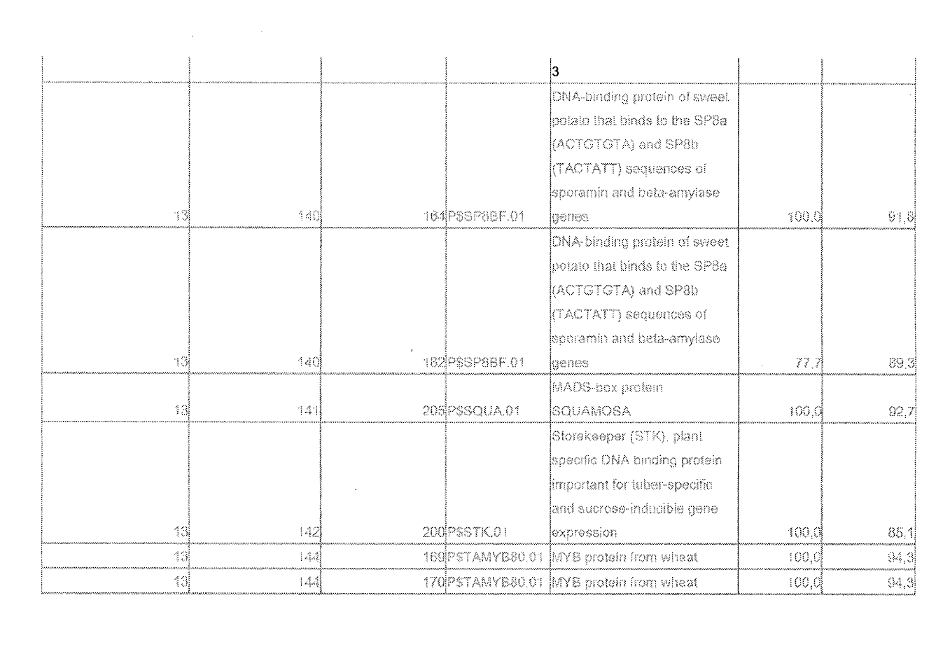

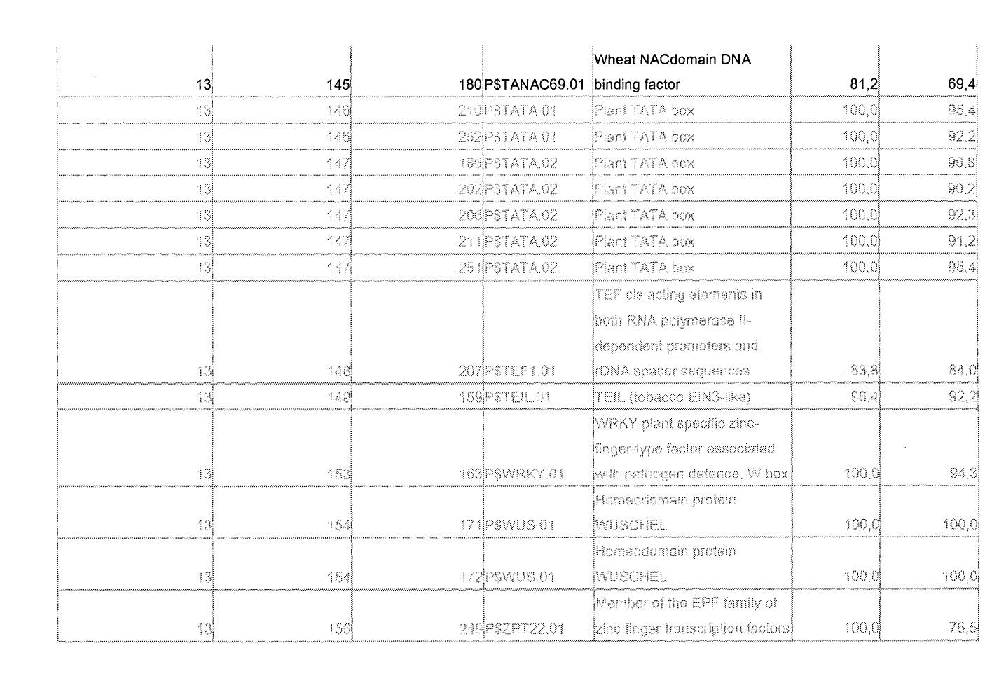

The results suggest that overexpression of the combination of LMPs as described in Table 9 of FIG. 8 allows the manipulation of total seed oil content. As an example, the results of the seed lipid analysis of combinations number 21, 23, 26, 27, 32 & 33 are shown in FIG. 6. As controls plants transformed with the empty vector, i.e. pSun2 without the combination of trait genes, were grown together with the plants harbouring the combinations of LMPs and their seeds analysed simultaneously.

As a further example the data shown in table 8 in FIG. 7 demonstrates that seed oil content of canola seed can significantly be increased by introduction of the combinations of LMPs as listed in table 9 of FIG. 8. T2 seeds of plants harbouring the combination of LMPs listed in table 8 were analysed for seed oil content by NIRS. Control plants were non-transgenic segregants grown together with the transgenic plants carrying the combination of LMPs. Only lines with an increase of more than 5% are shown. The p-values shown were calculated using simple t-test.

The effect of the genetic modification in plants on a desired seed storage compound (such as a sugar, lipid or fatty acid) can be assessed by growing the modified plant under suitable conditions and analyzing the seeds or any other plant organ for increased production of the desired product (i.e., a lipid or a fatty acid). Such analysis techniques are well known to one skilled in the art, and include spectroscopy, thin layer chromatography, staining methods of various kinds, enzymatic and microbiological methods, and analytical chromatography such as high performance liquid chromatography (see, for example, Ullman 1985, Encyclopedia of Industrial Chemistry, vol. A2, pp. 89-90 and 443-613, VCH: Weinheim; Fallon, A. et al. 1987, Applications of HPLC in Biochemistry in: Laboratory Techniques in Biochemistry and Molecular Biology, vol. 17; Rehm et al., 1993 Product recovery and purification, Biotechnology, vol. 3, Chapter III, pp. 469-714, VCH: Weinheim; Belter, P. A. et al., 1988 Bioseparations: downstream processing for biotechnology, John Wiley & Sons; Kennedy J. F. & Cabral J. M. S. 1992, Recovery processes for biological materials, John Wiley and Sons; Shaeiwitz J. A. & Henry J. D. 1988, Biochemical separations in: Ulmann's Encyclopedia of Industrial Chemistry, Separation and purification techniques in biotechnology, vol. B3, Chapter 11, pp. 1-27, VCH: Weinheim; and Dechow F. J. 1989).

Besides the above-mentioned methods, plant lipids are extracted from plant material as described by Cahoon et al. (1999, Proc. Natl. Acad. Sci. USA 96, 22:12935-12940) and Browse et al. (1986, Anal. Biochemistry 442:141-145). Qualitative and quantitative lipid or fatty acid analysis is described in Christie, William W., Advances in Lipid Methodology. Ayr/Scotland:Oily Press.—(Oily Press Lipid Library; Christie, William W., Gas Chromatography and Lipids. A Practical Guide—Ayr, Scotland:Oily Press, 1989 Repr. 1992.—IX, 307 S.—(Oily Press Lipid Library; and “Progress in Lipid Research,” Oxford:Pergamon Press, 1 (1952)-16 (1977) Progress in the Chemistry of Fats and Other Lipids CODEN.

Unequivocal proof of the presence of fatty acid products can be obtained by the analysis of transgenic plants following standard analytical procedures: GC, GC-MS or TLC as variously described by Christie and references therein (1997 in: Advances on Lipid Methodology 4th ed.: Christie, Oily Press, Dundee, pp. 119-169; 1998). Detailed methods are described for leaves by Lemieux et al. (1990, Theor. Appl. Genet. 80:234-240), and for seeds by Focks & Benning (1998, Plant Physiol. 118:91-101).

Positional analysis of the fatty acid composition at the sn-1, sn-2 or sn-3 positions of the glycerol backbone is determined by lipase digestion (see, e.g., Siebertz & Heinz 1977, Z. Naturforsch. 32c:193-205, and Christie 1987, Lipid Analysis 2nd Edition, Pergamon Press, Exeter, ISBN 0-08-023791-6).

Total seed oil levels can be measured by any appropriate method. Quantitation of seed oil contents is often performed with conventional methods, such as near infrared analysis (NIR) or nuclear magnetic resonance imaging (NMR). NIR spectroscopy has become a standard method for screening seed samples whenever the samples of interest have been amenable to this technique. Samples studied include canola, soybean, maize, wheat, rice, and others. NIR analysis of single seeds can be used (see e.g. Velasco et al., Estimation of seed weight, oil content and fatty acid composition in intact single seeds of rapeseed (Brassica napus L.) by near-infrared reflectance spectroscopy, Euphytica, Vol. 106, 1999, pp. 79-85). NMR has also been used to analyze oil content in seeds (see e.g. Robertson & Morrison, “Analysis of oil content of sunflower seed by wide-line NMR,” Journal of the American Oil Chemists Society, 1979, Vol. 56, 1979, pp. 961-964, which is herein incorporated by reference in its entirety).

A typical way to gather information regarding the influence of increased or decreased protein activities on lipid and sugar biosynthetic pathways is for example via analyzing the carbon fluxes by labeling studies with leaves or seeds using 14C-acetate or 14C-pyruvate (see, e.g. Focks & Benning 1998, Plant Physiol. 118:91-101; Eccleston & Ohlrogge 1998, Plant Cell 10:613-621). The distribution of carbon-14 into lipids and aqueous soluble components can be determined by liquid scintillation counting after the respective separation (for example on TLC plates) including standards like 14C-sucrose and 14C-malate (Eccleston & Ohlrogge 1998, Plant Cell 10:613-621).

Material to be analyzed can be disintegrated via sonification, glass milling, liquid nitrogen, and grinding, or via other applicable methods. The material has to be centrifuged after disintegration. The sediment is re-suspended in distilled water, heated for 10 minutes at 100° C., cooled on ice and centrifuged again followed by extraction in 0.5 M sulfuric acid in methanol containing 2% dimethoxypropane for 1 hour at 90° C. leading to hydrolyzed oil and lipid compounds resulting in transmethylated lipids. These fatty acid methyl esters are extracted in petrolether and finally subjected to GC analysis using a capillary column (Chrompack, WCOT Fused Silica, CP-Wax-52 CB, 25 m, 0.32 mm) at a temperature gradient between 170° C. and 240° C. for 20 minutes and 5 min. at 240° C. The identity of resulting fatty acid methylesters is defined by the use of standards available form commercial sources (i.e., Sigma).

In case of fatty acids where standards are not available, molecule identity is shown via derivatization and subsequent GC-MS analysis. For example, the localization of triple bond fatty acids is shown via GC-MS after derivatization via 4,4-Dimethoxy-oxazolin-Derivaten (Christie, Oily Press, Dundee, 1998).

A common standard method for analyzing sugars, especially starch, is published by Stitt M., Lilley R. Mc. C., Gerhardt R. and Heldt M. W. (1989, “Determination of metabolite levels in specific cells and subcellular compartments of plant leaves” Methods Enzymol. 174:518-552; for other methods see also Härtel et al. 1998, Plant Physiol. Biochem. 36:407-417 and Focks & Benning 1998, Plant Physiol. 118:91-101).

For the extraction of soluble sugars and starch, 50 seeds are homogenized in 500 μl of 80% (v/v) ethanol in a 1.5-ml polypropylene test tube and incubated at 70° C. for 90 min. Following centrifugation at 16,000 g for 5 min, the supernatant is transferred to a new test tube. The pellet is extracted twice with 500 μl of 80% ethanol. The solvent of the combined supernatants is evaporated at room temperature under a vacuum. The residue is dissolved in 50 μl of water, representing the soluble carbohydrate fraction. The pellet left from the ethanol extraction, which contains the insoluble carbohydrates including starch, is homogenized in 200 μl of 0.2 N KOH, and the suspension is incubated at 95° C. for 1 h to dissolve the starch. Following the addition of 35 μl of 1 N acetic acid and centrifugation for 5 min at 16,000, the supernatant is used for starch quantification.

To quantify soluble sugars, 10 μl of the sugar extract is added to 990 μl of reaction buffer containing 100 mM imidazole, pH 6.9, 5 mM MgCl2, 2 mM NADP, 1 mM ATP, and 2 units 2 ml-1 of Glucose-6-P-dehydrogenase. For enzymatic determination of glucose, fructose, and sucrose, 4.5 units of hexokinase, 1 unit of phosphoglucoisomerase, and 2 μl of a saturated fructosidase solution are added in succession. The production of NADPH is photometrically monitored at a wavelength of 340 nm. Similarly, starch is assayed in 30 μl of the insoluble carbohydrate fraction with a kit from Boehringer Mannheim.

An example for analyzing the protein content in leaves and seeds can be found by Bradford M. M. (1976, “A rapid and sensitive method for the quantification of microgram quantities of protein using the principle of protein dye binding,” Anal. Biochem. 72:248-254). For quantification of total seed protein, 15-20 seeds are homogenized in 250 μl of acetone in a 1.5-ml polypropylene test tube. Following centrifugation at 16,000 g, the supernatant is discarded and the vacuum-dried pellet is resuspended in 250 μl of extraction buffer containing 50 mM Tris-HCl, pH 8.0, 250 mM NaCl, 1 mM EDTA, and 1% (w/v) SDS. Following incubation for 2 h at 25° C., the homogenate is centrifuged at 16,000 g for 5 min and 200 ml of the supernatant will be used for protein measurements. In the assay, γ-globulin is used for calibration. For protein measurements, Lowry DC protein assay (Bio-Rad) or Bradford-assay (Bio-Rad) is used.

Enzymatic assays of hexokinase and fructokinase are performed spectropho-tometrically according to Renz et al. (1993, Planta 190:156-165), of phosphogluco-isomerase, ATP-dependent 6-phosphofructokinase, pyrophosphate-dependent 6-phospho-fructokinase, Fructose-1,6-bisphosphate aldolase, triose phosphate isomerase, glyceral-3-P dehydrogenase, phosphoglycerate kinase, phosphoglycerate mutase, enolase, and pyruvate kinase are performed according to Burrell et al. (1994, Planta 194:95-101) and of UDP-Glucose-pyrophosphorylase according to Zrenner et al. (1995, Plant J. 7:97-107).

Intermediates of the carbohydrate metabolism, like Glucose-1-phosphate, Glucose-6-phosphate, Fructose-6-phosphate, Phosphoenolpyruvate, Pyruvate, and ATP are measured as described in Härtel et al. (1998, Plant Physiol. Biochem. 36:407-417) and metabolites are measured as described in Jelitto et al. (1992, Planta 188:238-244).

In addition to the measurement of the final seed storage compound (i.e., lipid, starch or storage protein) it is also possible to analyze other components of the metabolic pathways utilized for the production of a desired seed storage compound, such as intermediates and side-products, to determine the overall efficiency of production of the compound (Fiehn et al. 2000, Nature Biotech. 18:1447-1161).

For example, yeast expression vectors comprising the nucleic acids disclosed herein, or fragments thereof, can be constructed and transformed into using standard protocols. The resulting transgenic cells can then be assayed for alterations in sugar, oil, lipid, or fatty acid contents.

Similarly, plant expression vectors comprising the nucleic acids disclosed herein, or fragments thereof, can be constructed and transformed into an appropriate plant cell such as Arabidopsis, soybean, rapeseed, rice, maize, wheat, Medicago truncatula, etc., using standard protocols. The resulting transgenic cells and/or plants derived there from can then be assayed for alterations in sugar, oil, lipid or fatty acid contents.

Additionally, the combinations of sequences disclosed herein, or fragments thereof, can be used to generate knockout mutations in the genomes of various organisms, such as bacteria, mammalian cells, yeast cells, and plant cells (Girke at al. 1998, Plant J. 15:39-48). The resultant knockout cells can then be evaluated for their composition and content in seed storage compounds, and the effect on the phenotype and/or genotype of the mutation. For other methods of gene inactivation include U.S. Pat. No. 6,004,804 “Non-Chimeric Mutational Vectors” and Puttaraju et al. (1999, “Spliceosome-mediated RNA trans-splicing as a tool for gene therapy,” Nature Biotech. 17:246-252).

### Example 10

Purification of the Desired Products from Transformed Organisms. LMPs can be recovered from plant material by various methods well known in the art. Organs of plants can be separated mechanically from other tissue or organs prior to isolation of the seed storage compound from the plant organ. Following homogenization of the tissue, cellular debris is removed by centrifugation and the supernatant fraction containing the soluble proteins is retained for further purification of the desired compound. If the product is secreted from cells grown in culture, then the cells are removed from the culture by low-speed centrifugation and the supernate fraction is retained for further purification.

The supernatant fraction from either purification method is subjected to chromatography with a suitable resin, in which the desired molecule is either retained on a chromatography resin, while many of the impurities in the sample are not, or where the impurities are retained by the resin, while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There is a wide array of purification methods known to the art and the preceding method of purification is not meant to be limiting. Such purification techniques are described, for example, in Bailey J. E. & Ollis D. F. 1986, Biochemical Engineering Fundamentals, McGraw-Hill:New York).

The identity and purity of the isolated compounds may be assessed by techniques standard in the art. These include high-performance liquid chromatography (HPLC), spectroscopic methods, staining methods, thin layer chromatography, analytical chromatography such as high performance liquid chromatography, NIRS, enzymatic assay, or microbiologically. Such analysis methods are reviewed in: Patek et al. (1994, Appl. Environ. Microbiol. 60:133-140), Malakhova et al. (1996, Biotekhnologiya 11:27-32) and Schmidt et al. (1998, Bioprocess Engineer 19:67-70), Ulmann's Encyclopedia of Industrial Chemistry (1996, Vol. A27, VCH: Weinheim, p. 89-90, p. 521-540, p. 540-547, p. 559-566, 575-581 and p. 581-587) and Michal G. (1999, Biochemical Pathways: An Atlas of Biochemistry and Molecular Biology, John Wiley and Sons; Fallon, A. et al. 1987, Applications of HPLC in Biochemistry in: Laboratory Techniques in Biochemistry and Molecular Biology, vol. 17).

Those skilled in the art will recognize, or will be able to ascertain using no more than routine experimentation, many equivalents to the specific embodiments of the invention described herein. Such equivalents are intended to be encompassed by the claims to the invention disclosed and claimed herein.

TABLE 2
Plant Lipid Classes

| | |
|---|---|
| Neutral Lipids | Triacylglycerol (TAG) |
| | Diacylglycerol (DAG) |
| | Monoacylglycerol (MAG) |
| Polar Lipids | Monogalactosyldiacylglycerol (MGDG) |
| | Digalactosyldiacylglycerol (DGDG) |
| | Phosphatidylglycerol (PG) |
| | Phosphatidylcholine (PC) |
| | Phosphatidylethanolamine (PE) |
| | Phosphatidylinositol (PI) |
| | Phosphatidylserine (PS) |
| | Sulfoquinovosyldiacylglycerol |

TABLE 3
Common Plant Fatty Acids

| | |
|---|---|
| 16:0 | Palmitic acid |
| 16:1 | Palmitoleic acid |
| 16:3 | Palmitolenic acid |
| 18:0 | Stearic acid |
| 18:1 | Oleic acid |
| 18:2 | Linoleic acid |
| 18:3 | Linolenic acid |
| γ-18:3 | Gamma-linolenic acid * |
| 20:0 | Arachidic acid |
| 20:1 | Eicosenoic acid |
| 22:6 | Docosahexanoic acid (DHA) * |
| 20:2 | Eicosadienoic acid |
| 20:4 | Arachidonic acid (AA) * |
| 20:5 | Eicosapentaenoic acid (EPA) * |
| 22:1 | Erucic acid |

* These fatty acids do not normally occur in plant seed oils, but their production in transgenic plant seed oil is of importance in plant biotechnology.

TABLE 4
A table of the putative functions of the LMPs (the full length nucleic acid
sequences can be found in Appendix A using the sequence codes, column 2 shows
the concordance of the sequence identifier used in Appendix A with or the
sequence identifier of the WIPO Standard ST. 25 sequence listing)

| | SEQ ID as used in | | | |
|---|---|---|---|---|
| Seq ID as | WIPO Standard | | | |
| used in | ST. 25 sequence | | | |
| Appendix A | listing | Sequence name | Species | Function |
| 1 | 1 | Wri | Arabidopsis | wrinkle transcription factor |
| | | | thaliana | |
| 2 | 3 | JB05 | Arabidopsis | beta-ketoacyl-CoA synthase |
| | | | thaliana | |
| 3 | 5 | JB4054 | Arabidopsis | enoyl CoA |
| | | | thaliana | hydratase/isomerase |
| 4 | 7 | CTR1 | Arabidopsis | Regulator of ethylene |
| | | | thaliana | response |
| 5 | 9 | CK | Physcomitrella | Protein kinase |
| | | | patens | |
| 3 | 11 | DGD | Arabidopsis | Phospholipid metabolism |
| | | | thaliana | |
| 7 | 13 | Susy | Arabidopsis | Sucrose synthase |
| | | | thaliana | |
| 8 | 15 | PCT | Arabidopsis | Phospholipid metabolism |
| | | | thaliana | |

Table 5 with concordance of sequence identifiers used for promoters of appendix A

| | SEQ ID as used in WIPO | |
|---|---|---|
| Seq ID as used | Standard ST. 25 sequence | |
| in Appendix A | listing | Sequence name |
| 9 | 17 | PtxA |
| 10 | 18 | USP |
| 11 | 19 | LeB4 |
| 12 | 20 | LEB4 |
| 13 | 21 | Conlinin |

Table 6 with concordance of sequence identifiers used for terminators of Appendix A

| | SEQ ID as used in WIPO | |
|---|---|---|
| Seq ID as used | Standard ST. 25 sequence | |
| in Appendix A | listing | Sequence name |
| 14 | 22 | E9 |
| 15 | 23 | A7 |
| 16 | 24 | OCS |
| 17 | 25 | LeBT |

TABLE 7
Maximum oil increase observed in T2 Arabidopsis seed of
transgenic plants carrying the combinations of LMPs

| | Maximal relative oil increase observed in |
|---|---|
| Combination of LMPs | a line as % of the control value |
| 23 | 112.6 |
| 26 | 120.1 |
| 27 | 116.3 |
| 32 | 109.1 |
| 33 | 111.3 |

1. An isolated nucleic acid comprising two or more LMP polynucleotide sequences selected from the group consisting of:
a. a polynucleotide sequence as described by SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8;
b. a polynucleotide sequence encoding a polypeptide as described by SEQ ID NO: 18, SEQ ID NO: 19, SEQ ID NO: 20 or SEQ ID NO: 21, SEQ ID NO: 22, SEQ ID NO: 23, SEQ ID NO: 24 or SEQ ID NO: 25;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

2. An isolated polypeptide encoded by a polynucleotide sequence as claimed in claim 1.

3. The isolated nucleic acid of claim 1, wherein the isolated nucleic acid encodes a polypeptide that functions as a modulator of a seed storage compound in microorganisms or plants.

4. The isolated polypeptide of claim 2, wherein the isolated polypeptide sequence functions as a modulator of a seed storage compound in microorganisms or plants.

5. An expression vector containing the nucleic acid of claim 1, wherein the nucleic acid is operatively linked to a promoter selected from the group consisting of a seed-specific promoter, a root-specific promoter, and a non-tissue-specific promoter.

6. The expression vector of claim 5, wherein the promoter is selected from the group consisting of:
a. a polynucleotide sequence as described by SEQ ID NO: 9, SEQ ID NO: 10, SEQ ID NO: 11, SEQ ID NO: 12 or SEQ ID NO: 13;
b. a polynucleotide sequence having at least 70% sequence identity with the nucleic acid of a) above;
c. a polynucleotide sequence that hybridizes under stringent conditions to nucleic acid of a) or b) above;
d. a polynucleotide sequence comprising at least 50% by number of the polynucleotide sequences of the full-length polynucleotide sequence as described by SEQ ID NO: 26-156 related to a promoter as described by columns 1 and 2 of table 10; and
e. a polynucleotide sequence comprising a polynucleotide sequence having at least 70% sequence identity with the full length polynucleotide sequence as described by the capital letters of the polynucleotide sequence as described by SEQ ID NO: 26-156, wherein the polynucleotide sequence comprises 50% of the nucleotide sequences having at least 70% sequence identity with the polynucleotide sequence as described by the capital letters of the polynucleotide sequence as described by SEQ ID NO: 26-156 related to a promoter as described by columns 1 and 2 of table 10.

7. The expression vector of claim 5, wherein the terminator is selected from the group consisting of:
a. a polynucleotide sequence as described by SEQ ID NO: 14, SEQ ID NO: 15, SEQ ID NO: 16 or SEQ ID NO: 17;
b. a polynucleotide sequence having at least 70% sequence identity with the nucleic acid of a) above;
c. a polynucleotide sequence that is complementary to the nucleic acid of a) or b) above; and
d. a polynucleotide sequence that hybridizes under stringent conditions to the nucleic acid of a) or b) above.

8. A method of producing a transgenic plant having a modified level of a seed storage compound weight percentage compared to an empty vector control comprising,
a. a first step of introducing into a plant cell an expression vector containing a nucleic acid, and
b. a further step of generating from the plant cell the transgenic plant,
wherein the nucleic acid encodes a polypeptide that functions as a modulator of a seed storage compound in the plant, and wherein the nucleic acid comprises the polynucleotide sequence of claim 1.

9. The method of claim 8, wherein the nucleic acid comprises a polynucleotide sequence having at least 90% sequence identity with
a. a polynucleotide sequence as described by SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8; or
b. a polynucleotide sequence encoding a polypeptide as described by SEQ ID NO: 18, SEQ ID NO: 19, SEQ ID NO: 20 or SEQ ID NO: 21, SEQ ID NO: 22, SEQ ID NO: 23, SEQ ID NO: 24 or SEQ ID NO: 25.

10. The method of claim 8, wherein the total seed oil content weight percentage is increased in the transgenic plant as compared to an empty vector control.

11. A method of modulating the level of a seed storage compound weight percentage in a plant comprising, modifying the expression of a nucleic acid in the plant, comprising
a. a first step of introducing into a plant cell an expression vector comprising a nucleic acid, and
b. a further step of generating from the plant cell the transgenic plant,
wherein the nucleic acid encodes a polypeptide that functions as a modulator of a seed storage compound in the plant wherein the nucleic acid comprises the polynucleotide sequence of claim 1.

12. The method of claim 11, wherein the total seed oil content weight percentage is increased in the transgenic plant as compared to an empty vector control.

13. A transgenic plant made by the method of claim 8.

14. The transgenic plant of claim 13, wherein the total seed oil content weight percentage is increased in the transgenic plant as compared to an empty vector control.

15. The transgenic plant of claim 13, wherein the plant is selected from the group consisting of rapeseed, canola, linseed, soybean, sunflower, maize, oat, rye, barley, wheat, rice, pepper, tagetes, cotton, oil palm, coconut palm, flax, castor, sugarbeet, rice and peanut.

16. A seed produced by the transgenic plant of claim 13, wherein the plant expresses the polypeptide that functions as a modulator of a seed storage compound and wherein the plant is true breeding for a modified level of seed storage compound weight percentage as compared to an empty vector control.

17. A transgenic plant made by the method of claim 11.

18. The transgenic plant of claim 17, wherein the total seed oil content weight percentage is increased in the transgenic plant as compared to an empty vector control.

19. The transgenic plant of claim 17, wherein the plant is selected from the group consisting of rapeseed, canola, linseed, soybean, sunflower, maize, oat, rye, barley, wheat, rice, pepper, tagetes, cotton, oil palm, coconut palm, flax, castor, sugarbeet, rice and peanut.

20. A seed produced by the transgenic plant of claim 17, wherein the plant expresses the polypeptide that functions as a modulator of a seed storage compound and wherein the plant is true breeding for a modified level of seed storage compound weight percentage as compared to an empty vector control.
